(19) United States
(12) Patent Application Publication
(10) Pub. No.: US 2013/0029983 A1
(43) Pub. Date: Jan. 31, 2013

(54) AMINOTHIENOPYRIDAZINE INHIBITORS OF TAU ASSEMBLY

(71) Applicants: Carlo Ballatore, Philadelphia, PA (US); Kurt R. Brunden, Media, PA (US); Alexander L. Crowe, Media, PA (US); Donna M. Huryn, Upper Freehold, NJ (US); Virginia M.Y. Lee, Philadelphia, PA (US); John Q. Trojanowski, Philadelphia, PA (US); Amos B. Smith, III, Merion, PA (US); Ruili Huang, Rockville, MD (US); Wenwei Huang, Rockville, MD (US); Ronald L. Johnson, Derwood, MD (US); Francesco Piscitelli, Marina Di Stongoli (IT)

(72) Inventors: Carlo Ballatore, Philadelphia, PA (US); Kurt R. Brunden, Media, PA (US); Alexander L. Crowe, Media, PA (US); Donna M. Huryn, Upper Freehold, NJ (US); Virginia M.Y. Lee, Philadelphia, PA (US); John Q. Trojanowski, Philadelphia, PA (US); Amos B. Smith, III, Merion, PA (US); Ruili Huang, Rockville, MD (US); Wenwei Huang, Rockville, MD (US); Ronald L. Johnson, Derwood, MD (US); Francesco Piscitelli, Marina Di Stongoli (IT)

(21) Appl. No.: 13/497,697

(22) PCT Filed: Sep. 22, 2010
(86) PCT No.: PCT/US10/49799

§ 371 Date: Sep. 13, 2012

Related U.S. Application Data
(60) Provisional application No. 61/245,045, filed on Sep. 23, 2009.
(60) Provisional application No. 61/309,554, filed on Mar. 2, 2010.

(51) Int. Cl. A61K 31/5025; C07D 495/04; A61K 31/5377; A61P 25/28; A61P 25/16

(52) U.S. Cl. 514/234.2; 514/248; 544/235; 544/117

(57) ABSTRACT

The present invention is directed to methods of inhibiting a tauopathy in a patient by administration of a compound of formula I:

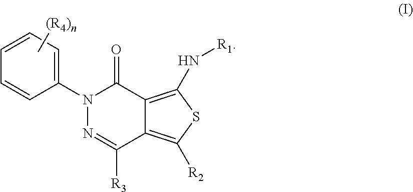

Novel aminothienopyridazine compounds are also described.

### CROSS REFERENCE TO RELATED APPLICATION

This application claims the benefit of U.S. Provisional Application Nos. 61/245,045, filed Sep. 23, 2009, and 61/309,554, filed Mar. 2, 2010, the entireties of which are incorporated herein.

### GOVERNMENT SUPPORT

This work was supported by grants from the National Institutes of Health (P01 AG09215, P30 AG10124, P01 AG11542, P01 AG14382, P01 AG14449, P01 AG17586, P01 AG19724, P01 NS-044233, U01 AG24904) and the NIH Roadmap for Medical Research and the Intramural Research Program of the National Human Genome Research Institute, National Institutes of Health. Pursuant to 35 U.S.C. §202, the government may have rights in any patent issuing from this application.

### TECHNICAL FIELD

The present invention is directed to aminothienopyradazine (ATPZ) compounds and their use for treating tauopathies.

### BACKGROUND

Inclusions comprised of fibrils of the microtubule (MT)-associated protein tau are found in the brains of those with Alzheimer's disease (AD), certain frontotemporal dementias and a host of additional neurodegenerative disorders that are broadly referred to as “tauopathies.” The pathology that is observed in these diseases is believed to result from the formation of toxic tau oligomers or fibrils, and/or from the loss of normal tau function due to its sequestration into insoluble deposits. Hence, small molecules that prevent tau oligomerization and/or fibrillization might have therapeutic value.

Despite the hypothesis that tau aggregation inhibitors may be therapeutically useful for AD and related tauopathies, to date only one compound of this type, methylene blue, has entered clinical trials. Numerous additional classes of compounds have been shown to inhibit tau assembly in vitro, but there are no reports of these molecules being evaluated in vivo for pharmacokinetic (PK) properties or efficacy in models of tauopathy. Because BBB permeability is known to be a major bottleneck that hampers the development of new CNS-active drugs, (Pardridge, W. M. The blood-brain barrier: bottleneck in brain drug development. NeuroRx 2005, 2, 3-14) an early evaluation of the brain penetration of candidate compounds is important, as such studies would permit focus on the most promising compound type.

### SUMMARY

Compounds and methods for the treatment of an amyloid disease or disorder in a patient are described. These methods comprise administering to the patient a therapeutically effective amount of a compound of formula I:

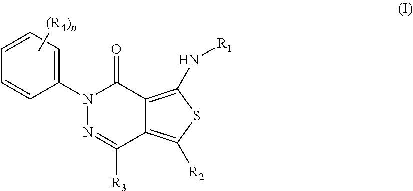

wherein:
R¹ is H, —C(O)—C₁₋₆alkyl; —C(O)—C₃₋₆cycloalkyl; —C(O)-aryl, —C(O)-heteroaryl; —C(O)alkaryl; —C(O)alkheteroaryl;
R² is H, halogen, C₁₋₆alkyl, or C₃₋₆cycloalkyl;
R³ is H, C₁₋₆alkyl; C₃₋₆cycloalkyl; aryl; heteroaryl; cycloalkyl; heterocycloalkyl; —C(O)—C₁₋₆alkyl; —C(O)—C₃₋₆cycloalkyl; —C(O)—O—C₁₋₆alkyl; —C(O)—O—C₃₋₆cycloalkyl; —NHC(O)OC₁₋₆alkyl; —NHC(O)OC₃₋₆cycloalkyl; —COOH; —C(O)NR^{3a}R^{3b} wherein R^{3a} and R^{3b} are each independently H; C₁₋₆alkylene-OH; —NH₂; C₁₋₆alkyl; C₃₋₆cycloalkyl; aryl; heteroaryl; alkaryl; alkheteroaryl; or R^{3a} and R^{3b}, together with the nitrogen to which they are attached, form a 5-, 6-, or 7-membered heterocycloalkyl;
n is 0, 1, 2, 3, 4, or 5;
each R⁴ is independently C₁₋₆alkyl optionally substituted with 1-3 halogen; C₃₋₆cycloalkyl optionally substituted with 1-3 halogen; halogen; C₁₋₆alkoxy optionally substituted with 1-3 halogen; —OH, —N₃; —NO₂; —NR^{4a}R^{4b} wherein R^{4a} and R^{4b} are each independently H, C₁₋₆alkyl; C₃₋₆cycloalkyl; aryl; heteroaryl; alkaryl; alkheteroaryl; or R^{3a} and R^{3b}, together with the nitrogen to which they are attached, form a 5-, 6-, or 7-membered heterocycloalkyl; aryl; heterocycloalkyl; heteroaryl, —C(O)—C₁₋₆alkyl, —C(O)—C₃₋₆cycloalkyl —C(O)-aryl; —C(O)O—C₁₋₆alkyl; —C(O)O—C₃₋₆cycloalkyl; or —C(O)—Oaryl; or a pharmaceutically acceptable salt form thereof.

### BRIEF DESCRIPTION OF THE DRAWINGS

FIG. 1. Effects of ATPZ tau fibrillization inhibitors on tau-mediated MT assembly and Aβ(1-42) fibril formation. (A) Representative traces of tau-induced microtubule assembly in the presence of 50 μM of an ATPZ or DMSO, as described in the Experimental Procedures. (B) Comparison of K18PL tau and Aβ(1-42) fibrillization in the presence of ATPZ's.

FIG. 2. Inhibition of tau assembly by 5b increases both monomeric and oligomeric tau content. Tau40 fibrillization reactions were performed in the presence or absence of 5b, and a non-fibrillizing control reaction was conducted with tau40 and 5b in the absence of heparin. Following incubations as described in Materials and Methods, the reaction mixtures underwent centrifugation and the resulting supernatant fractions were analyzed by SEC to determine the relative content of tau monomer and oligomer. The chromatographs shown correspond to the non-fibrillizing control (green) and fibrillization in the absence (red) or presence (black) of 5b.

FIG. 3. (A) Representative dose-response curves in the heparin-induced K18PL fibrillization assay for compounds E1 (CNDR-51348), F3 (CNDR-51349), and F4 (CNDR-51362) of the invention; (B) SDS-PAGE analysis of supernatant (S) or pellet (P) samples obtained after centrifugation of fibrillizing mixtures incubated in the presence of DMSO vehicle, or 100 μM of test compounds E1 (CNDR-51348), F3 (CNDR-51349), and F4 (CNDR-51362).

FIG. 4. (A) Comparison of maximal percent inhibition of K18PL fibrillization caused by test compounds in which the fibrillizing mixtures did (sup) or did not (tot) undergo centrifugation at time zero to remove insoluble material. Compounds 17, E1, F3, F5, F4, F2, F6, F1, F7, 2 were added at 50 μM to the tau fibrillization mixture and incubated for 30 min. Samples were then divided in two halves, one of which was incubated without further treatment (“tot”), and one that was centrifuged at 186,000 g for 30 min. After centrifugation of the latter sample, the supernatants (“sup”) was collected and incubated as per the fibrillization protocol previously reported in Crowe, A, et al. Identification of aminothienopyridazine inhibitors of tau assembly by quantitative high-throughput screening. Biochemistry 2009, 48, 7732-45 (B) SDS-PAGE analysis of supernatant (S) or pellet (P) samples for E1 (CNDR-51348) and F3 (CNDR-51349) obtained after centrifugation of fibrillizing mixtures prior to incubation; (C) Representative dose-response curves with (“sup”) and without (“tot”) initial centrifugation for E1 (CNDR-51348) and F3 (CNDR-51349).

FIG. 5. MT-Polymerization assay. Tubulin (50 μM) and tau40 (30 μM) were incubated with ATPZ F3 (CNDR-51349) (solid circles) or F4 (CNDR-51362) (triangles) or DMSO (open and black squares) to evaluate possible interference in the tau-promoted MT-polymerization that may be caused by the'ATPZ inhibitors of tau aggregation. As control experiment, MT-polymerization assay was also conducted in the absence of tau40 (open circles).

FIG. 6. (A) Brain and plasma level of F4 (CNDR-51362) after i.v. administration at 2 mg/Kg; (B) Brain and plasma level of F4 (CNDR-51362) after oral administration at 5 mg/Kg; (C) Brain and plasma level of F3 (CNDR-51349) after oral administration at 5 mg/Kg

### DETAILED DESCRIPTION OF ILLUSTRATIVE EMBODIMENTS

The present invention is directed to methods of treating a tauopathy in a patient. Tauopathies are a class of neurodegenerative diseases resulting from the pathological aggregation of tau protein. Examples of tauopathies include, but are not limited to, frontotemporal dementia, Alzheimer's disease, progressive supranuclear palsy, corticobasal degeneration, and frontotemporal lobar degeneration (Pick's disease). In preferred embodiments, the tauopathy is Alzheimer's disease.

The invention is directed to methods of treating a tauopathy in a patient comprising administering to the patient a therapeutically effective amount of a compound of formula I:

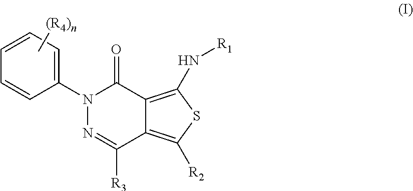

wherein:
R¹ is H, —C(O)—C₁₋₆alkyl; —C(O)—C₃₋₆cycloalkyl; —C(O)-aryl, —C(O)-heteroaryl; —C(O)alkaryl; —C(O)alkheteroaryl;
R² is H, halogen, C₁₋₆alkyl, or C₃₋₆cycloalkyl;
R³ is H, C₁₋₆alkyl; C₃₋₄cycloalkyl; C₁₋₆alkyl-OH; aryl; heteroaryl; cycloalkyl; heterocycloalkyl; —C(O)—C₁₋₆alkyl; —C(O)—C₃₋₆cycloalkyl; —C(O)—O—C₁₋₆alkyl; —C(O)—O—C₃₋₆cycloalkyl; —NHC(O)OC₁₋₆alkyl; —NHC(O)OC₃₋₆cycloalkyl; —COOH; —C(O)NR^{3a}R^{3b} wherein R^{3a} and R^{3b} are each independently H; —NH₂; C₁₋₆alkylene-OH; C₁₋₆alkyl; C₃₋₆cycloalkyl; aryl; heteroaryl; alkaryl; alkheteroaryl; or R^{3a} and R^{3b}, together with the nitrogen to which they are attached, form a 5-, 6-, or 7-membered heterocycloalkyl;
n is 0, 1, 2, 3, 4, or 5;
each R⁴ is independently C₁₋₆alkyl optionally substituted with 1-3 halogen; C₃₋₆cycloalkyl optionally substituted with 1-3 halogen; halogen; C₁₋₆alkoxy optionally substituted with 1-3 halogen; —OH, —N₃; —NO₂; —NR^{4a}R^{4b} wherein R^{4a} and R^{4b} are each independently H, C₁₋₆alkyl; C₃₋₆cycloalkyl; aryl; heteroaryl; alkaryl; alkheteroaryl; or R^{3a} and R^{3b}, together with the nitrogen to which they are attached, form a 5-, 6-, or 7-membered heterocycloalkyl; aryl; heterocycloalkyl; heteroaryl, —C(O)—C₁₋₆alkyl, —C(O)—C₃₋₆cycloalkyl —C(O)-aryl; —C(O)O—C₁₋₆alkyl; —C(O)O—C₃₋₆cycloalkyl; or —C(O)—Oaryl; or a pharmaceutically acceptable salt forms thereof.

Also within the scope of the invention are methods of treating a tauopathy in a patient comprising administering to the patient a therapeutically effective amount of a compound of formula I, wherein:
R¹ is H, —C(O)—C₁₋₆alkyl; —C(O)—C₃₋₆cycloalkyl —C(O)-aryl, —C(O)-heteroaryl; —C(O)alkaryl; —C(O)alkheteroaryl;
R² is H, C₁₋₆alkyl, or —C₃₋₆cycloalkyl;
R³ is H, C₁₋₆alkyl; C₁₋₆alkyl-OH; aryl; heteroaryl; cycloalkyl; heterocycloalkyl; —C(O)—C₁₋₆alkyl; —C(O)—C₃₋₆cycloalkyl —COOH; —C(O)NR^{3a}R^{3b} wherein R^{3a} and R^{3b} are each independently H; C₁₋₆alkyl; —C₃₋₆cycloalkyl; aryl; heteroaryl; alkaryl; alkheteroaryl; or R^{3a} and R^{3b}, together with the nitrogen to which they are attached, form a 5-, 6-, or 7-membered heterocycloalkyl;
n is 0, 1, 2, 3, 4, or 5; and
each R⁴ is independently C₁₋₆alkyl optionally substituted with 1-3 halogen; C₃₋₆cycloalkyl optionally substituted with 1-3 halogen, halogen; C₁₋₆alkoxy; —OH, heterocycloalkyl; heteroaryl, —C(O)—C₁₋₆alkyl, —C(O)-aryl; —C(O)O—C₁₋₆alkyl; —C(O)O—C₃₋₆cycloalkyl-C(O)—Oaryl; or a pharmaceutically acceptable salt form thereof.

Also within the scope of the invention are methods of treating a tauopathy in a patient comprising administering to the patient a therapeutically effective amount of a compound of formula I, wherein:
R¹ is H, —C(O)—C₁₋₆alkyl; —C(O)—C₃₋₆cycloalkyl; —C(O)-aryl, —C(O)-heteroaryl; —C(O)alkaryl; —C(O)alkheteroaryl;
R² is H, C₁₋₆alkyl, or C₃₋₆cycloalkyl;
R³ is H, C₁₋₆alkyl; C₃₋₆cycloalkyl; C₁₋₆alkyl-OH; aryl; heteroaryl; cycloalkyl; heterocycloalkyl; —C(O)—C₁₋₆alkyl; —C(O)—C₃₋₆cycloalkyl; —C(O)—O—C₁₋₆alkyl; —C(O)—O—C₃₋₆cycloalkyl; —COOH; —C(O)NR^{3a}R^{3b} wherein R^{3a} and R^{3b} are each independently H; C₁₋₆alkyl; C₃₋₆cycloalkyl; aryl; heteroaryl; alkaryl; alkheteroaryl; or R^{3a} and R^{3b}, together with the nitrogen to which they are attached, form a 5-, 6-, or 7-membered heterocycloalkyl;
n is 0, 1, 2, 3, 4, or 5; and
each R⁴ is independently C₁₋₆alkyl optionally substituted with 1-3 halogen; C₃₋₆cycloalkyl optionally substituted with 1-3 halogen; halogen; C₁₋₆alkoxy; —OH; heterocycloalkyl; heteroaryl, —C(O)—C₁₋₆alkyl, —C(O)—C₃₋₆cycloalkyl —C(O)-aryl; —C(O)O—C₁₋₆alkyl; —C(O)O—C₃₋₆cycloalkyl; or —C(O)—Oaryl.

Also within the scope of the invention are methods of treating a tauopathy in a patient comprising administering to the patient a therapeutically effective amount of a compound of formula I, wherein:
R¹ is H, —C(O)—C₁₋₆alkyl; —C(O)-aryl, —C(O)-heteroaryl; —C(O)alkaryl; —C(O)alkheteroaryl;
R² is H or C₁₋₆alkyl;
R³ is H, C₁₋₆alkyl; C₁₋₆alkyl-OH; aryl; heteroaryl; cycloalkyl; heterocycloalkyl; —C(O)—C₁₋₆alkyl; —COOH; —C(O)NR^{3a}R^{3b} wherein R^{3a} and R^{3b} are each independently H; C₁₋₆alkyl; aryl; heteroaryl; alkaryl; alkheteroaryl; or R^{3a} and R^{3b}, together with the nitrogen to which they are attached, form a 5-, 6-, or 7-membered heterocycloalkyl; and
   each R⁴ is independently C₁₋₆alkyl optionally substituted with 1-3 halogen; halogen; C₁₋₆alkoxy; —OH, heterocycloalkyl; heteroaryl, —C(O)—C₁₋₆alkyl, —C(O)-aryl; —C(O)O—C₁₋₆alkyl; or —C(O)—Oaryl.

In preferred embodiments, R¹ is H. In other embodiments, R¹ is C₁₋₆alkyl, for example, —CH₃. In other embodiments, R¹ is —C(O)C₁₋₆alkyl, for example, acetyl [—C(O)CH₃]. In yet other embodiments, R¹ is —C(O)aryl, for example, —C(O)-phenyl. In other embodiments, R¹ is —C(O)alkaryl, for example, —C(O)CH₂C₆H₅.

In other embodiments, R² is H. In other embodiments, R² is halogen. Preferred halogens for R² are bromine and chlorine. In yet other embodiments, R² is C₁₋₆alkyl, for example, —CH₃. In still other embodiments, R² is C₃₋₆cycloalkyl, for example, cyclopropyl.

In some embodiments, R³ is C₁₋₆alkyl, for example, —CH₃ or —CH₂CH₃.

In some embodiments, R³ is aryl, for example, phenyl.

In preferred embodiments, R³ is —C(O)—O—C₁₋₆alkyl, for example, —C(O)—O—CH₂CH₃ or —C(O)—O—CH₃.

In other preferred embodiments, R³ is —COOH.

In other embodiment, R³ is C₁₋₆alkylene-OH.

In some embodiments, R³ is —NHC(O)OC₁₋₆alkyl. Preferably R³ is —NHC(O)OCH₂CH₃ or —NHC(O)Ot-butyl. In other embodiments, R³ is —NHC(O)OC₃₋₆cycloalkyl, for example, —NHC(O)Ocyclopropyl.

In other embodiments, R³ is —NH₂.

In yet other embodiments of the invention, R³ is —C(O)NR^{3a}R^{3b}. Preferably, R^{3a} and R^{3b} are each H or R^{3a} and R^{3b} are each independently C₁₋₆alkyl, for example —CH₃, —CH₂CH₃, or isopropyl. In other embodiments R^{3a} is C₁₋₆alkyl, for example, —CH₃, —CH₂CH₃, or isopropyl, and R^{3b} is C₃₋₆cycloalkyl, for example, cyclopropyl.

In other embodiments, R^{3a} is H. In other embodiments, R^{3b} is H and R^{3a} is C₁₋₆alkyl, for example, —CH₃, —CH₂CH₃, and isopropyl. Also preferred is wherein R^{3b} is H and R^{3a} is C₃₋₆cycloalkyl, for example cyclopropyl. Also preferred is wherein R^{3b} is H and R^{3a} is alkaryl, for example, benzyl. Also preferred is wherein R^{3b} is H and R^{3a} is —NH₂. Also preferred is wherein R^{3b} is H and R^{3a} is —C₁₋₆alkylene-OH, for example, —CH₂CH₂—OH.

In other embodiments, R^{3a} and R^{3b}, together with the nitrogen to which they are attached, form a 5-, 6-, or 7-membered heterocycloalkyl. Preferably, the heterocycloalkyl is piperidyl or morpholinyl.

In preferred embodiments, the compounds of formula I include one R⁴ substituent. In those embodiments, it is preferred that the R⁴ substituent is at the 4-position of the ring. In other embodiments, the R⁴ substituent is at the 3-position of the ring. In other embodiments, R⁴ substituent is at the 2-position of the ring.

In other embodiments, the compounds of formula I include two R⁴ substituents. In those embodiments, it is preferred that the R⁴ substituents are at the 2 and 3 positions of the ring. In other embodiments, the R⁴ substituents are at the 3 and 4 positions of the ring. In other embodiments, the R⁴ substituents are at the 2 and 4 positions of the ring.

A preferred substitutent for R⁴ is hydrogen. Other preferred substituents for R⁴ include halogen, for example, —F, —Cl, —Br, and —I, preferably, —F, —Cl, and —Br. Yet other preferred substituents for R⁴ are C₁₋₆alkyl, for example, —CH₃, —CH₂CH₃, and isopropyl. Other preferred substituents for R⁴ are C₁₋₆alkyl substituted with 1-3 halogen, for example, —CF₃. Other preferred substituents for R⁴ are C₁₋₆alkoxy, for example, —OCH₃. Other preferred substituents for R⁴ are C₁₋₆alkoxy substituted with 1-3 halogen, for example, —OCF₃.

Another preferred substituted for R⁴ is aryl, for example, phenyl.

Other preferred substituents for R⁴ are —C(O)O—C₁₋₆alkyl, for example, —C(O)O—CH₃, —C(O)O—CH₂CH₃, and —C(O)O-isopropyl.

Another preferred substituent for R⁴ is —OH. Yet another preferred substitutent for R⁴ is —NO₂. Still another preferred substituent for R⁴ is —N₃.

Still other preferred embodiments are where R⁴ is —NR^{4a}R^{4b}. In some embodiments, R^{4a} and R^{4b} are each H. In other embodiments, R^{4a} and R^{4b} are each independently C₁₋₆alkyl, for example —CH₃, —CH₂CH₃, and isopropyl. In other embodiments R^{4a} is hydrogen and R^{4b} is C₁₋₆alkyl, for example, —CH₂, —CH₂CH₃, and isopropyl.

Preferred compounds for use in the methods of the present invention are set forth in Table 1:

TABLE 1

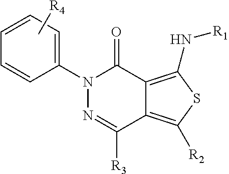

| Comp No. | R1 | R2 | R3 | R4 |
|---|---|---|---|---|
| 5b | H | H | —COOEt | 4-OMe |
| 9a | 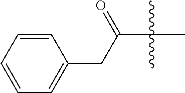 [SMILES: *C(=O)Cc1ccccc1] | H | —COOEt | 4-OMe |
| 9b | Ac— | H | —COOEt | H |
| 9c | 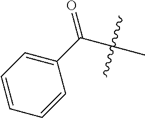 [SMILES: *C(=O)c1ccccc1] | H | —COOEt | H |
| 16 | Ac | Me | H | H |
| 5f | H | Me | —COOEt | H |
| 8d | H | H | 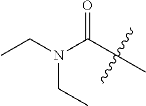 [SMILES: *C(=O)N(CC)CC] | H |
| 15 | H | H | 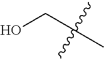 [SMILES: *CO] | H |
| 8c | H | H | 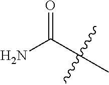 [SMILES: *C(N)=O] | H |
| 8a | H | H | 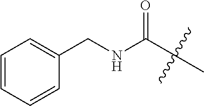 [SMILES: *C(=O)NCc1ccccc1] | H |
| 8b | H | H | 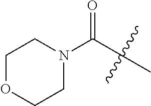 [SMILES: *C(=O)N1CCOCC1] | H |
| 14 | H | H | —NH₂ | H |
| 6a | H | H | —COOH | H |
| 6b | H | H | —COOH | 4-OMe |
| 5a | H | H | —COOEt | H |
| 17 | H | H | —COOEt | 4-Cl |
| 5g | H | H | —COOEt | 3,4-dichloro |
| 5h | H | H | —COOEt | 4-Me |
| 5c | H | H | —COOEt | 3,4-dimethyl |
| 18 | H | H | —COOMe | 4-F |
| 5d | H | H | —COOEt | 2-F |
| 19 | H | H | —COOMe | 3-CF₃ |
| 5e | H | H | —COOEt | 4-COOEt |
| CNDR-51371 | H | H | —COOiPr | 4-Cl |
| (2) | | | | |
| CNDR-51367 | H | H | —CONHMe | 4-Cl |
| (F1) | | | | |
| CNDR-51365 | H | H | —CONHEt | 4-Cl |
| (F2) | | | | |
| CNDR-51349 | H | H | —CONHiPr | 4-Cl |
| (F3) | | | | |
| CNDR-51362 | H | H | —CONH—c-Pr | 4-Cl |
| (F4) | | | | |
| CNDR-51361 | H | H | —CON(Me)₂ | 4-Cl |
| (F5) | | | | |
| CNDR-51366 | H | H | —CON(Me)Et | 4-Cl |
| (F6) | | | | |
| CNDR-51390 | H | H | —CONHiPr | 4-Br |
| (F7) | | | | |
| CNDR-51386 | H | H | —COOEt | 4-Br |
| (D2) | | | | |
| CNDR-51348 | H | H | —COOH | 4-Cl |
| (E1) | | | | |
| CNDR-51389 | H | H | —COOH | 4-Br |
| (E2) | | | | |
| CNDR-51443 | H | H | —C(O)N(Et)₂ | 4-OCH₃ |
| (F10) | | | | |
| CNDR-51394 | H | H | —COOEt | 4-F |
| (D3) | | | | |
| CNDR-51363 | H | H | —COOEt | 4-OH |
| (D11) | | | | |
| CNDR-51423 | H | H | —COOEt | 3-CH₃ |
| (D15) | | | | |
| CNDR-51404 | H | H | —COOEt | 4-I |
| (D4) | | | | |
| CNDR-51439 | H | H | —COOEt | 2-F |
| CNDR-51352 | H | H | —COOEt | 4-NO₂ |
| (D5) | | | | |
| CNDR-51373 | H | H | —COOEt | 4-CF₃ |
| (D12) | | | | |
| CNDR-51387 | H | H | —COOEt | 3-Cl |
| CNDR-51425 | H | H | —COOEt | 2-Cl |
| (D19) | | | | |
| CNDR-51369 | H | H | —COOEt | 4-OCF₃ |
| (D10) | | | | |
| CNDR-51424 | H | H | —COOEt | 2-CH₃ |
| (D23) | | | | |
| CNDR-51347 | H | H | —COOEt | 4-iPr |
| (D13) | | | | |

Preferred compounds for use in the methods of the present invention are set forth in Table 2:

TABLE 2

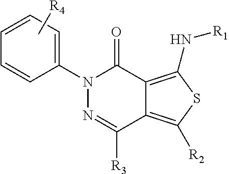

| Comp No. | R1 | R2 | R3 | R4 |
|---|---|---|---|---|
| 5f | H | Me | —COOEt | H |
| 8d | H | H | 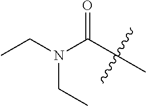 [SMILES: *C(=O)N(CC)CC] | 4-OCH₃ |
| 8c | H | H | 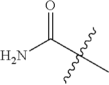 [SMILES: *C(N)=O] | H |
| 8a | H | H | 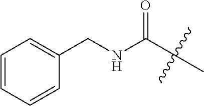 [SMILES: *C(=O)NCc1ccccc1] | H |
| 8b | H | H | 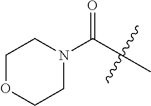 [SMILES: *C(=O)N1CCOCC1] | H |
| 14 | H | H | —NH₂ | H |
| 5g | H | H | —COOEt | 3,4-dichloro |
| 18 | H | H | —COOMe | 4-F |
| 5d | H | H | —COOEt | 2-F |
| 19 | H | H | —COOMe | 3-CF₃ |
| 5e | H | H | —COOEt | 4-COOEt |
| CNDR-51367 | H | H | —CONHMe | 4-Cl |
| (F1) | | | | |
| CNDR-51365 | H | H | —CONHEt | 4-Cl |
| (F2) | | | | |
| CNDR-51349 | H | H | —CONHiPr | 4-Cl |
| (F3) | | | | |
| CNDR-51362 | H | H | —CONH—c-Pr | 4-Cl |
| (F4) | | | | |
| CNDR-51361 | H | H | —CON(Me)₂ | 4-Cl |
| (F5) | | | | |
| CNDR-51366 | H | H | —CON(Me)Et | 4-Cl |
| (F6) | | | | |
| CNDR-51390 | H | H | —CONHiPr | 4-Br |
| (F7) | | | | |
| CNDR-51386 | H | H | —COOEt | 4-Br |
| (D2) | | | | |
| CNDR-51348 | H | H | —COOH | 4-Cl |
| (E1) | | | | |
| CNDR-51389 | H | H | —COOH | 4-Br |
| (E2) | | | | |
| CNDR-51395 | H | H | —COOH | 4-F |
| (E3) | | | | |
| CNDR-51374 | H | H | —COOH | 4-OH |
| (E4) | | | | |
| CNDR-51388 | H | H | —COOH | 3-Cl |
| (E6) | | | | |
| CNDR-51370 | H | H | —COOH | 4-OCF₃ |
| (E5) | | | | |
| CNDR-51405 | H | H | —Ph | 4-Cl |
| CNDR-51397 | H | H | —C(O)NH—cPr | 4-F |
| (F9) | | | | |
| CNDR-51355 | H | H | —C(O)OEt | 4-NH₂ |
| (D7) | | | | |
| CNDR-51422 | H | H | —C(O)OEt | 3-Br |
| (D14) | | | | |
| CNDR-51411 | H | Br | —C(O)OEt | 4-Cl |
| CNDR-51393 | H | Cl | —C(O)OEt | 4-Cl |
| CNDR-51412 | H | H | —C(O)OEt | 4-N₃ |
| CNDR-51358 | H | H | —C(O)OEt | 4-NHCH₃ |
| (D8) | | | | |
| CNDR-51359 | H | H | —C(O)OEt | 4-N(CH₃)₂ |
| (D9) | | | | |
| CNDR-51421 | H | H | —C(O)OEt | 2-Br |
| (D20) | | | | |
| CNDR-51434 | H | H | —C(O)OEt | 2,4, |
| (D24) | | | | dimethyl |
| CNDR-51420 | H | H | —C(O)OEt | 3-C(O)OEt |
| (D17) | | | | |
| CNDR-51419 | H | H | —C(O)OEt | 2-C(O)OEt |
| (D21) | | | | |
| CNDR-51435 | H | H | —C(O)OEt | 2-Ph |
| (D22) | | | | |
| CNDR-51385 | H | H | —NHC(O)OEt | 4-Cl |
| CNDR-51391 | H | H | —C(O)NH—iPr | 3-Cl |
| CNDR-51396 | H | H | —C(O)NH—iPr | 4-F |
| (F8) | | | | |
| CNDR-51376 | H | H | —C(O)NH—iPr | 4-OH |
| (F13) | | | | |
| CNDR-51375 | H | H | —C(O)NH—iPr | 4-OCF₃ |
| (F12) | | | | |
| CNDR-51400 | H | H | —CH₃ | 4-Cl |
| CNDR-51378 | H | H | —C(O)NHNH₂ | 4-Cl |
| CNDR-51433 | H | H | —C(O)NHCH₂CH₂OH | 4-Cl |
| (F14) | | | | |
| CNDR-51383 | H | H | —NHC(O)OtBu | 4-Cl |
| CNDR-51360 | CH₃ | H | —C(O)OEt | 4-Cl |

The compounds set forth in Table 2 are also within the scope of the invention.

As used herein, the term “alkyl” refers to a straight-chain, or branched alkyl group having 1 to 8 carbon atoms, such as methyl, ethyl, propyl, isopropyl, butyl, isobutyl, sec-butyl, tert-butyl, pentyl, isoamyl, neopentyl, 1-ethylpropyl, 3-methylpentyl, 2,2-dimethylbutyl, 2,3-dimethylbutyl, hexyl, octyl, etc. The alkyl moiety of alkyl-containing groups has the same meaning as alkyl defined above. A designation such as “C₁-C₆ alkyl” refers to straight-chain, or branched alkyl group having 1 to 6 carbon atoms, such as methyl, ethyl, propyl, isopropyl, butyl, isobutyl, sec-butyl, tert-butyl, pentyl, isoamyl, neopentyl, 1-ethylpropyl, 3-methylpentyl, 2,2-dimethylbutyl, 2,3-dimethylbutyl, hexyl, etc. Lower alkyl groups, which are preferred, are alkyl groups as defined above which contain 1 to 4 carbons. A designation such as “C₁-C₄ alkyl” refers to an alkyl radical containing from 1 to 4 carbon atoms, such as methyl, ethyl, propyl, isopropyl, butyl, isobutyl, sec-butyl, and tert-butyl. A designation such as “C₁-C₃ alkyl” refers to an alkyl radical containing from 1 to 3 carbon atoms, such as methyl, ethyl, propyl, and isopropyl.

As used herein, the term “alkylene” refers to a divalent radical derived from an alkane, for example, —CH₂CH₂CH₂—. A designation such as “C₁-C₆ alkylene” refers to straight-chain alkylene group having 1 to 6 carbon atoms, such as methylene, ethylene, propylene, butylene, and the like.

As used herein, the term “cycloalkyl” refers to a saturated or partially saturated mono- or bicyclic alkyl ring system containing 3 to 11 carbon atoms. Certain embodiments contain 3 to 10 carbon atoms, other embodiments contain 3 to 7 carbon atoms, other embodiments contain 3 to 6 carbon atoms, and other embodiments contain 5 or 6 carbon atoms. A designation such as “C₃-C₇ cycloalkyl” refers to a cycloalkyl radical containing from 3 to 7 ring carbon atoms. Examples of cycloalkyl groups include such groups as cyclopropyl, cyclobutyl, cyclopentyl, cyclohexyl, cyclohexenyl, and cycloheptyl.

As used herein, the term “aryl” refers to a substituted or unsubstituted, mono- or bicyclic hydrocarbon aromatic ring system having 6 to 10 ring carbon atoms. Examples include phenyl and naphthyl. As used herein, the term “alkaryl” refers to an alkylene-aryl group, such as, for example, benzyl.

As used herein, the term “heteroaryl” refers to an aromatic group or ring system containing 5 to 10 ring carbon atoms in which one or more ring carbon atoms are replaced by at least one hetero atom such as O, N, or S. Certain embodiments include 5 or 6 membered rings. Examples of heteroaryl groups include pyrrolyl, furanyl, thienyl, pyrazolyl, imidazolyl, thiazolyl, isothiazolyl, isoxazolyl, oxazolyl, oxathiolyl, oxadiazolyl, triazolyl, oxatriazolyl, furazanyl, tetrazolyl, pyridyl, pyrazinyl, pyrimidinyl, pyridazinyl, triazinyl, picolinyl, imidazopyridinyl, indolyl, isoindolyl, indazolyl, benzofuranyl, isobenzofuranyl, purinyl, quinazolinyl, quinolyl, isoquinolyl, benzoimidazolyl, benzothiazolyl, benzothiophenyl, thianaphthenyl, benzoxazolyl, benzooxadiazolyl, benzisoxazolyl, cinnolinyl, phthalazinyl, naphthyridinyl, and quinoxalinyl. As used herein, the term “alkheteroaryl” refers to an alkylene-heteroaryl group, for example —CH₂-heteroaryl.

As used herein, the term “heterocycloalkyl” refers to a cycloalkyl group in which one or more ring carbon atoms are replaced by at least one hetero atom such as O, N, S, SO, and SO₂. Certain embodiments include 3 to 6 membered rings, and other embodiments include 5 or 6 membered rings. Examples of heterocycloalkyl groups include azetidinyl, 3H-benzooxazolyl, 1,1-dioxo-thiomorpholinyl, 1,4-diazapinyl, pyrrolidinyl, pyrrolinyl, imidazolidinyl, imidazolinyl, imidazolidinyl, oxazolidinyl, pirazolidinyl, pirazolinyl, pyrazalinyl, piperidinyl, piperazinyl, hexahydropyrimidinyl, morpholinyl, thiomorpholinyl, dihydrobenzofuranyl, tetrahydrofuranyl, tetrahydropyranyl, tetrahydropyrazolopyridinyl, tetrahydro-1,3a,7-triaza-azulenyl, dihydrooxazolyl, dithiolyl, oxathiolyl, dioxazolyl, oxathiazolyl, pyranyl, oxazinyl, oxathiazinyl, and oxadiazinyl. Included within the definition of “heterocycloalkyl” are fused ring systems, including, for example, ring systems in which an aromatic ring is fused to a heterocycloalkyl ring and ring systems in which a heteroaromatic ring is fused to a cycloalkyl ring or a heterocycloalkyl ring. Examples of such fused ring systems include, for example, 2,3-dihydrobenzofuran, 2,3-dihydro-1,3-benzoxazole, phthalamide, phthalic anhydride, indoline, isoindoline, tetrahydroisoquinoline, chroman, isochroman, chromene, and isochromene.

As used herein “halogen” refers to fluorine, chlorine, bromine, and iodine.

A “therapeutically effective amount” (or dose) is an amount that, upon administration to a patient, results in a discernible patient benefit (e.g., provides detectable relief from at least one condition being treated). It will be apparent that the discernible patient benefit may be apparent after administration of a single dose, or may become apparent following repeated administration of the therapeutically effective dose according to a predetermined regimen, depending upon the indication for which the compound is administered.

As used herein, “pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problem complications commensurate with a reasonable benefit/risk ratio.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like. These physiologically acceptable salts are prepared by methods known in the art, e.g., by dissolving the free amine bases with an excess of the acid in aqueous alcohol, or neutralizing a free carboxylic acid with an alkali metal base such as a hydroxide, or with an amine.

As used herein, “patient” refers to animals, including mammals, preferably humans.

The terms “treatment” and “treating” as used herein include preventative (e.g., prophylactic), curative and/or palliative treatment.

### Experimental Procedures

### Tau Protein Preparations

Full length tau (Tau40; amino acids 1-441) (Goedert, M., Spillantini, M. G., Potier, M. C., Ulrich, J., and Crowther, R. A. (1989) Embo Journal 8, 393-399), tau K18 fragment containing the P301L missense mutation (Goedert, M. (2005) Movement Disorders 20, S45-S52) (K18PL; amino acids 244-369) (Gustke, N., Trinczek, B., Biernat, J., Mandelkow, E. M., and Mandelkow, E. (1994) Biochemistry 33, 9511-9522), and a non-fibrillizing K18 tau construct carrying the K311D missense mutation (K18KD) (Li, W. K. and Lee, V. M. Y. (2006) Biochemistry 45, 15692-15701) were cloned into the pRK172 expression vector, expressed and purified as previously described (Hong, M., Zhukareva, V., Vogelsberg-Ragaglia, V., Wszolek, Z., Reed, L., Miller, B. I., Geschwind, D. H., Bird, T. D., McKeel, D., Goate, A., Morris, J. C., Wilhelmsen, K. C., Schellenberg, G. D., Trojanowski, J. Q., and Lee, V. M. Y. (1998) Science 282, 1914-1917). To eliminate batch-to-batch variations that might contribute to assay variability, K18PL from several preparations was pooled so that a single homogeneous preparation was used throughout qHTS.

### Alexa Red Labeling of K18PL and K18KD

K18PL and K18 KD were labeled with Alexa Red (Invitrogen) according to the manufacturer's protocol. Labeled protein was determined to contain 1 mol of dye per 3 mol protein, following the quantification method described in the manufacturer's instructions.

### Fibrillization Reactions

Tau fibril formation reactions were performed essentially as described (Crowe, A., Ballatore, C., Hyde, E., Trojanowski, J. Q., and Lee, V. M. Y. (2007) Biochemical and Biophysical Research Communications 358, 1-6). K18PL (2 μl of a 30 μM stock that also contained 0.24 μM Alexa Red-labeled K18PL) was dispensed into black 1536-well plates (Grenier) using a solenoid-based dispenser. Following transfer of 23 nL of test compound or DMSO vehicle by a pin tool from compound library plates, 2 μl/well of 40 μM heparin in reagent buffer was dispensed and the plate was centrifuged for 15 s at 1000 rpm in a table-top centrifuge. The fibrillization reaction proceeded for 6 hours at 37° C. in a humidified incubator (Kalypsys).

### ThT Assay

K18PL fibril formation was quantified with a ThT assay as previously described (Crowe, A., Ballatore, C., Hyde, E., Trojanowski, J. Q., and Lee, V. M. Y. (2007) Biochemical and Biophysical Research Communications 358, 1-6). Briefly, 1 μl/well of 62.5 μM ThT (Sigma) in 100 mM glycine, pH 8.5 (12.5 μM final concentration) was dispensed into 1536-well plates after completion of the fibrillization reaction and incubated at room temperature for 1 hour. Plates were read on an Envision fluorescence plate reader with an excitation of 450 nm and an emission setting of 510 nm.

### Tau Fluorescence Polarization (FP) Assay

A tau FP method was developed to quantify Alexa Red-labeled K18PL tau incorporation into multimeric tau assemblies based on a similar methodology that was previously employed to measure the oligomerization and fibrillization of α-synuclein (Luk, K. C., Hyde, E. G., Trojanowski, J. Q., and Lee, V. M. Y. (2007) Biochemistry 46, 12522-12529). Although Alexa Red-labeled K18PL tau preparations did not readily assemble into fibrils under the conditions described above, the fluorescently-labeled tau could be incorporated into nascent oligomers and fibrils with normal assembly kinetics when mixed with unlabeled K18PL at a 1:62.5 molar ratio. Under these conditions, a fluorescence polarization increase of >100 polarization units (mP) was obtained upon completion of the fibrillization reaction, as determined on an Envision fluorescence plate reader using a Texas Red FP dual mirror, Texas Red FP excitation filter at 555 nm, and Texas Red FP emission filter (P and S channel) at 632 nm. The detector gains for each emission filter were calibrated to obtain maximum mP values. FP values were calculated from the S and P channel readings according to the equation, mP=1000*(S−G*P)/(S+G*P), with the G value set to one. Total Alexa Red fluorescence emission was calculated using the equation TF=S+2*P.

### Tau Assembly Inhibitor Data Analysis

Compound effects on tau assembly were determined for the ThT and FP assays using the formula 1−(F_{compound}− F̅_{DMSO})/( F̅_{control}− F̅_{DMSO}), where F̅_{control} denotes the median plate fluorescence values (ThT assay) or mP values (FP assay) of the non-fibrillizing K18 KD control wells, F̅_{DMSO} denotes the median value of the DMSO control wells, and F_{compound} denotes the compound well values. The resulting values were multiplied by 100 to obtain the percent inhibition of tau fibril formation.

### Centrifugation (Sedimentation) and Electron Microscopy (EM) Assays

The determination of compound-induced effects on the amount of tau that remains soluble upon centrifugation, and EM visualization of tau species following incubation with test compounds, were as previously described (Crowe, A., Ballatore, C., Hyde, E., Trojanowski, J. Q., and Lee, V. M. Y. (2007) Biochemical and Biophysical Research Communications 358, 1-6).

### MT Assembly Assay

The effect of compounds on tau-mediated MT assembly was essentially as described (Hong, M., Zhukareva, V., Vogelsberg-Ragaglia, V., Wszolek, Z., Reed, L., Miller, B. I., Geschwind, D. H., Bird, T. D., McKeel, D., Goate, A., Morris, J. C., Wilhelmsen, K. C., Schellenberg, G. D., Trojanowski, J. Q., and Lee, V. M. Y. (1998) Science 282, 1914-1917), adapted to a 384-well plate. Wild-type K18 tau was used for MT assembly because K18PL has a decreased ability to promote MT assembly. Lyophilized bovine brain tubulin (Cytoskeleton Inc.) was reconstituted in RAB (100 mM MES, pH 6.9; 1 mM EDTA; 0.5 mM MgSO₄) at a concentration of 10 mg/ml. Compounds (50 μM) were added to K18 tau (40 μM) in RAB and pre-incubated at room temperature for 60 minutes. To initiate the MT assembly reaction, 8.25 μl per well of 10 mg/ml tubulin was dispensed on a UV-clear 384-well plate (Corning #3675), followed by 1.0 μl of 100 mM GTP in RAB and 41.25 μl of the compound:tau mixture. This resulted in a final reaction mixture of 30 μM tubulin, 40 μM K18, 50 μM compound and 2 mM GTP. The plate was incubated in a Spectramax M5 plate reader at 37° C. and the absorbance at 340 nm was read every minute for 45 minutes.

### Aβ₁₋₄₂ Fibrillization Assay

Synthetic Aβ(1-42) peptide (rPeptide) was resuspended in 1,1,1,3,3,3-hexafluoroisopropanol (HFIP) at a concentration of 5 mg/ml for 30 minutes and then air dried in small aliquots, followed by storage at −80° C. For fibrillization assays (46), HFIP-treated Aβ(1-42) aliquots were reconstituted to 2 mg/ml in DMSO and then diluted to 15 μM in 25 mM Tris, pH 7.0 buffer to which test compound was added at 50 μM final concentration, or at several concentrations ranging from 0.16-40 μM. The reaction mixtures were dispensed at 25 μl/well into a 384-well plate (NUNC #262260) and then incubated at 37° C. for 4 hrs. Upon completion of the reaction, 25 μl of 25 μM ThT was added to each well followed by ThT fluorescence readings as described above for K18PL.

### Size-Exclusion Chromatography (SEC) of Tau40 Fibrillization Reaction Post-Centrifugation Supernatants

Tau40 (50 μM) and heparin (20 μM) were incubated in a PCR tube with or without the compound 5b (100 μM) at 37° C. for 5 days in a final volume of 100 μl of 100 in M sodium acetate buffer, pH 7.0. In the absence of compound, tau40 fibrillization is complete under these conditions, as determined by ThT fluorescence and centrifugation assays. A non-assembly control reaction was also prepared as above with Tau40 and 5b in the absence of heparin. The completed reactions were centrifuged at 40,000 g for 30 minutes to sediment fibrils and the supernatants were applied to a Superdex 200 10/300 column (GE Healthcare) employing an Äkta Basic FPLC unit (GE Healthcare) with a flow rate of 0.4 ml/minute. Tau elution was monitored by absorbance at 280 nm. Under these chromatography conditions, monomeric tau can be separated from oligomeric species (Li, W. K. and Lee, V. M. Y. (2006) Biochemistry 45, 15692-15701).

### Compound Evaluation In Vitro.

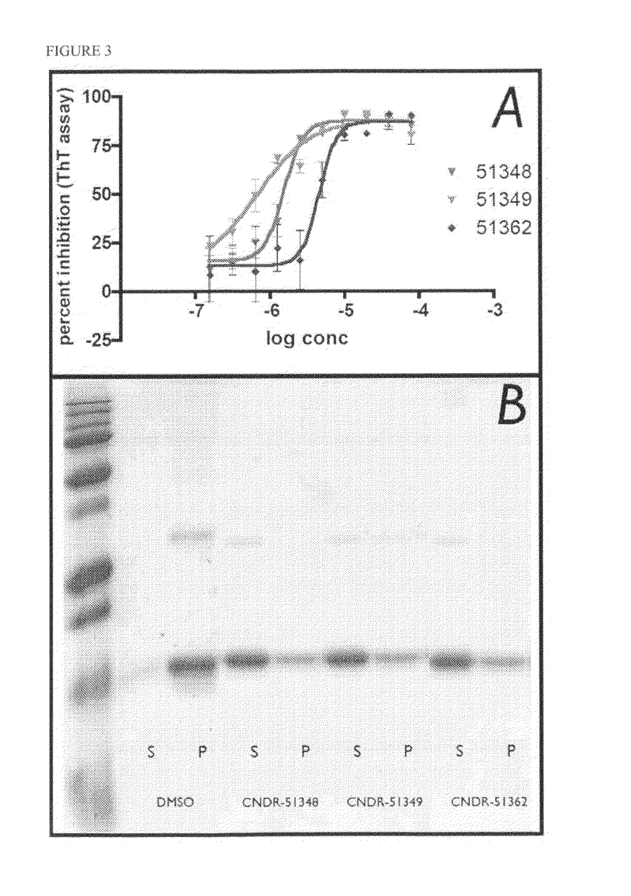

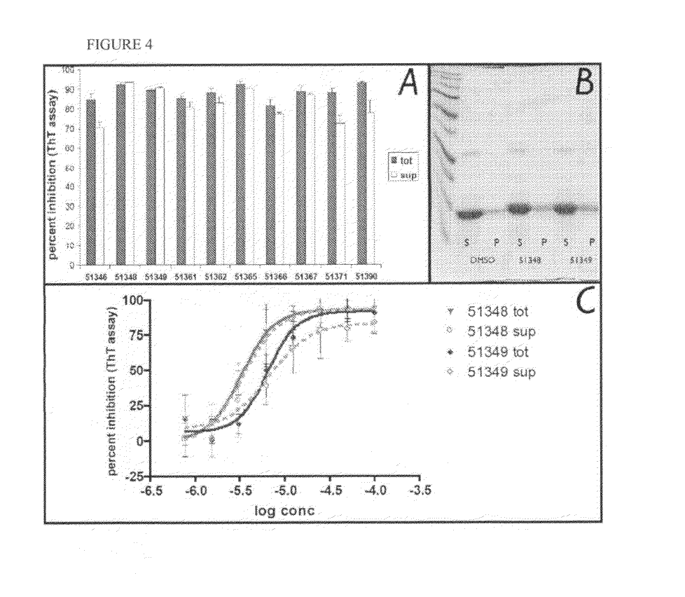

The solubility of test compounds in the sodium acetate buffer used for subsequent fibrillization reactions was determined by turbidimetric measurements. Compounds were then evaluated in a heparin-induced tau assembly assay, in which the fibrillization of the truncated K18 tau fragment (comprised of four MT-binding repeats) bearing the P301L mutation (K18PL) found in FTDP-17, was monitored by thioflavine-T (ThT) binding and fluorescence (FIG. 3A). (Crowe, A.; et al., Identification of aminothienopyridazine inhibitors of tau assembly by quantitative high-throughput screening. Biochemistry 2009, 48, 7732-45.) The inhibitory activity of test compounds was then confirmed with an orthogonal sedimentation assay in which compound-treated fibrillizing mixtures were centrifuged and quantitation of K18PL tau in the soluble and insoluble fraction was performed by densitometric analyses of SDS-PAGE (FIG. 3B). As summarized in Tables 3-5, all ATPZs exhibited IC₅₀ values in the 1-7 μM range in the primary ThT assay, with maximal percent inhibition of 70-90%. The activity against tau fibrillization was confirmed by sedimentation assay, where the majority of compounds exhibited >50% reduction in pelletable material compared to the untreated control. Since many of the ATPZ compounds were found to reach a solubility limit within the concentration range used in the biochemical assay, and considering that compound aggregation is known to be a potential mechanism of non specific inhibition (McGovern, S. L.; Helfand, B. T.; Feng, B.; Shoichet, B. K. A specific mechanism of nonspecific inhibition. J. Med. Chem. 2003, 46, 4265-72) additional experiments were conducted to ensure that the observed inhibitory activity of test compounds was not artifactual. Thus, the tau fibrillization assay was repeated in which both DMSO- and compound-treated mixtures were initially centrifuged for 30 min. prior to incubation (i.e., at time zero of the fibrillization reaction) to remove any pelletable material. As shown in FIG. 4A, no significant differences in compound-mediated inhibition of tau assembly were observed with and without the pre-centrifugation step. Furthermore, the IC₅₀ values of the centrifuged and control samples did not appear to differ appreciably (FIG. 4C). Moreover, SDS-PAGE analysis of the soluble and pelletable material after the initial centrifugation step revealed no significant difference between compound- and vehicle-treated mixtures; in all cases K18PL remained largely in the soluble fraction (FIG. 4B) and thus was not artifactually precipitated by the test compound. The activity and SDS-PAGE data suggest that the inhibition of K18PL fibrillization produced by the ATPZ analogs is not caused by insoluble or aggregated material. Furthermore, the absence of any correlation between compound solubility in buffer alone and activity in the tau fibrillization assay (Table 5) suggests that the solubilities of test compounds are somewhat increased in the presence of heparin and K18PL.

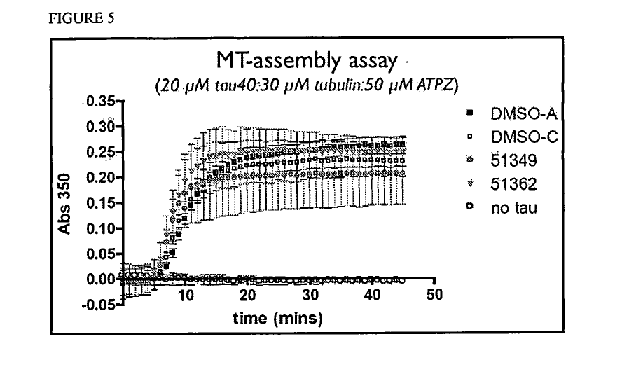

Notably, compounds also inhibited the aggregation of full-length tau (tau40) as determined by sedimentation assay, which revealed efficacy values in the 50-60% range (cf., Table 5). Compounds E1, 17, 2, and F1-F7 did not appear to interfere with the normal MT-stabilizing function of tau in a MT-polymerization assay (examples shown in FIG. 5). Finally, to evaluate possible major toxicities associated with the ATPZs, compounds were evaluated in a cytotoxicity assay that employs rapidly dividing HEK-293 cells. All tested compounds were found to be non-toxic at 100 μM concentration.

### Pharmacokinetic Studies.

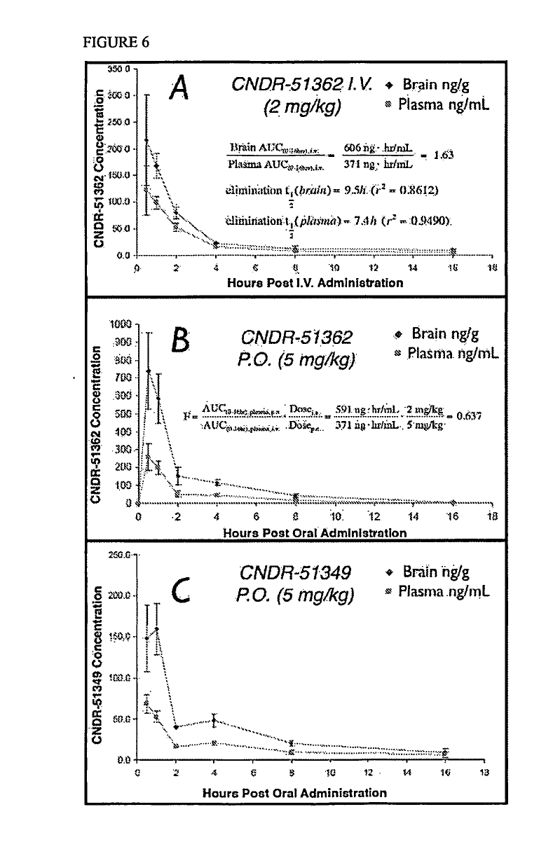

Test compounds underwent preliminary evaluations of brain penetration, in which 5 mg/Kg of each compound was administered intraperitoneally (i.p.) to a group of three normal mice; drug levels in brain and plasma were determined at a single time-point (1 h) by LC-MS/MS using pre-validated calibration curves. The results of these experiments, summarized in Table 5, revealed that with the exception of CNDR-51348 (E1), all other test compounds displayed significant brain uptake as demonstrated by the brain-to-plasma exposure ratios (B/P) above 0.3. The lack of brain penetration of CNDR-51348 was not unexpected given that the carboxylic acid moiety of this compound would be mostly negatively charged at physiological pH, and thus likely result in limited passive diffusion of the compound across the BBB. Conversely, the more lipophilic ester derivatives (CNDR-51346 and CNDR-51371) exhibited comparatively higher B/P ratio. In particular, CNDR-51371 (2) was found to reach significantly higher brain concentrations compared to the corresponding acid CNDR-51348. However, both esters appeared to have relatively short half-lives in plasma, as indicated by the limited amount of parent drug detected after 1 h from administration of the compounds. Moreover, monitoring for the hydrolyzed metabolite (i.e., CNDR-51348) in both brain and plasma revealed a considerable amount of acid CNDR-51348 in plasma, but not in the brain 1 h after administration of either ester. CNDR-51362 was selected for full PK analysis in which brain and plasma drug levels were determined at six time points (i.e., 30 min, 1 h, 2 h, 4 h, 8 h, and 16 h). The results from this experiment, illustrated in FIG. 6A, confirmed that the total B/P exposure ratio over 16 h is 1.6. Furthermore, these data reveal that CNDR-51362 exhibits good metabolic stability as indicated by an elimination half-life of ˜9.5 h in the brain and ˜7.4 h in plasma. Finally, in order to investigate the potential for ATPZ congeners to be orally administered, we evaluated brain and plasma level of CNDR-51362 and CNDR-51349 after oral administration of 5 mg/Kg. Notably, in both cases, significant concentrations of each test compound was achieved in the brain after oral administration (cf., FIGS. 6B and 6C). Comparison of the integrated area under the curve (AUC) in plasma after oral and i.v. administration of CNDR-51362 revealed an oral bioavailability of ˜64% (cf, FIGS. 6A and 6B).

### Solubility Studies.

Compounds were serially diluted in DMSO to achieve a 2.5 fold dilution series from 20 mM down 8 points to 3 μM on a 384-well master plate (Costar 3672). Aliquots (2 μL) were transferred to a clear 96-well assay plate (Fisher 12-565-501) containing 198 μL fibrillization buffer (100 mM NaOAc pH 7.0) for a 100-fold dilution (200 μM down to 0.3 μM). The plate was incubated with agitation for 2.5 h, and the absorbance at 550, 600, 650 and 700 nm was measured on a Spectramax M5 spectrophotometer. DMSO (1%) in fibrillization buffer served as a control that was subtracted from each data point. The absorbances were averaged and the concentration at which the average absorbance rose above 0.03 AU was an indication of insoluble compound, with solubility limits reported as the previous concentration in the dilution series. Absorbance scans of compounds in DMSO indicated that the compounds tested here did not have any absorbance peaks in the above range, therefore all absorbance was due to light scattering by insoluble material.

### Cytotoxicity Assay.

Cells were plated in each well of a 96-well plate in 100 μL of media (20,000 cells; DMEM plus 10% fetal calf serum with antibiotics). After 24 h, half-log serial dilutions of the test compounds, starting at 200 μM, were added in a further 100 μL of media. The cells were cultured for an additional 72 h and then Alamar blue (Biosource, Camarillo, Calif.) was added, with cell viability at each drug concentration measured using a SpectraMax M5 fluorescence plate-reader (Molecular Devices, Sunnyvale, Calif.). CC₅₀ values were generated using Prism software.

### Determination of Plasma and Brain Drug Concentrations.

Brain tissue was homogenized in 10 mM ammonium acetate, pH 5.7 (1:2; w/v) using a handheld sonic homogenizer. Plasma was obtained from blood that was collected into a 1.5 mL tube containing 0.5M EDTA solution and subjected to centrifugation for 10 min. at 4500 g at 4° C. Aliquots (50 μL) of brain homogenate or plasma were mixed with 0.2 mL of acetonitrile, centrifuged at 15,000 g, and the resulting supernatants were used for subsequent LC-MS/MS analysis. The LC-MS/MS system was comprised of an Aquity HPLC and a TQ MS that was controlled using MassLynx software (Waters Corporation, Milford, Mass., USA). Compounds were detected using multiple reaction monitoring (MRM) of their specific collision-induced ion transitions. Samples (5 μL) were separated on an Aquity BEH C18 column (1.7 μm, 2.1×50 mm) at 35° C. Operation was in positive electrospray ionization mode, with mobile phase A of 0.1% (v/v) formic acid, and B of either acetonitrile or methanol with 0.1% (v/v) formic acid at a flow rate of 0.6 mL/min using a gradient from 5% to 95% B over two min., followed by wash and re-equilibration steps. The MS was operated with a desolvation temperature of 450° C. and a source temperature of 150° C. Desolvation and source nitrogen gas flows were 900 L/hr and 50 L/hr, respectively. Source and MS/MS voltages were optimized for each compound using the MassLynx auto tune utility. Standard curves were generated for each compound from brain homogenate and plasma samples that had compound added at 4, 40, 400 and 4000 ng/mL and extracted as above. Peak areas were plotted against concentration and a 1/x weighted linear regression curve was used to quantify the tissue-derived samples using the average peak area from triplicate injections.

### Synthesis of ATPZ's

Compounds 16-19 were purchased from commercial sources. Compounds 5a-5h and 6a-b were synthesized (Scheme 1) following a modified literature procedure (Ferguson, G. N., Valant, C., Home, J., Figler, H., Flynn, B. L., Linden, J., Chalmers, D. K., Sexton, P. M., Christopoulos, A., and Scammells, P. J. (2008) Journal of Medicinal Chemistry 51, 6165-6172). Commercially available anilines 1a-h were converted to aryl diazonium salts 2, which reacted with β-ketoesters to form hydrazones 3 as a mixture of E/Z isomers. Condensation reactions of the hydrazones with ethyl cyanoacetate gave pyridazines 7. Subsequently reactions of pyridazines 7 with sulfur under Gewald conditions generated 5a-h. Saponification of 5a-b gave the corresponding 6a-b.

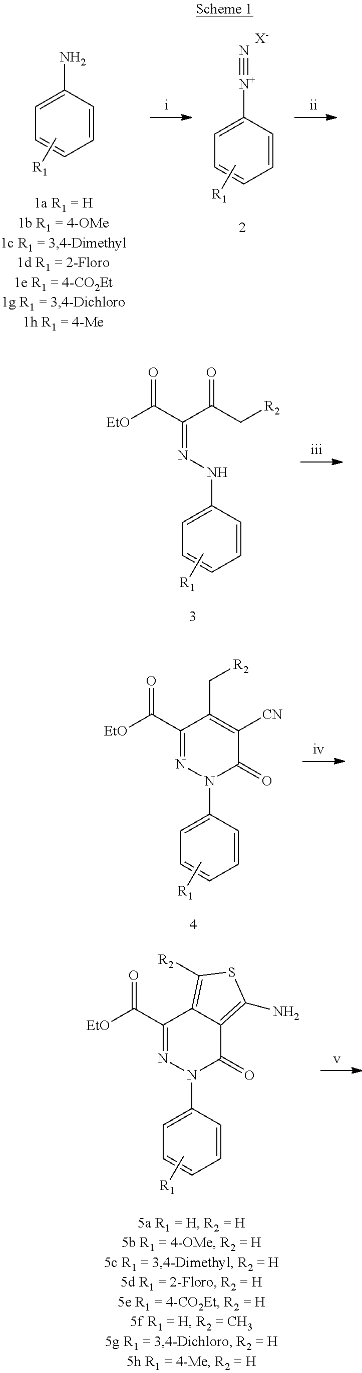

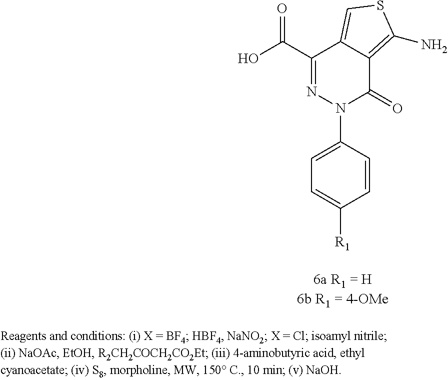

Compound 8d was synthesized by a DMC-mediated coupling reaction between acid 6b and diethyl amine. Molecules 8a-c were prepared by a reaction sequence starting from 7a as depicted in Scheme 2. Hydrolysis of 4a under acidic condition yielded 7a. Amide 7b was formed by reacting 4a with ammonium. Coupling reactions of acid 7a with appropriate amines followed by Gewald reaction yielded the desired amides 8a-c.

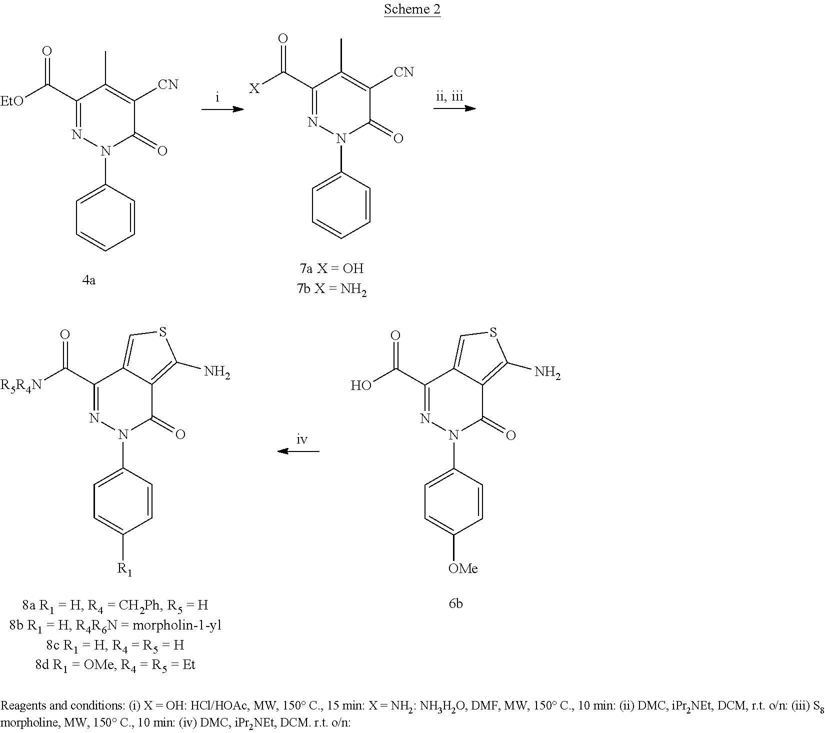

Compounds 9a-c were prepared by reactions of 5a-b with appropriate acid chlorides, Scheme 3.

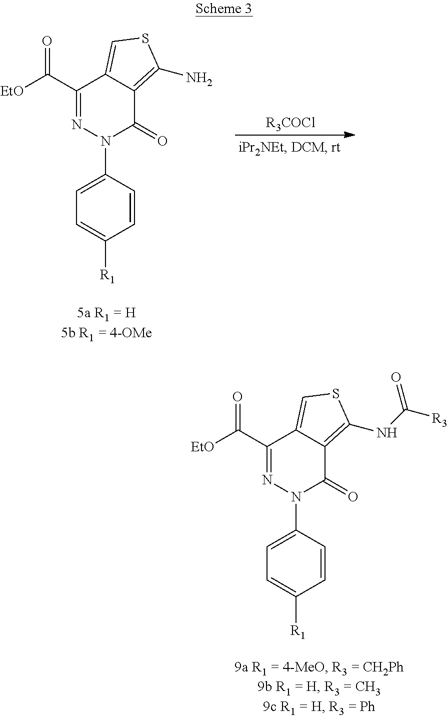

The preparation of 14 is illustrated in Scheme 4. EDC-mediated coupling reaction of acid 7a with tert-butylcarbazate followed by Boc-deprotection gave hydrazide 10. The hydrazide reacted with nitrous acid in acetic acid produced carboazide 11. Curtius rearrangement of 11 in ethanol gave ethyl carbamate 13. Under basic condition, the carbamate was converted to the desired amine 14. Compound 15 was prepared by LiAlH₄ reduction of ester 5a, Scheme 5.

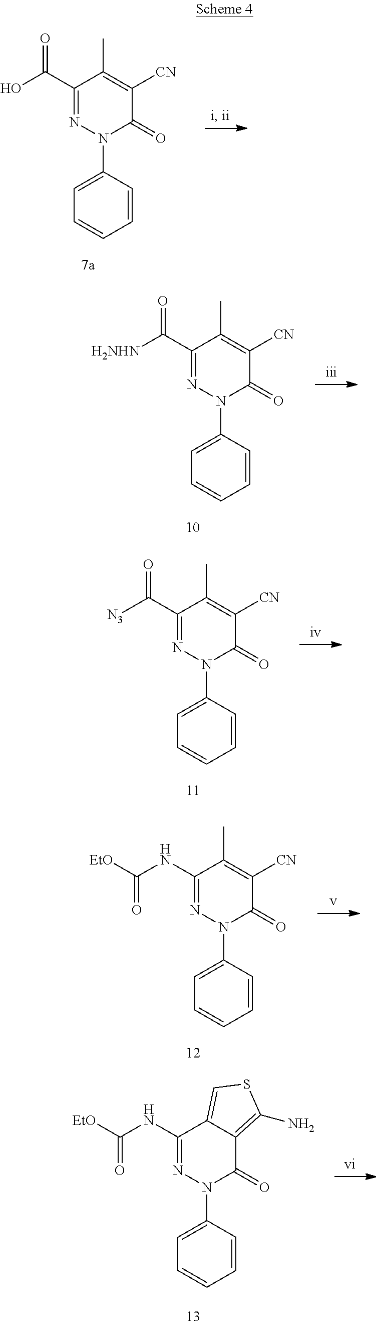

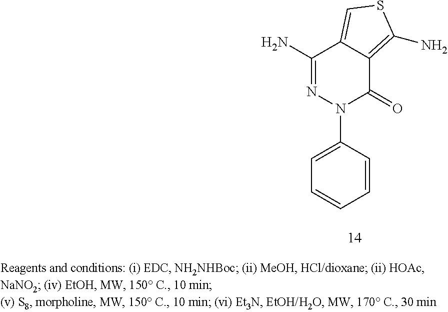 [SMILES: Nc1nn(-c2ccccc2)c(=O)c2c(N)scc12]

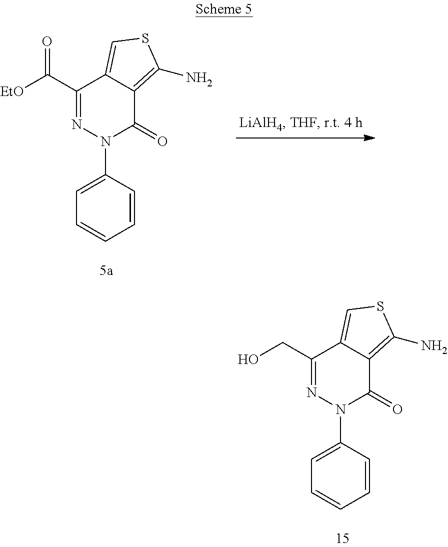 [SMILES: CCOC(=O)c1nn(-c2ccccc2)c(=O)c2c(N)scc12 ; Nc1scc2c(CO)nn(-c3ccccc3)c(=O)c12]

The synthesis of compounds D1-D24, E1-E6, and F1-15 was achieved via the reaction sequence illustrated in Scheme 6, which entails (a) a diazonium coupling reaction to form hydrazones of general structure B; (b) a Knovenagel-type condensation between B and ethyl cyanoacetate to form pyridazines C;^{23, 24} and (c) a Gewald aminothiophene synthesis²⁵ to furnish the desired ATPZs of general structure D. Compound 17 was then trans-esterified to the corresponding isopropyl ester, CNDR-51371 (2), upon treatment with Ti(iPrO)₄ in isopropanol. Alternatively, compounds of general structure D were saponified, and the resulting acids E employed in series of BOP coupling reactions with various primary and secondary amines to furnish compounds F1-7.

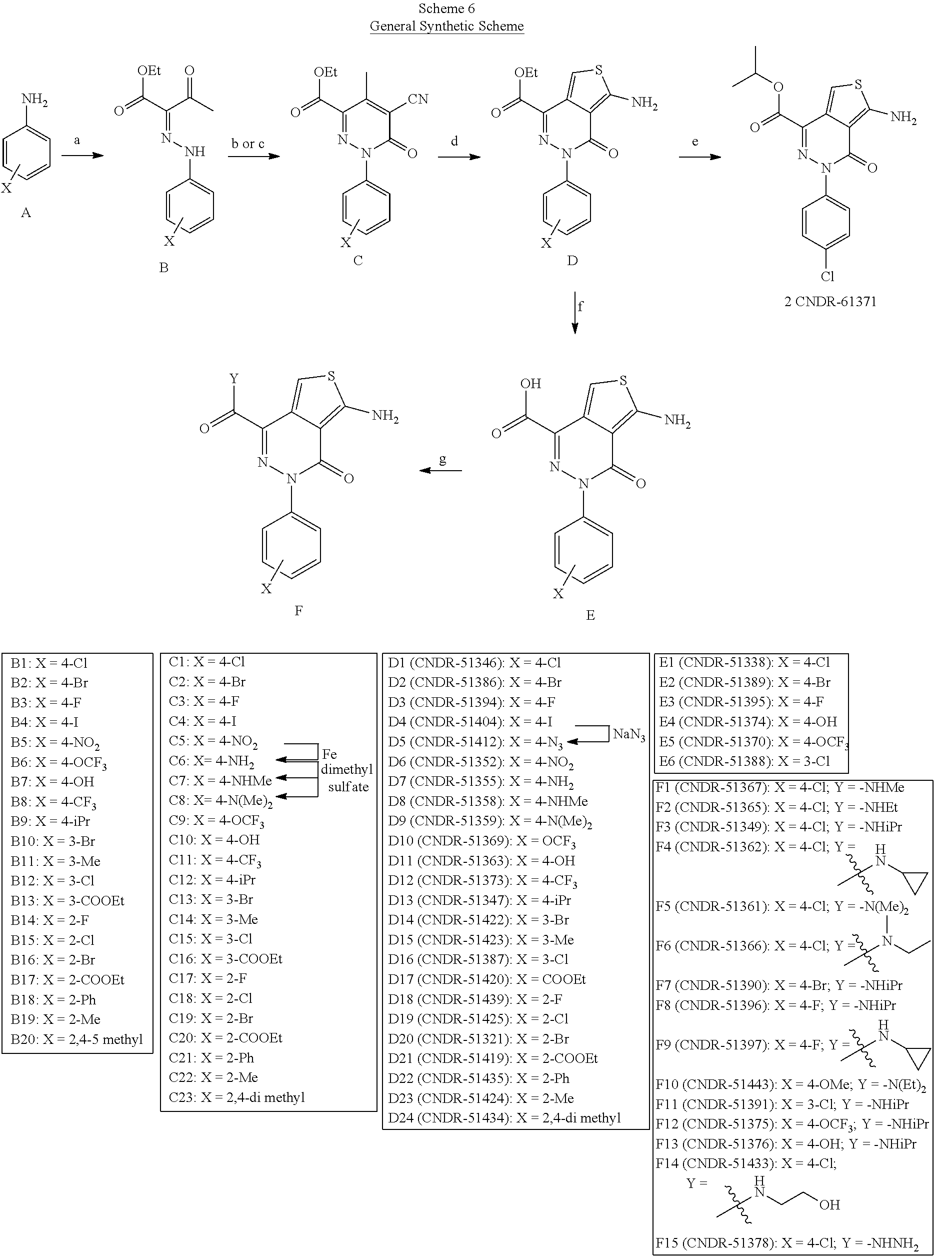

Scheme 6. Reagents and Reaction conditions: a) (i) NaNO₂, 37% HCl, ethanol, water, 0° C., 20 min; (ii) ethyl acetoacetate, sodium acetate, ethanol, water, 0° C., 2 h; b) ethyl cyanoacetate, ammonium acetate, acetic acid, 170° C. (microwave irradiation), 4 min; c) ethyl cyanoacetate, 4-aminobutyric acid, 160° C., 2.5 h; d) S₈, morpholine, ethanol, 150° C. (microwave irradiation), 15 min; e) LiOH.H₂O, tetrahydrofuran, water, rt, 16 h; f) appropriate amine, BOP reagent, N,N-diisopropylethylamine, dimethylsulfoxide, rt, 4 h.

The synthesis of CNDR-51360 was conducted by treating 17 with methyl iodide (Scheme 7).

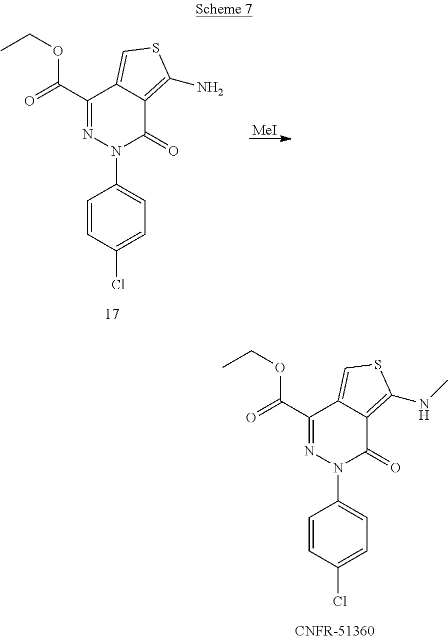 [SMILES: "MeI" ; CCOC(=O)c1nn(-c2ccc(Cl)cc2)c(=O)c2c(N)scc12 ; CCOC(=O)c1nn(-c2ccc(Cl)cc2)c(=O)c2c(NC)scc12 ; "CNFR-51360"]

The synthesis of CNDR-51411 and CNDR-51393 was conducted as depicted in Scheme 8, i.e., by treating 17 with either N-bromosuccinimide to form CNDR-51411, or with N-chlorosuccinimide to form CNDR-51393.

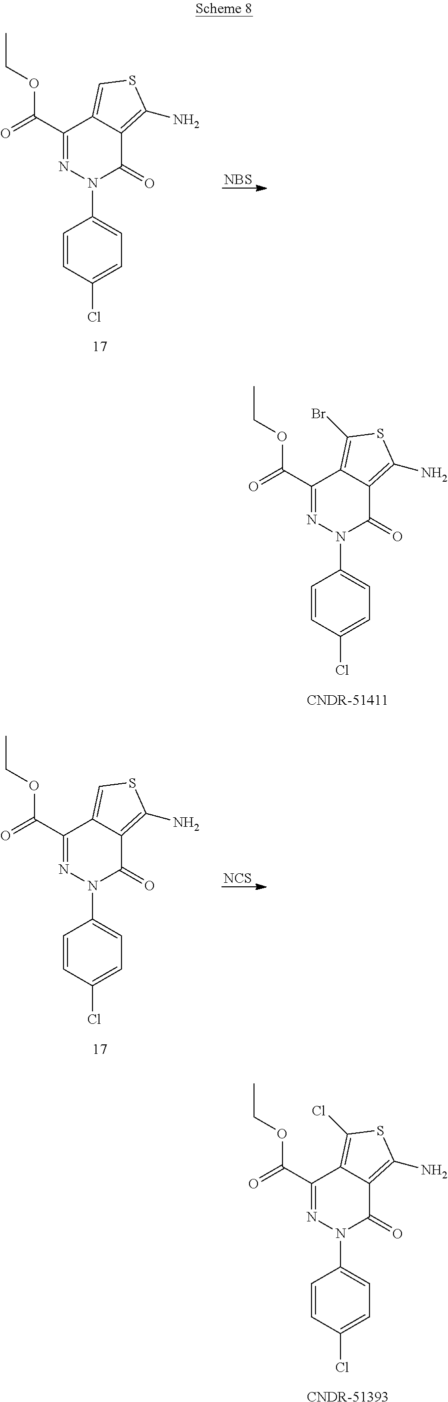

The synthesis of compounds of general structure h was conducted as depicted in Scheme 9, starting from compound 11.

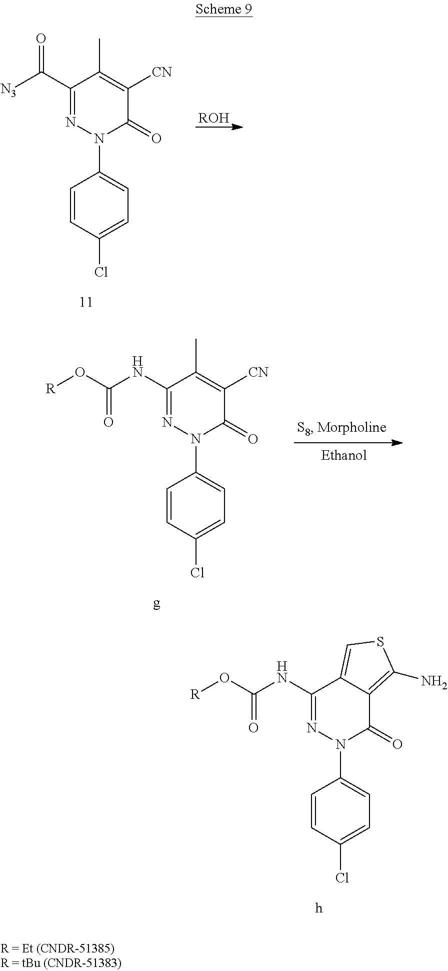

### Compound Identity and Purity Determination

All compounds underwent purity and mass determination using reverse-phase HPLC/MS (ESI⁺) method with elution monitoring by UV absorbance (220 nm) and evaporative light scatter (ELS). Analyses were performed at a flow rate of 0.5 ml/min on a Waters Acquity HPLC system using a Phenomenex 2.5 μm Luna C18(2)-HST 100×2 mm column at 45° C. A linear 2-100% acetonitrile (0.025% TFA)/H₂O (0.05% TFA) gradient was utilized over 2.2 min, with a total run time of 3.0 min. Purity was assessed by integration of chromatograms (UV_{220 nm} and ELSD).

### Materials and Methods

All solvents were reagent grade. All reagents were purchased from Aldrich or Acros and used as received. Thin layer chromatography (TLC) was performed with 0.25 mm E. Merck pre-coated silica gel plates. Flash chromatography was performed with silica gel 60 (particle size 0.040-0.062 mm) supplied by Silicycle and Sorbent Technologies. TLC spots were detected by viewing under a UV light. Infrared (IR) spectra were recorded on a Jasco Model FT/IR-480 Plus spectrometer. All melting points were obtained on a Thomas-Hoover apparatus. Proton (¹H) and carbon (¹³C) NMR spectra were recorded on a Bruker AMX-500 spectrometer. Chemical shifts were reported relative to solvents. High-resolution mass spectra were measured at the University of Pennsylvania Mass Spectrometry Center on either a VG Micromass 70/70H or VG ZAB-E spectrometer. Single-crystal X-ray structure determinations were performed at the University of Pennsylvania with an Enraf Nonius CAD-4 automated diffractometer. Analytical reverse-phased (Sunfire™ C18; 4.6×50 mm, 5 mL) high-performance liquid chromatography (HPLC) was performed with a Waters binary gradient module 2525 equipped with Waters 2996 PDA and Waters micromass ZQ. All samples were analyzed employing a linear gradient from 10% to 90% of acetonitrile in water over 8 minutes and flow rate of 1 mL/min, and unless otherwise stated, the purity level was >95%. Preparative reverse phase HPLC purifications were performed on a Gilson instrument (i.e., Gilson 333 pumps, a 215 liquid handler, 845Z injection module, and PDA detector) employing Waters SunFire™ preparative C₁₈ OBD™ columns (5 μm 19×50 or 19×100 mm). Purifications were carried out employing a linear gradient from 10% to 90% of acetonitrile in water for 15 minutes with a flow rate of 20 mL/min. Yields refer to chromatographically and spectroscopically pure compounds.

### Ethyl 2-(2-(4-chlorophenyl)hydrazono)-3-oxobutanoate (B1)

Prepared as previously reported. (Nagakura, M. et al., J. Med. Chem, 1979, 22, 48-52) Yield: 83%, mp: 84-85° C. (from ethanol, Lit. 81-84° C. Id.). ¹H NMR (CDCl₃): δ 1.41 (t, J=7.0 Hz, 3H), 2.60 (s, 3H), 4.34 (q, J=7.2 Hz, 2H), 7.34-7.36 ppm (m, 4H). ¹³C NMR (CDCl₃): δ 14.5, 30.9, 61.2, 117.6, 126.5, 129.8, 131.0, 140.4, 164.9, 197.4 ppm. IR: v 3359, 1706, 1617 cm⁻¹. HRMS (ESI⁺): calculated for C₁₂H₁₃ClN₂NaO₃⁺ 291.0518 found 291.0512.

### Ethyl 2-(2-(4-bromophenyl)hydrazono)-3-oxobutanoate (B2)

4-Bromoaniline (2.0 g, 11.63 mmol) was dissolved in 37% hydrochloridic acid (2.94 mL), ethanol (1.50 mL) and water (1.50 mL). The reaction mixture was cooled to 0° C. in an ice water bath before a solution of sodium nitrite (0.88 g, 12.75 mmol) in water (2.0 mL) was added dropwise. The resulting mixture was stirred at 0° C. for 20 min. Sodium acetate (3.72 g, 45.3 mmol) in water (5.9 mL) and ethyl acetoacetate (1.51 g, 1.43 mL, 11.63 mmol) were added and the reaction mixture was stirred at 0° C. for 2 h. The precipitated solid was then filtered, washed with water, and dried under high vacuum for 16 h to provide B2 as yellow solid that was crystallized in ethanol and used without further purification in the next step. Yield: 98%.

### Ethyl 1-(4-chlorophenyl)-5-cyano-4-methyl-6-oxo-1,6-dihydropyridazine-3-carboxylate (C1)

Prepared as previously reported. (Sotelo, E. et al., Synth Commun. 1997, 27, 2419-23) Yield: 47%, mp: 160-161° C. (from ethanol, Lit. 190° C. Id.). NMR (CDCl₃): δ 1.41 (t, J=7.0 Hz, 3H), 2.76 (s, 3H), 4.43 (q, J=7.2 Hz, 2H), 7.48 (d, J=8.5 Hz, 2H). 7.62 ppm (d, J=8.5 Hz, 2H). ¹³C NMR (CDCl₃): δ 14.2, 19.4, 63.0, 112.4, 116.0, 126.3, 129.4, 135.5, 137.5, 138.5, 150.8, 155.8, 162.0 ppm. IR: v 2235, 1726, 1685 cm⁻¹. MS [ESI⁺]: calculated for C₁₅H₁₃ClN₃O₃⁺ 318.06 found 318.07.

### Ethyl 1-(4-bromophenyl)-5-cyano-4-methyl-6-oxo-1,6-dihydropyridazine-3-carboxylate (C2)

A mixture of B2 (2.0 g, 6.39 mmol), 4-aminobutiryc acid (1.33 g, 12.99 mmol), and ethyl cyanoacetate (1.1 g, 1.0 mL, 9.44 mmol) was stirred neat at 160° C. for 2.5 h. After cooling the residue was purified by silica gel flash chromatography (using an ethyl acetate/n-hexane gradient of 5% to 80% of ethyl acetate) to provide C2 as white solid. Yield: 76%, mp: 174-176° C. (from ethanol). NMR (CDCl₃): δ 1.41 (t, J=7.0 Hz, 3H), 2.76 (s, 3H), 4.43 (q, J=4.43 Hz, 7.2 Hz, 2H), 7.56 (d, J=9.0 Hz, 2H), 7.64 ppm (d, J=9.0 Hz, 2H). ¹³C NMR (CDCl₃): δ 14.2, 19.4, 62.9, 112.4, 116.0, 123.5, 126.6, 132.3, 137.6, 139.1, 150.8, 155.7, 162.0 ppm. IR: v 2230, 1727, 1681 cm⁻¹.

### Synthesis of Thienyl Derivatives with General Structure D (Gewald Reaction)

### Representative Example Ethyl 5-amino-3-(4-chlorophenyl)-4-oxo-3,4-dihydrothieno[3,4-d]pyridazine-1-carboxylate (17)

A mixture of C1 (0.100 g, 0.31 mmol), sulfur (0.015 g, 0.47 mmol), and morpholine (0.55 g, 0.55 mL, 0.63 mmol) was heated to 150° C. using microwave irradiation for 15 min. After cooling, the precipitate which formed was collected and purified by preparative HPLC to give 17 as yellow solid. HPLC-MS retention time 8.20 min. Yield: 64%, mp: 189-191° C. (from ethanol). ¹H NMR (CDCl₃): δ 1.43 (t, J=7.3 Hz, 3H), 4.44 (q, J=7.2 Hz, 2H), 6.21 (broad s, 2H), 7.25 (s, 1H), 7.42 (d, J=8.5 Hz, 2H), 7.56 ppm (d, J=8.5 Hz, 2H). ¹³C NMR (CDCl₃): δ 14.4, 62.2, 104.9, 106.9, 127.1, 127.2, 128.9, 133.3, 134.0, 139.2, 159.4, 161.8, 163.0 ppm. IR: v 3411, 3303, 1724 cm⁻¹. HRMS (ESI⁺): calculated for C₁₅H₁₃ClN₃O₃S⁺ 350.0366 found 350.0355.

### Ethyl 5-amino-3-(4-bromophenyl)-4-oxo-3,4-dihydrothieno[3,4-d]pyridazine-1-carboxylate (D2)

Was synthesized in the same manner as 17 starting with C2. Yield: 55%, mp: 198-200° C. (from ethanol). ¹H NMR (CDCl₃): δ 1.42 (t, J=7.0 Hz, 3H), 4.44 (q, J=7.2 Hz, 2H), 6.12 (broad s, 2H), 7.25 (s, 1H), 7.51 (d, J=8.5 Hz, 2H), 7.58 ppm (d, J=8.5 Hz, 2H). ¹³C NMR (CDCl₃): δ 14.1, 62.1, 105.9, 106.7, 121.3, 127.1, 127.5, 131.8, 134.0, 139.7, 159.4, 161.9, 163.0 ppm. IR: v 3407, 3310, 1709, 1657 cm⁻¹. HRMS (ESI⁺): calculated for C₁₅H₁₃BrN₃O₃S⁺ 393.9861 found 393.9879.

### Synthesis of Compounds with General Structure E (Esters Hydrolysis)

### Representative Example: 5-Amino-3-(4-chlorophenyl)-4-oxo-3,4-dihydrothieno[3,4-d]pyridazine-1-carboxylic acid (E1)

Lithium hydroxide monohydrate (0.063 g, 1.29 mmol) was added to a solution of 17 (0.150 g, 0.43 mmol) in tetrahydrofuran (3 mL) and water (2 mL). The reaction mixture was stirred at room temperature for 16 h. 1 N HCl was added (pH ˜2), the formed precipitate was filtered and purified by preparative HPLC to afford E1 as yellow solid. HPLC-MS retention time 6.63 min. Yield: 67%, mp: >300° C. (from ethanol). ¹H NMR (CD₃OD): δ 7.19 (s, 1H), 7.44 (d, J=9.0 Hz, 2H), 7.58 ppm (d, J=9.0 Hz, 2H). ¹³C NMR (DMSO-d₆): δ 104.4, 104.5, 126.8, 128.3, 128.9, 132.0, 134.5, 140.2, 158.9, 163.9, 164.6 ppm. IR: v 3439, 3322, 3065, 1712, 1646, 1590 cm⁻¹. HRMS (ESP): calculated for C₁₃H₉ClN₃O₃S⁺ 322.0053 found 322.0062.

### 5-Amino-3-(4-bromophenyl)-4-oxo-3,4-dihydrothieno[3,4-d]pyridazine-1-carboxylic acid (E2)

Prepared in the same manner as E1 starting with D2. HPLC-MS retention time 6.76 min. Yield: 77%, mp: >300° C. (from ethanol). ¹H NMR (DMSO-d₆): δ 7.12 (s, 1H), 7.52 (d, J=9.0 Hz, 2H), 7.63 (broad s, 2H), 7.66 ppm (d, J=8.5 HZ, 2H). ¹³C NMR (DMSO-d₆): δ 104.4, 104.5, 120.4, 126.8, 128.6, 131.9, 134.5, 140.6, 158.8, 163.9, 164.5 ppm. IR: v 3439, 3322, 3065, 1712, 1646 cm⁻¹. HRMS (ESI⁴): calculated for C₁₃H₇BrN₃NaO₃S⁺ 387.9367 found 387.9378.

### Isopropyl 5-amino-3-(4-chlorophenyl)-4-oxo-3,4-dihydrothieno[3,4-d]pyridazine-1-carboxylate (2)

A mixture of 17 (0.050 g, 0.143 mmol) and titanium (IV) isopropoxide (0.002 g, 2.1 μL, 7 μmol) in isopropanol (0.5 mL) was heated to 170° C. using microwave irradiation for 40 min. After cooling, the solvent was evaporated and the residue was purified by preparative HPLC to provide 2 as yellow solid. HPLC-MS retention time 8.73 min. Yield: 77%, mp: 118-120° C. (from ethanol). ¹H NMR (CDCl₃): δ 1.41 (d, J=6.5 Hz, 6H), 5.24-5.33 (m, 1H), 6.25 (broad s, 2H), 7.17 (s, 1H), 7.40 (d, J=8.5 Hz, 2H), 7.57 ppm (d, J=8.5 Hz, 2H). ¹³C NMR (CDCl₃): δ 22.0, 70.1, 104.7, 106.8, 127.1, 127.2, 128.8, 133.1, 134.4, 139.2, 159.4, 161.9, 162.5 ppm. IR: v 3408, 3294, 3145, 1716, 1663, 1585 cm⁻¹. HRMS (ESI⁺): calculated for C₁₆H₁₄ClN₃O₃NaS⁺ 386.0342 found 386.0331.

### Synthesis of Compounds with General Structure F

### Representative Example: 5-Amino-3-(4-chlorophenyl)-N-isopropyl-4-oxo-3,4-dihydrothieno[3,4-d]pyridazine-1-carboxamide (F3)

DIPEA (0.012 g, 14 μL, 0.093 mmol) was added to a mixture of E1 (0.020 g, 0.062 mmol), isopropylamine (0.007 g, 11 μL, 0.124 mmol), and BOP reagent (0.041 g, 0.093 mmol) in anhydrous DMSO (1 mL). The reaction mixture was stirred at room temperature for 4 h. Water was added and the resulting mixture was extracted with ethyl acetate. The organic layer was washed with brine, dried over MgSO₄, filtered and evaporated. The residue was purified by preparative HPLC to give the F3 as yellow solid. HPLC-MS retention time: 8.04 min. Yield: 89%, mp: 197-199° C. (from ethanol). ¹H NMR (CD₃OD): δ 1.22 (d, J=6.5 Hz, 6H), 4.09-4.19 (m, 1H), 7.25 (s, 1H), 7.46 (d, J=8.5 Hz, 2H), 7.59 ppm (d, J=8.5 Hz, 2H). ¹³C NMR (CD₃OD): δ 19.6, 39.9, 102.9, 103.3, 124.9, 126.1, 126.9, 131.2, 135.1, 138.1, 158.0, 161.4, 162.1 ppm. IR: v 3404, 3294, 3178, 1661 cm⁻¹. HRMS (ESI⁺): calculated for C₁₆H₁₅ClN₄O₂NaS⁺ 385.0502 found 385.0492.

### 5-Amino-3-(4-chlorophenyl)-N-methyl-4-oxo-3,4-dihydrothieno[3,4-d]pyridazine-1-carboxamide (F1)

Prepared in the same manner as F3 starting with E1 using methylamine (2.0 M solution in tetrahydrofuran). HPLC-MS retention time: 6.83 min. Yield: 52%, mp: 192-194° C. (from ethanol). ¹H NMR (DMSO-d₆): δ 2.75 (d, J=5.0 Hz, 3H), 7.24 (s, 1H), 7.51 (d, J=9.0 Hz, 2H), 7.60 (broad s, 2H), 7.69 (d, J=9 Hz, 2H), 8.27 ppm (broad s, 1H). ¹³C NMR (DMSO-d₆): δ 26.4, 104.5, 105.2, 126.6, 128.1, 128.8, 131.6, 136.8, 140.1, 158.9, 163.5, 163.6 ppm. IR: v 3418, 3302, 1656, 1593 cm⁻¹. HRMS (ESI⁺): calculated for C₁₄H₁₃ClN₄O₂S⁺ 333.0213 found 333.0210.

### 5-Amino-3-(4-chlorophenyl)-N-ethyl-4-oxo-3,4-dihydrothieno[3,4-d]pyridazine-1-carboxamide (F2)

Prepared in the same manner as F3 starting with E1 using ethylamine hydrochloride. HPLC-MS retention time: 7.43 min. Yield: 58%, mp: 203-205° C. (from ethanol). ¹H NMR (CDCl₃): δ 1.23 (t, J=7.3 Hz, 3H), 341-347 (m, 2H), 6.18 (broad s, 2H), 7.07 (s, 1H), 7.45 (d, J=9 Hz, 2H), 7.51 (d, J=9 Hz, 2H), 7.60 ppm (broad s, 1H). ¹³C NMR (CDCl₃): δ 14.9, 34.3, 106.8, 106.9, 126.7, 127.3, 129.0, 133.3, 135.6, 139.2, 159.5, 161.4, 162.6 ppm. IR: v 3405, 3297, 1652, 1600 cm⁻¹. HRMS (ESI⁺): calculated for C₁₅H₁₃ClN₄NaO₂S⁺ 371.0345 found 371.0341.

### 5-Amino-3-(4-chlorophenyl)-N-cyclopropyl-4-oxo-3,4-dihydrothieno[3,4-d]pyridazine-1-carboxamide (F4)

Prepared in the same manner as F3 starting with E1 using cyclopropylamine. HPLC-MS retention time: 7.43 min. Yield: 41%, mp: 188-190° C. (from ethanol). ¹H NMR (CD₃OD): δ 0.58-0.60 (m, 2H), 0.66-0.69 (m, 2H), 2.76-2.81 (m, 1H), 7.19 (s, 1H), 7.50, (d, J=9.0 Hz, 2H), 7.67 (d, J=9.0 Hz, 2H), 8.28 ppm (broad s, 1H). ¹³C NMR (CD₃OD): δ 6.3, 23.2, 104.4, 105.1, 126.6, 128.2, 128.7, 131.6, 137.0, 140.1, 158.9, 163.3, 164.4 ppm. IR: v 3408, 3302, 1654, 1593 cm⁻¹. HRMS (ESI⁺): calculated for C₁₆H₁₃Cl₄O₂N₄NaS⁺ 383.0345 found 383.0333.

### 5-Amino-3-(4-chlorophenyl)-N,N-dimethyl-4-oxo-3,4-dihydrothieno[3,4-d]pyridazine-1-carboxamide (F5)

Prepared in the same manner as F3 starting with E1 using dimethylamine hydrochloride. HPLC-MS retention time: 6.43 min. Yield: 62%, mp: 242-244° C. (from ethanol). ¹H NMR (CDCl₃): δ 3.14 (s, 3H), 3.16 (s, 3H), 6.18 (broad s, 2H), 6.73 (s, 1H), 7.41 (d, J=9.0 Hz, 2H), 7.52 ppm (d, J=9.0 Hz, 2H). ¹³C NMR (CD₃OD): δ 35.7, 39.0, 104.0, 106.9, 127.0, 128.1, 128.8, 132.9, 139.2, 139.3, 159.1, 161.7, 164.3 ppm. IR: v 3414, 3300, 1646, 1598 cm⁻¹. HRMS (ESI⁺): calculated for C₁₄H₁₃ClN₄NaO₂S⁺ 371.0345 found 371.0366.

### 5-Amino-3-(4-chlorophenyl)-N-ethyl-N-methyl-4-oxo-3,4-dihydrothieno[3,4-d]pyridazine-1-carboxamide (F6)

Prepared in the same manner as F3 starting with E1 using ethylmethylamine. HPLC-MS retention time 6.85 min. Yield: 47%, mp: 148-150° C. (from ethanol). ¹H NMR (CD₃OD): δ 1.19-1.26 (m, 3H), 3.48-3.60 (m, 2H), 6.16 (broad s, 2H), 6.70 (s, 1H), 7.40-7.42 (m, 2H), 7.51-7.54 ppm (m, 2H). ¹³C NMR (CD₃OD): δ 12.2, 14.0, 32.9, 36.3, 42.9, 46.1, 103.5, 106.4, 111.3, 117.0, 126.7, 126.8, 127.0, 127.1, 127.8, 127.9, 128.8 (×2), 132.8, 132.9, 159.1 (×2), 162.2, 164.0, 164.5 ppm. IR: v 3409, 3290, 3175, 1635 cm⁻¹. HRMS (ESI: calculated for C₁₆H₁₅ClN₄NaO₂S⁺ 385.0502 found 385.0491.

### 5-Amino-3-(4-bromophenyl)-N-isopropyl-4-oxo-3,4-dihydrothieno[3,4-d]pyridazine-1-carboxamide (F7)

Prepared in the same manner as F3 starting with E2. HPLC-MS retention time 8.20 min. Yield: 68%, mp: 222° C. dec (from ethanol). ¹H NMR (CD₃OD): δ 1.26 (d, J=5.0 Hz, 6H), 4.13-4.22 (m, 1H), 7.29 (s, 1H), 7.56 (d, J=8.5 Hz, 2H), 7.64 ppm (d, J=8.5 Hz, 2H). ¹³C NMR (CD₃OD): δ 19.6, 58.6, 103.0, 103.3, 119.0, 124.9, 126.3, 129.9, 135.1, 138.6, 157.9, 161.4, 162.1 ppm. IR: v 3412, 3305, 1655, 1598 cm⁻¹. HRMS (ESI⁺): calculated for C₁₆H₁₅BrN₄NaO₂S⁺ 428.9997 found 428.9986.

### Representative Examples of Hydrazones of General Structure B

### Ethyl 2-(2-(4-chlorophenyl)hydrazono)-3-oxobutanoate (B1)

Prepared as previously reported (Nagakura, M. et al., J. Med. Chem, 1979, 22, 48-52). Yield: 83%, mp: 84-85° C. (from ethanol, Lit. [Nagakura, M. et al., J. Med. Chem, 1979, 22, 48-52] 81-84° C.). NMR (CDCl₃): δ 1.41 (t, J=7.0 Hz, 3H), 2.60 (s, 3H), 4.34 (q, J=7.2 Hz, 2H), 7.34-7.36 (m, 4H) ppm. ¹³C NMR (CDCl₃): δ 14.5, 30.9, 61.2, 117.6, 126.5, 129.8, 131.0, 140.4, 164.9, 197.4 ppm. IR: v 3359, 1706, 1617 cm⁻¹. HRMS (ESI⁺): calculated for C₁₂H₁₃ClN₂NaO₃⁺ 291.0518 found 291.0512.

### Ethyl 2-(2-(4-bromophenyl)hydrazono)-3-oxobutanoate (B2)

4-Bromoaniline (2.0 g, 11.63 mmol) was dissolved in 37% hydrochloridic acid (2.94 mL), ethanol (1.50 mL) and water (1.50 mL). The reaction mixture was cooled to 0° C. in an ice water bath before a solution of sodium nitrite (0.88 g, 12.75 mmol) in water (2.0 mL) was added dropwise. The resulting mixture was stirred at 0° C. for 20 min. Sodium acetate (3.72 g, 45.3 mmol) in water (5.9 mL) and ethyl acetoacetate (1.51 g, 1.43 mL, 11.63 mmol) were added and the reaction mixture was stirred at 0° C. for 2 h. The precipitated solid was then filtered, washed with water, and dried under high vacuum for 16 h to provide B2 as yellow solid that was crystallized in ethanol and used without further purification in the next step. Yield: 98%.

### Ethyl 2-(2-(4-nitrophenyl)hydrazono)-3-oxobutanoate (B5)

Prepared as B1 starting from 4-nitroaniline. Yield: 90%; mp: 130-132° C. (from ethanol). LC-MS retention time: 7.52 min. ¹H NMR (CDCl₃): δ 1.41 (t, J=7.0 Hz, 3H), 2.53 (s. 3H), 4.40 (q, J=7.0 Hz, 2H), 7.41 (d, J=9.0 Hz, 2H), 8.28 (d. J=9.0 Hz, 2H) ppm. ¹³C NMR (CDCl₃): δ 14.2, 27.1, 62.3, 115.2, 116.1, 126.0, 130.5, 144.1, 146.8, 163.3, 194.2 ppm.

### Ethyl 2-(2-(4-isopropylphenyl)hydrazono)-3-oxobutanoate (B9)

Was synthesized as compound B1 from 4-i-propylaniline. Yield: 99%; yellow oil. ¹H NMR (CDCl₃): δ 1.28 (d, J=7.0 Hz, 6H), 1.40 (t, J=7.0 Hz, 3H), 2.76 (s, 3H), 2.98 (sept, J=6.8 Hz, 1H), 4.42 (q, J=7.1 Hz, 2H), 7.35 (d, J=8.5 Hz, 2H), 7.54 (d, J=8.5 Hz, 2H) ppm. ¹³C NMR (CDCl₃): δ 14.3, 19.3, 24.0, 34.1, 62.8, 112.6, 115.8, 124.9, 127.2, 137.1, 137.9, 150.5, 150.6, 156.0, 162.2 ppm.

### Ethyl 2-(2-(2,4-dimethylphenyl)hydrazono)-3-oxobutanoate (B20)

Prepared as B1 starting from 2,4-dimethylaniline. Yield: 94%, mp: 106-108° C. ¹H NMR (CDCl₃): δ 1.41 (t, J=7.1 Hz, 3H), 2.32 (s, 3H), 2.35 (s, 3H), 2.61 (s, 3H), 4.34 (q, J=7.1 Hz, 2H), 7.00 (s, 1H), 7.08 (d, J=8.3 Hz, 1H), 7.68 (d, J=7.8 Hz, 1H) ppm. ¹³C NMR (CDCl₃): δ 14.6, 17.1, 21.1, 30.8, 61.0, 115.8, 125.6, 126.3, 128.3, 131.6, 135.7, 137.8, 165.4, 196.9 ppm. IR: n 3319, 1698, 1616 cm⁻¹. MS [ESI]⁺: calculated for C₁₄H₁₉N₂O₃⁺ 263.14 found 263.15.

### Representative Examples of Pyridazines of General Structure C

### Ethyl 1-(4-chlorophenyl)-5-cyano-4-methyl-6-oxo-1,6-dihydropyridazine-3-carboxylate (C1)

Prepared as previously reported. (Sotelo, E. et al., Synth Commun. 1997, 27, 2419-23) Yield: 47%, mp: 160-161° C. (from ethanol, Lit. Sotelo, E. et al., Synth Commun. 1997, 27, 2419-23] 190° C.). ¹H NMR (CDCl₃): δ 1.41 (t, J=7.0 Hz, 3H), 2.76 (s, 3H), 4.43 (q, J=7.2 Hz, 2H), 7.48 (d, J=8.5 Hz, 2H). 7.62 (d, J=8.5 Hz, 2H) ppm. ¹³C NMR (CDCl₃): δ 14.2, 19.4, 63.0, 112.4, 116.0, 126.3, 129.4, 135.5, 137.5, 138.5, 150.8, 155.8, 162.0 ppm. IR: v 2235, 1726, 1685 cm⁻¹. MS [ESI⁺]: calculated for C₁₅H₁₃ClN₃O₃⁺ 318.06 found 318.07.

### Ethyl 1-(4-bromophenyl)-5-cyano-4-methyl-6-oxo-1,6-dihydropyridazine-3-carboxylate (C2)

A mixture of B2 (2.0 g, 6.39 mmol), 4-aminobutiryc acid (1.33 g, 12.99 mmol), and ethyl cyanoacetate (1.1 g, 1.0 mL, 9.44 mmol) was stirred neat at 160° C. for 2.5 h. After cooling the residue was purified by silica gel flash chromatography (using an ethyl acetate/n-hexane gradient of 5% to 80% of ethyl acetate) to provide C2 as white solid. Yield: 76%, mp: 174-176° C. (from ethanol). NMR (CDCl₃): δ 1.41 (t, J=7.0 Hz, 3H), 2.76 (s, 3H), 4.43 (q, J=4.43 Hz, 7.2 Hz, 2H), 7.56 (d, J=9.0 Hz, 2H), 7.64 (d, J=9.0 Hz, 2H) ppm. ¹³C NMR (CDCl₃): δ 14.2, 19.4, 62.9, 112.4, 116.0, 123.5, 126.6, 132.3, 137.6, 139.1, 150.8, 155.7, 162.0 ppm. IR: v 2230, 1727, 1681 cm⁻¹.

### Ethyl 1-(4-nitrophenyl)-5-cyano-4-methyl-6-oxo-1,6-dihydropyridazine-3-carboxylate (C5)

Was synthesized as C1 from B5. Yield: 53%; LC-MS retention time: 7.38 min. ¹H NMR (CDCl₃): δ 1.42 (t, J=7.0 Hz, 3H), 2.79 (s, 3H), 4.45 (d, J=7.1 Hz, 2H), 7.95 (d, J=8.5 Hz, 2H), 8.37 (d, J=8.5 Hz, 2H) ppm. ¹³C NMR (CDCl₃): δ 14.2, 19.5, 63.2, 112.1, 116.5, 124.6, 125.8, 138.3, 144.7, 147.6, 151.3, 155.6, 161.7 ppm. MS [ESI]⁺: calculated for C₁₅H₁₃N₄O₅⁺ 329.09 found 329.12.

### Ethyl 1-(4-aminophenyl)-5-cyano-4-methyl-6-oxo-1,6-dihydropyridazine-3-carboxylate (C6)

A solution of the C5 (0.25 g, 0.76 mmol) in acetic acid (5 mL) was heated to 60° C. and iron (0.21 g, 3.8 mmol) was added in small portion. The reaction mixture was stirred at 60° C. for 3 h. After cooling the solvent was evaporated and the residue was dissolved in water and ethyl acetate. The phases were separated and the aqueous layer was extracted with ethyl acetate (×2). The combined organic layers were washed with saturated solution of sodium bicarbonate, brine, dried over MgSO₄, filtered and evaporated. The residue was purified by silica gel column chromatography using ethyl acetate-n-hexanes 1:2 as eluent to provide the desired product. Yield: 98%. ¹H NMR (CDCl₃): δ 1.38 (t, J=7.0 Hz, 3H), 2.72 (s, 3H), 3.98 (broad s, 2H), 4.40 (q, J=7.2 Hz, 2H), 6.68 (d, J=8.5 Hz, 2H), 7.37 (d, J=8.5 Hz, 2H) ppm. ¹³C NMR (CDCl₃): δ 14.2, 19.2, 62.7, 112.9, 114.6, 115.2, 126.1, 130.9, 136.8, 147.9, 150.1, 156.2, 162.3 ppm. IR: v 3470, 3373, 2233, 1725, 1671, 1625, 1606 cm⁻¹.

### Ethyl 5-cyano-4-methyl-1-(4-(methylamino)phenyl)-6-oxo-1,6-dihydropyridazine-3-carboxylate (C7) and ethyl 5-cyano-1-(4-(dimethylamino)phenyl)-4-methyl-6-oxo-1,6-dihydropyridazine-3-carboxylate (C8)

A mixture of C6 (0.04 g, 0.13 mmol), dimethylsulfate (0.03 g, 25 mL, 0.26 mmol) and potassium carbonate (0.07 g, 0.52 mmol) in acetone (5 mL) was stirred at room temperature for overnight. The reaction mixture was diluted with water and then extracted with etyl acetate. The combined organic layers were washed with brine, dried over MgSO₄, filtered and evaporated. The residue was purified by silica gel column chromatography using ethyl acetate-hexanes 2:3 as eluent to provide the desired compounds. R=4-NMe₂: yield: 33%. ¹H NMR (CDCl₃): δ 1.40 (t, J=7.0 Hz, 3H), 2.74 (s, 3H), 3.02 (s, 6H), 4.41 (q, J=7.2 Hz, 2H), 6.75 (d, J=8.5 Hz, 2H), and 7.51 (d, J=8.5 Hz, 2H) ppm. ¹³C NMR (CDCl₃): δ 14.3, 19.2, 40.6, 62.6, 111.8, 112.9, 115.1, 125.7, 129.4, 136.6, 149.8, 150.8, 156.3, 162.4 ppm. R=4-NHMe: yield: 11%. ¹H NMR (CDCl₃): δ 1.40 (t, J=7.3 Hz, 3H), 2.74 (s, 3H), 2.88 (s, 3H), 4.41 (q, J=7.2 Hz, 2H), 6.63 (d, J=8.5 Hz, 2H), 7.44 (d, J=8.5 Hz, 2H) ppm. ¹³C NMR (CDCl₃): δ 14.2, 19.2, 19.6, 30.6, 62.7, 111.9, 112.9, 115.1, 126.0, 130.0, 136.6, 149.9, 150.1, 156.2, 162.4 ppm.

### Ethyl 1-(3-bromophenyl)-5-cyano-4-methyl-6-oxo-1,6-dihydropyridazine-3-carboxylate (C13)

Prepared as C1 from B10. Yellow oil. ¹H NMR (CDCl₃): δ 1.40 (t, J=7.1 Hz, 3H), 2.75 (s, 3H), 4.43 (q, J=7.1 Hz, H), 7.37 (t, J=8.1 Hz, 1H), 7.06-7.62 (m, 2H), 7.81 (s, 1H) ppm. ¹³C NMR (CDCl₃): δ 14.1, 19.4, 63.0, 112.4, 116.1, 121.3, 122.5, 128.3, 130.4, 132.6, 137.7, 141.0, 155.7, 161.9, 163.1 ppm. IR: v 2236, 1732, 1685, 1585 cm⁻¹. MS [ESI]⁺: calculated for C₁₅H₁₃BrN₃O₃⁺ 362.01 found 361.96.

### Ethyl 5-cyano-4-methyl-6-oxo-1-(m-tolyl)-1,6-dihydropyridazine-3-carboxylate (C14)

Prepared as C1 from B11. Red solid. ¹H NMR (CDCl₃): δ 1.40 (t, J=7.3 Hz, 3H), 2.43 (s, 3H), 2.76 (s, 3H), 4.43 (q, J=7.2 Hz, 2H), 7.28 (d, J=7.6 Hz, 1H), 7.39-7.42 (m, 2H) ppm. ¹³C NMR (CDCl₃): δ 14.3, 19.4, 21.6, 62.9, 112.6, 115.9, 122.3, 125.7, 129.1, 130.4, 137.3, 139.4, 140.1, 150.6, 156.0, 162.2 ppm. IR: v 2236, 1730, 1684, 1601 cm⁻¹. MS [ESI]⁺: calculated for C₁₆H₁₆N₃O₃⁺ 298.12 found 298.21.

### Ethyl 1-(2-chlorophenyl)-5-cyano-4-methyl-6-oxo-1,6-dihydropyridazine-3-carboxylate (C18)

Prepared as C1 from B15. Yellow solid, mp: 93-95° C. (from ethanol). ¹H NMR (CDCl₃): δ 1.38 (t, J=7.1 Hz, 3H), 2.78 (s, 3H), 4.41 (q, J=7.1 Hz, 2H), 7.40-4.49 (m, 3H), 7.57 (dd, J=7.9 and 1.4 Hz, 1H) ppm. ¹³C NMR (CDCl₃): δ 14.3, 19.6, 63.0, 112.3, 115.9, 128.1, 128.8130.8, 131.5, 131.6, 137.6, 137.8, 151.5, 155.4, 162.0 ppm. IR: v 2235, 1731, 1690 cm⁻¹.

### Ethyl 1-(2-bromophenyl)-5-cyano-4-methyl-6-oxo-1,6-dihydropyridazine-3-carboxylate (C19)

Prepared as C1 from B16. White solid, m.p.: 106-108° C. (from ethanol). ¹H NMR (CDCl₃): δ 1.38 (t, J=7.1 Hz, 3H), 2.79 (s, 3H), 4.41 (q, J=7.1 Hz, 2H), 7.38=7.42 (m, 2H), 7.48 (dt, J=7.2 and 1.3 Hz, 1H), 7.74 (dd, J=7.6 and 0.9 Hz, 1H) ppm. ¹³C NMR (CDCl₃): δ 14.3, 19.6, 63.0, 112.3, 116.0, 121.1, 128.8, 128.9, 131.7, 134.0, 137.7, 139.2, 151.5, 155.4, 162.0 ppm. IR: v 2235, 1727, 1689, 1586 cm⁻¹. MS [ESI]⁺: calculated for C₁₅H₁₃BrN₃O₃⁺ 362.01 found 361.93.

### Ethyl 5-cyano-1-(2-(ethoxycarbonyl)phenyl)-4-methyl-6-oxo-1,6-dihydropyridazine-3-carboxylate (C20)

Prepared as C1 from B17. Yellow oil. NMR (CDCl₃): δ 1.27 (t, J=7.1 Hz, 3H), 1.37 (t, J=7.1 Hz, 3H), 2.78 (s, 3H), 4.25 (q, J=7.1 Hz, 2H), 4.39 (q, J=7.1 Hz, 2H), 7.43 (dd, J=7.8 and 0.8 Hz, 1H), 7.59 (dt, J=7.7 Hz and 1.1 Hz, 1H), 7.70 (dt, J=7.7 and 1.4 Hz, 1H), 8.13 (dd, J=7.8 and 1.4 Hz, 1H) ppm. ¹³C NMR (CDCl₃): δ 14.2, 14.3, 61.8, 62.8, 112.5, 115.3, 127.6, 128.2, 130.3, 131.6, 133.6, 137.0, 139.8, 151.4, 156.5, 162.2, 164.4 ppm. IR: v 2234, 1723, 1687, 1603, 1585 cm⁻¹. MS [ESI]⁺: calculated for C₁₈H₁₈N₃O₅⁺ 356.12 found 356.01.

### Ethyl 5-cyano-4-methyl-6-oxo-1-(o-tolyl)-1,6-dihydropyridazine-3-carboxylate (C22)

Prepared as C1 from B19. Yellow oil. ¹H NMR (CDCl₃): δ 1.38 (t, J=7.2 Hz, 3H), 2.18 (s, 3H), 2.78 (s, 3H), 4.40 (q, J=7.2 Hz, 2H), 7.26 (d, J=7.0 Hz, 1H), 7.33-736 (m, 2H), 7.41 (t, J=7.5 Hz, 1H) ppm. ¹³C NMR (CDCl₃): δ 14.2, 17.8, 19.5, 63.2, 112.5, 115.7, 126.9, 127.2, 130.3, 131.5, 134.7, 137.3, 139.2, 151.0, 155.8, 162.2 ppm. IR: v 2234, 1730, 1684 cm⁻¹. MS [ESI]⁺: calculated for C₁₆H₁₆N₃O₃⁺ 298.12 found 297.96.

### Synthesis of Thienyl Derivatives with General Structure D (Gewald Reaction)

### Representative Example: Ethyl 5-amino-3-(4-chlorophenyl)-4-oxo-3,4-dihydrothieno[3,4-d]pyridazine-1-carboxylate (D1)

A mixture of C1 (0.100 g, 0.31 mmol), sulfur (0.015 g, 0.47 mmol), and morpholine (0.55 g, 0.55 mL, 0.63 mmol) was heated to 150° C. using microwave irradiation for 15 min. After cooling, the precipitate which formed was collected and purified by preparative HPLC to give D1 as yellow solid. HPLC-MS retention time 8.20 min. Yield: 64%, mp: 189-191° C. (from ethanol). ¹H NMR (CDCl₃): δ 1.43 (t, J=7.3 Hz, 3H), 4.44 (q, J=7.2 Hz, 2H), 6.21 (broad s, 2H), 7.25 (s, 1H), 7.42 (d, J=8.5 Hz, 2H), 7.56 (d, J=8.5 Hz, 2H) ppm. ¹³C NMR (CDCl₃): δ 14.4, 62.2, 104.9, 106.9, 127.1, 127.2, 128.9, 133.3, 134.0, 139.2, 159.4, 161.8, 163.0 ppm. IR: v 3411, 3303, 1724 cm⁻¹. HRMS (ESI⁺): calculated for C₁₅H₁₃ClN₃O₃S⁺ 350.0366 found 350.0355.

### Ethyl 5-amino-3-(4-bromophenyl)-4-oxo-3,4-dihydrothieno[3,4-d]pyridazine-1-carboxylate (D2)

Was synthesized in the same manner as D1 starting with C2. Yield: 55%, mp: 198-200° C. (from ethanol). ¹H NMR (CDCl₃): δ 1.42 (t, J=7.0 Hz, 3H), 4.44 (q, J=7.2 Hz, 2H), 6.12 (broad s, 2H), 7.25 (s, 1H), 7.51 (d, J=8.5 Hz, 2H), 7.58 (d, J=8.5 Hz, 2H) ppm. ¹³C NMR (CDCl₃): δ 14.1, 62.1, 105.9, 106.7, 121.3, 127.1, 127.5, 131.8, 134.0, 139.7, 159.4, 161.9, 163.0 ppm. IR: v 3407, 3310, 1709, 1657 cm⁻¹. HRMS (ESI⁺): calculated for C₁₅H₁₃BrN₃O₃S⁺ 393.9861 found 393.9879.

### Ethyl 5-amino-3-(4-fluorophenyl)-4-oxo-3,4-dihydrothieno[3,4-d]pyridazine-1-carboxylate (D3)

Was synthesized in the same manner as D1 starting with C3. ¹H-NMR (500 MHz; CDCl₃): δ 1.42 (t, J=7.1 Hz, 3H), 4.44 (q, J=7.1 Hz, 2H), 6.06 (s, 2H), 7.15-7.11 (m, 2H), 7.22 (s, 1H), 7.57-7.54 (m, 2H) ppm.

### Ethyl 5-amino-3-(4-iodophenyl)-4-oxo-3,4-dihydrothieno[3,4-d]pyridazine-1-carboxylate (D4)

Was synthesized as D1 from C4. ¹H-NMR (500 MHz; CDCl₃): δ 1.42 (t, J=7.1 Hz, 3H), 4.44 (q, J=7.1 Hz, 2H), 6.19 (broad s, 2H), 7.25 (s, 1H), 7.40-7.38 (m, 2H), 7.79-7.77 (m, 2H) ppm.

### Ethyl 5-amino-3-(4-azidophenyl)-4-oxo-3,4-dihydrothieno[3,4-d]pyridazine-1-carboxylate (D5)

A mixture of D4 (25 mg, 0.055 mmol), sodium azide (7.2 mg, 0.11 mmol), sodium ascorbate (1 mg), N,N′-dimethylethylendiamine (1.4 mg, 1.8 μL, 0.016 mmol) and cuprous iodide (5 mg, 0.0275 mmol) in dimethylsulfoxide (2.5 mL) and water (0.5 mL) was heated in sealed tube to 60° C. for 1 h. After cooling, the reaction mixture was filtered through a short path of celite, diluted with water and extracted with ethyl acetate. The combined organic layers were washed with brine, dried over sodium sulfate, filtered and evaporated. The residue was purified by silica gel column chromatography using ethyl acetate-hexanes 1:3 as eluent to furnish the desired compound. Yield: 43%, mp: 169-170° C. ¹H-NMR (500 MHz; CDCl₃): δ 1.43 (t, J=7.1 Hz, 3H), 4.44 (q, J=7.1 Hz, 2H), 6.19 (s, 2H), 7.11 (d, J=8.7 Hz, 2H), 7.25 (s, 1H), 7.61 (d, J=8.7 Hz, 2H) ppm.

### Ethyl 5-amino-3-(4-nitrophenyl)-4-oxo-3,4-dihydrothieno[3,4-d]pyridazine-1-carboxylate (D6)

Was synthesized as D1 from C5. Yield: 43%. ¹HNMR (DMSO-d₆): δ 1.33 (t, J=7.0 Hz, 3H), 4.36 (q, J=7.1 Hz, 2H), 7.12 (s, 1H), 7.78 (broad s, 2H), 7.90 (d, J=8.5 Hz, 2H), 8.35 (d, J=8.5 Hz, 2H) ppm. ¹³C NMR (DMSO-d₆): δ 14.6, 62.2, 103.9, 105.2, 124.5, 126.2, 126.6, 134.6, 145.9, 146.6, 158.7, 162.7, 164.6 ppm.

### Ethyl 5-amino-3-(4-aminophenyl)-4-oxo-3,4-dihydrothieno[3,4-d]pyridazine-1-carboxylate (D7)

Was synthesized as D1 from C6. Yield: 77%. ¹H NMR (DMSO-d₆): δ 1.29 (t, J=7.0 Hz, 3H), 4.30 (q, J=7.0 Hz, 2H), 5.27 (broad s, 2H), 6.60 (d, J=8.5 Hz, 2H), 7.05-7.07 (m, 3H), 7.55 (broad s, 2H) ppm. ¹³C NMR (DMSO-d₆): δ 14.7, 61.8, 103.1, 105.0, 113.7, 126.9, 127.8, 129.9, 132.51, 148.9, 159.2, 163.1, 163.3 ppm. IR: v 3424, 3319, 3259, 3160, 1724, 1704, 1637, 1605 cm⁻¹.

### Ethyl 5-amino-3-(4-(methylamino)phenyl)-4-oxo-3,4-dihydrothieno[3,4-d]pyridazine-1-carboxylate (D8)

Was synthesized as D1 from C7. Yield: 81%; mp: 211-213° C. (from ethanol). ¹H NMR (CDCl₃): δ 1.29 (t, J=7.0 Hz, 3H), 2.71 (s, 3H), 4.30 (q, J=7.2 Hz, 2H), 5.84 (broad s, 1H), 6.57 (d, J=8.5 Hz, 2H), 7.06 (s, 1H), 7.15 (d, J=8.5 Hz, 2H), 7.55 (broad s, 2H) ppm.

### Ethyl 5-amino-3-(4-(dimethylamino)phenyl)-4-oxo-3,4-dihydrothieno[3,4-d]pyridazine-1-carboxylate (D9)

Was synthesized as D1 from C8. Yield: 81%; mp: 211-213° C. (from ethanol). ¹H NMR (CDCl₃): δ 1.29 (t, J=7.0 Hz, 3H), 2.93 (s, 6H), 4.31 (q, J=6.7 Hz, 2H), 6.76 (d, J=8.5 Hz, 2H), 7.07 (s, 1H), 7.25 (d, J=8.5 Hz, 2H), 7.56 (broad s, 2H) ppm.

### Ethyl 5-amino-4-oxo-3-(4-(trifluoromethoxy)phenyl)-3,4-dihydrothieno[3,4-d]pyridazine-1-carboxylate (D10)

Was synthesized as D1 from C11. Yield: 55%. ¹H-NMR (500 MHz; CDCl₃): δ 1.43 (t, J=7.1 Hz, 3H), 4.45 (q, J=7.1 Hz, 2H), 7.26 (s, 1H), 7.31 (d, J=8.5 Hz, 2H), 7.67 (d, J=2.0 Hz, 2H) ppm.

### Ethyl 5-amino-3-(4-hydroxyphenyl)-4-oxo-3,4-dihydrothieno[3,4-d]pyridazine-1-carboxylate (D11)

Was synthesized as D1 from C10. Yield: 46%. ¹H NMR (DMSO-d₆): δ 1.29 (t, J=7.2 Hz, 3H), 4.31 (q, J=7.0 Hz, 2H), 6.83 (d, J=8.5 Hz, 2H), 7.07 (s, 1H), 7.25 (d, J=9.0 Hz, 2H), 7.57 (broad s, 2H), 9.68 (broad s, 1H) ppm. ¹³C NMR (DMSO-d₆): δ 14.7, 61.9, 103.5, 104.8, 115.5, 126.8, 128.3, 132.8, 157.3, 159.1, 163.1, 163.5. IR: v 3404, 1639 cm⁻¹. MS [ESI]⁺: calculated for C₁₅H₁₄N₃O₄S⁺ 332.07 found 332.04.

### Ethyl 5-amino-4-oxo-3-(4-(trifluoromethyl)phenyl)-3,4-dihydrothieno[3,4-d]pyridazine-1-carboxylate (D12)

Was synthesized as D1 from C11. Yield: 90%. ¹H-NMR (500 MHz; CDCl₃): δ 1.43 (t, J=7.1 Hz, 3H), 4.45 (q, J=7.1 Hz, 2H), 7.27 (s, 1H), 7.72 (d, J=8.6 Hz, 2H), 7.80 (d, J=8.4 Hz, 2H) ppm.

### Ethyl 5-amino-3-(4-nitrophenyl)-4-oxo-3,4-dihydrothieno[3,4-d]pyridazine-1-carboxylate (D13)

Was synthesized as D1 from C12. Yield: 43%. ¹H NMR (DMSO-d₆): δ 1.33 (t, J=7.0 Hz, 3H), 4.36 (q, J=7.1 Hz, 2H), 7.12 (s, 1H), 7.78 (broad s, 2H), 7.90 (d, J=8.5 Hz, 2H), 8.35 (d, J=8.5 Hz, 2H) ppm. ¹³C NMR (DMSO-d₆): δ 14.6, 62.2, 103.9, 105.2, 124.5, 126.2, 126.6, 134.6, 145.9, 146.6, 158.7, 162.7, 164.6 ppm.

### Ethyl 5-amino-3-(3-bromophenyl)-4-oxo-3,4-dihydrothieno[3,4-d]pyridazine-1-carboxylate (D14)

Was synthesized as D1 from C13. Yield: 78%; mp: 163-165° C. (from dichloromethane). ¹H NMR (CDCl₃): δ 1.43 (t, J=7.1 Hz, 3H), 4.45 (q, J=7.5 Hz, 2H), 6.11 (broad s, 2H), 7.32 (t, J=8.05 Hz, 1H), 7.48 (d, J=7.4 Hz, 1H), 7.58 (d, J=7.9 Hz, 1H), 7.80 (s, 1H) ppm. ¹³C NMR (CDCl₃): δ 14.5, 62.3, 105.1, 106.8, 122.2, 124.7, 127.1, 129.2, 130.0, 130.8, 134.2, 141.8, 159.4, 162.0, 163.1 ppm. IR: v 3419, 3320, 1709, 1646, 1596 cm⁻¹. HRMS [ESI]⁺: calculated for C₁₅H₁₃BrN₃O₃S⁺ 393.9861 found 393.9857.

### Ethyl 5-amino-4-oxo-3-(m-tolyl)-3,4-dihydrothieno[3,4-d]pyridazine-1-carboxylate (D15)

Was synthesized as D1 from C14. Yield: 81%; mp: 190-191° C. (from dichloromethane). ¹H NMR (CDCl₃): δ 1.42 (t, J=7.1 Hz, 3H), 2.41 (s, 3H), 4.44 (q, J=7.1 Hz, 2H), 6.21 (broad s, 2H), 7.17 (d, J=6.6 Hz, 1H), 7.22 (s, 1H), 7.32-7.38 (m, 3H) ppm. ¹³C NMR (CDCl₃): δ 14.4, 21.6, 62.1, 104.1, 17.2, 123.4, 126.9, 127.4, 128.7, 128.8, 133.7, 138.9, 140.6, 159.7, 161.7, 163.3 ppm. IR: v 3436, 3329, 1706, 1652, 1598 cm⁻¹. HRMS [ESI]⁺: calculated for C₁₆H₁₆N₃O₃S⁺ 330.0929 found 330.0912.

### Ethyl 5-amino-3-(2-chlorophenyl)-4-oxo-3,4-dihydrothieno[3,4-d]-pyridazine-1-carboxylate (D16)

It was synthesized as D1 from C18. Yield: 87%. ¹H NMR (CDCl₃): δ 1.27 (m, 3H), 4.29-4.33 (m, 2H), 7.15-7.16 (m, 1H), 7.47 (m, 2H), 7.53-7.60 (m, 3H), 7.64-7.67 (m, 1H) ppm. ¹³C NMR (CDCl₃): δ 14.0, 54.9, 61.4, 103.5, 104.1, 126.0, 128.0, 129.7, 130.4, 130.6, 131.7, 133.0, 138.3, 157.9, 162.2, 163.1 ppm. IR: v 3425, 3303, 1710, 1648, 1587 cm⁻¹. HRMS [ESI]⁺: calculated for C₁₅H₁₂ClNaN₃O₃S 372.0186 found 372.0190.

### Ethyl 5-amino-3-(3-(ethoxycarbonyl)phenyl)-4-oxo-3,4-dihydrothieno[3,4-d]pyridazine-1-carboxylate (D17)

Was synthesized as D1 from C16. Yield: 73%; mp: 159-160° C. (from dichloromethane). ¹H NMR (CDCl₃): δ 1.38-1.43 (m, 6H), 4.37-4.46 (m, 4H), 6.36 (broad s, 2H), 7.22 (s, 1H), 7.53 (t, J=7.9 Hz, 1H), 7.81 (d, J=7.95 Hz, 1H), 8.03 (d, J=7.8 Hz, 1H), 8.29 (s, 1H) ppm. ¹³C NMR (CDCl₃): δ 14.4, 14.5, 6.1.4, 61.2, 104.8, 106.6, 127.2, 127.3, 128.7, 128.9, 130.4, 131.2, 134.1, 140.9, 159.5, 162.1, 163.1, 166.2 ppm. IR: n 3452, 3345, 1721, 1667, 1600 cm⁻¹. HRMS [ESI]⁺: calculated for C₁₈H₁₇N₃NaO₅S⁺ 410.0787 found 410.0781.

### Ethyl 5-amino-3-(2-fluorophenyl)-4-oxo-3,4-dihydrothieno[3,4-d]pyridazine-1-carboxylate (D18)

Was synthesized as D1 from C17. NMR (CDCl₃): δ 1.41 (t, J=7.1 Hz, 3H), 4.44 (q, J=7.1 Hz, 2H), 6.16 (broad s, 2H), 7.20-7.31 (m, 2H), 7.38-7.43 (m, 1H), 7.48 (dt, J=7.65 and 1.5 Hz, 1H) ppm.

### Ethyl 5-amino-3-(2-chlorophenyl)-4-oxo-3,4-dihydrothieno[3,4-d]pyridazine-1-carboxylate (D19)

Was synthesized as D1 from C18. NMR (CDCl₃): δ 1.27 (m, 3H), 4.29-4.33 (m, 2H), 7.15-7.16 (m, 1H), 7.47 (m, 2H), 7.53-7.60 (m, 3H), 7.64-7.67 (m, 1H) ppm.

### Ethyl 5-amino-3-(2-bromophenyl)-4-oxo-3,4-dihydrothieno[3,4-d]pyridazine-1-carboxylate (D20)

Was synthesized as D1 from C19. Yield: 78%; mp: 203-205° C. (from dichloromethane). ¹H NMR (CDCl₃): δ 1.40 (t, J=7.0 Hz, 3H), 4.43 (q, J=7.0 Hz, 2H), 6.17 (broad s, 2H), 7.26 (s, 1H), 7.31 (t, J=7.3 Hz, 1H), 7.42-7.47 (m, 2H), 7.71 (d, J=8.0 Hz, 1H) ppm. ¹³C NMR (CDCl₃): δ 14.4, 62.2, 105.3, 106.8, 123.0, 127.4, 128.6, 130.3, 130.6, 133.7, 140.2, 139.9, 159.3, 161.7, 163.1 ppm. IR: v 3424, 3305, 1721, 1659, 1585 cm⁻¹. HRMS [ESI]⁺: calculated for C₁₅H₁₃BrN₃O₃S⁺ 393.9861 found 393.9879.

### Ethyl 5-amino-3-(2-(ethoxycarbonyl)phenyl)-4-oxo-3,4-dihydrothieno[3,4-d]pyridazine-1-carboxylate (D21)

Was synthesized as D1 from C20. Yield: 78%; mp: 176-178° C. (from dichloromethane). ¹H NMR (CDCl₃): δ 1.10 (t, J=7.1 Hz, 3H), 1.38 (t, J=7.1 Hz, 3H), 4.19 (q, J=7.1H, 2H), 4.40 (q, J=7.1 Hz, 2H), 6.21 (broad s, 2H), 7.21 (s, 1H), 7.46-7.52 (m, 2H), 7.63 (t, J=7.6 Hz, 1H), 8.03 (d, J=7.8 Hz, 1H) ppm. ¹³C NMR (CDCl₃): δ 13.9, 14.4, 61.4, 62.1, 104.7, 106.8, 127.4, 128.7, 128.9, 129.2, 131.2, 133.0, 133.5, 140.1, 160.0, 161.8, 163.2, 165.7 ppm. IR: v 3417, 1712, 1656, 1593 cm⁻¹. HRMS [ESI]⁺: calculated for C₁₈H₁₇N₃NaO₅S⁺ 410.0787 found 410.0799.

### Ethyl 3-([1,1′-biphenyl]-2-yl)-5-amino-4-oxo-3,4-dihydrothieno[3,4-d]pyridazine-1-carboxylate (D22)

Was synthesized as D1 from C21. ¹H NMR (CDCl₃): δ 1.36 (t, J=7.1 Hz, 3H), 4.36 (broad s, 2H), 6.03 (broad s, 2H), 7.15 (s, 1H), 7.19-7.35 (m, 5H), 7.44-7.52 (m, 4H) ppm.

### Ethyl 5-amino-4-oxo-3-(o-tolyl)-3,4-dihydrothieno[3,4-d]pyridazine-1-carboxylate (D23)

Was synthesized as D1 from C22. Yield: 75%; mp: 209-210° C. (from dichloromethane). ¹H NMR (DMSO-d₆): δ 1.28 (t, J=7.2 Hz, 3H), 2.11 (s, 3H), 4.30 (q, J=7.1 Hz, 2H), 7.13 (s, 1H), 7.31-7.37 (m, 4H), 7.57 (broad s, 2H) ppm. ¹³C NMR (DMSO-d₆): δ 14.0, 17.2, 61.3, 103.4, 104.0, 126.2, 126.5, 128.3, 128.5, 130.5, 132.6, 135.4, 139.9, 158.3, 162.4, 162.9 ppm. IR: v 3429, 3317, 3137, 1717, 1641, 1585 cm⁻¹. HRMS [ESI]⁺: calculated for C₁₆H₁₅NaN₃O₃S⁺ 352.0732 found 352.0736.

### Synthesis of Compounds with General Structure E (Esters Hydrolysis)

### Representative Example: 5-Amino-3-(4-chlorophenyl)-4-oxo-3,4-dihydrothieno[3,4-d]pyridazine-1-carboxylic acid (E1)

Lithium hydroxide monohydrate (0.063 g, 1.29 mmol) was added to a solution of D1 (0.150 g, 0.43 mmol) in tetrahydrofuran (3 mL) and water (2 mL). The reaction mixture was stirred at room temperature for 16 h. 1 N HCl was added (pH ˜2), the formed precipitate was filtered and purified by preparative HPLC to afford E1 as yellow solid. HPLC-MS retention time 6.63 min. Yield: 67%, mp: >300° C. (from ethanol). ¹H NMR (CD₃OD): δ 7.19 (s, 1H), 7.44 (d, J=9.0 Hz, 2H), 7.58 (d, J=9.0 Hz, 2H) ppm. ¹³C NMR (DMSO-d₆): δ 104.4, 104.5, 126.8, 128.3, 128.9, 132.0, 134.5, 140.2, 158.9, 163.9, 164.6 ppm. IR: v 3439, 3322, 3065, 1712, 1646, 1590 cm⁻¹. HRMS (ESI⁺): calculated for C₁₃H₉ClN₃O₃S⁺ 322.0053 found 322.0062.

### 5-Amino-3-(4-bromophenyl)-4-oxo-3,4-dihydrothieno[3,4-d]pyridazine-1-carboxylic acid (E2)

Prepared in the same manner as E1 starting with D2. HPLC-MS retention time 6.76 min. Yield: 77%, mp: >300° C. (from ethanol). ¹H NMR (DMSO-d₆): δ 7.12 (s, 1H), 7.52 (d, J=9.0 Hz, 2H), 7.63 (broad s, 2H), 7.66 (d, J=8.5 HZ, 2H) ppm. ¹³C NMR (DMSO-d₆): δ 104.4, 104.5, 120.4, 126.8, 128.6, 131.9, 134.5, 140.6, 158.8, 163.9, 164.5 ppm. IR: v 3439, 3322, 3065, 1712, 1646 cm⁻¹. HRMS (ESI⁺): calculated for C₁₃H₇BrN₃NaO₃S⁺ 387.9367 found 387.9378.

### 5-Amino-3-(4-fluorophenyl)-4-oxo-3,4-dihydrothieno[3,4-d]pyridazine-1-carboxylic acid (E3)

Prepared in the same manner as E1 starting with D3. ¹H NMR (DMSO-d₆): δ 7.12 (s, 1H), 7.29 (t, J=8.7 Hz, 2H), 7.55-7.58 (m, 4H) ppm.

### 5-Amino-3-(4-hydroxyphenyl)-4-oxo-3,4-dihydrothieno[3,4-d]pyridazine-1-carboxylic acid (E4)

Prepared in the same manner as E1 starting with D11. ¹H-NMR (500 MHz; DMSO-d₆): δ 6.81 (d, J=8.8 Hz, 2H), 7.08 (s, 1H), 7.26 (d, J=8.8 Hz, 2H), 7.55 (broad s, 2H), 9.65 (s, 1H) ppm.

### 5-Amino-4-oxo-3-(4-(trifluoromethoxy)phenyl)-3,4-dihydrothieno[3,4-d]pyridazine-1-carboxylic acid (E5)

Prepared in the same manner as E1 starting with D10. ¹H-NMR (500 MHz; MeOD): δ 7.17 (s, 1H), 7.35 (d, J=8.5 Hz, 2H), 7.69 (d, J=8.9 Hz, 2H) ppm.

### 5-Amino-3-(3-chlorophenyl)-4-oxo-3,4-dihydrothieno[3,4-d]pyridazine-1-carboxylic acid (E6)

Prepared in the same manner as E1 starting with D16. ¹H-NMR (500 MHz; DMSO-d₆): δ 7.13 (s, 1H), 7.44-7.42 (m, 1H), 7.50 (t, J=8.0 Hz, 1H), 7.55 (d, J=8.0 Hz, 1H), 7.68-7.65 (m, 3H) ppm.

### Isopropyl 5-amino-3-(4-chlorophenyl)-4-oxo-3,4-dihydrothieno[3,4-d]pyridazine-1-carboxylate (2)

A mixture of D1 (0.050 g, 0.143 mmol) and titanium (IV) isopropoxide (0.002 g, 2.1 μL, 7 μmol) in isopropanol (0.5 mL) was heated to 170° C. using microwave irradiation for 40 min. After cooling, the solvent was evaporated and the residue was purified by preparative HPLC to provide 2 as yellow solid. HPLC-MS retention time 8.73 min. Yield: 77%, mp: 118-120° C. (from ethanol). ¹H NMR (CDCl₃): δ 1.41 (d, J=6.5 Hz, 6H), 5.24-5.33 (m, 1H), 6.25 (broad s, 2H), 7.17 (s, 1H), 7.40 (d, J=8.5 Hz, 2H), 7.57 (d, J=8.5 Hz, 2H) ppm. ¹³C NMR (CDCl₃): δ 22.0, 70.1, 104.7, 106.8, 127.1, 127.2, 128.8, 133.1, 134.4, 139.2, 159.4, 161.9, 162.5 ppm. IR: v 3408, 3294, 3145, 1716, 1663, 1585 cm⁻¹. HRMS (ESI⁺): calculated for C₁₆H₁₄ClN₃O₃NaS⁺ 386.0342 found 386.0331.

### Synthesis of Compounds with General Structure F

### Representative Example: 5-Amino-3-(4-chlorophenyl)-N-isopropyl-4-oxo-3,4-dihydrothieno[3,4-d]pyridazine-1-carboxamide (F3)

DIPEA (0.012 g, 14 μL, 0.093 mmol) was added to a mixture of E1 (0.020 g, 0.062 mmol), isopropylamine (0.007 g, 11 μL, 0.124 mmol), and BOP reagent (0.041 g, 0.093 mmol) in anhydrous DMSO (1 mL). The reaction mixture was stirred at room temperature for 4 h. Water was added and the resulting mixture was extracted with ethyl acetate. The organic layer was washed with brine, dried over MgSO₄, filtered and evaporated. The residue was purified by preparative HPLC to give the F3 as yellow solid. HPLC-MS retention time: 8.04 min. Yield: 89%, mp: 197-199° C. (from ethanol). ¹H NMR (CD₃OD): δ 1.22 (d, J=6.5 Hz, 6H), 4.09-4.19 (m, 1H), 7.25 (s, 1H), 7.46 (d, J=8.5 Hz, 2H), 7.59 (d, J=8.5 Hz, 2H) ppm. ¹³C NMR (CD₃OD): δ 19.6, 39.9, 102.9, 103.3, 124.9, 126.1, 126.9, 131.2, 135.1, 138.1, 158.0, 161.4, 162.1 ppm. IR: v 3404, 3294, 3178, 1661 cm⁻¹. HRMS (ESI⁺): calculated for C₁₆H₁₅ClN₄O₂NaS⁺ 385.0502 found 385.0492.

### 5-Amino-3-(4-chlorophenyl)-N-methyl-4-oxo-3,4-dihydrothieno[3,4-d]pyridazine-1-carboxamide (F1)

Prepared in the same manner as F3 starting with E1 using methylamine (2.0 M solution in tetrahydrofuran). HPLC-MS retention time: 6.83 min. Yield: 52%, mp: 192-194° C. (from ethanol). ¹H NMR (DMSO-d₆): δ 2.75 (d, J=5.0 Hz, 3H), 7.24 (s, 1H), 7.51 (d, J=9.0 Hz, 2H), 7.60 (broad s, 2H), 7.69 (d, J=9 Hz, 2H), 8.27 (broad s, 1H) ppm. ¹³C NMR (DMSO-d₆): δ 26.4, 104.5, 105.2, 126.6, 128.1, 128.8, 131.6, 136.8, 140.1, 158.9, 163.5, 163.6 ppm. IR: v 3418, 3302, 1656, 1593 cm⁻¹. HRMS (ESI⁺): calculated for C₁₄H₁₀ClN₄O₂S⁺ 333.0213 found 333.0210.

### 5-Amino-3-(4-chlorophenyl)-N-ethyl-4-oxo-3,4-dihydrothieno[3,4-d]pyridazine-1-carboxamide (F2)

Prepared in the same manner as F3 starting with E1 using ethylamine hydrochloride. HPLC-MS retention time: 7.43 min. Yield: 58%, mp: 203-205° C. (from ethanol). ¹H NMR (CDCl₃): δ 1.23 (t, J=7.3 Hz, 3H), 341-347 (m, 2H), 6.18 (broad s, 2H), 7.07 (s, 1H), 7.45 (d, J=9 Hz, 2H), 7.51 (d, J=9 Hz, 2H), 7.60 (broad s, 1H) ppm. ¹³C NMR (CDCl₃): δ 14.9, 34.3, 106.8, 106.9, 126.7, 127.3, 129.0, 133.3, 135.6, 139.2, 159.5, 161.4, 162.6 ppm. IR: v 3405, 3297, 1652, 1600 cm⁻¹. HRMS (ESI⁺): calculated for C₁₅H₁₃ClN₄NaO₂S⁺ 371.0345 found 371.0341.

### 5-Amino-3-(4-chlorophenyl)-N-cyclopropyl-4-oxo-3,4-dihydrothieno[3,4-d]pyridazine-1-carboxamide (F4)

Prepared in the same manner as F3 starting with E1 using cyclopropylamine. HPLC-MS retention time: 7.43 min. Yield: 41%, mp: 188-190° C. (from ethanol). ¹H NMR (CD₃OD): δ 0.58-0.60 (m, 2H), 0.66-0.69 (m, 2H), 2.76-2.81 (m, 1H), 7.19 (s, 1H), 7.50, (d, J=9.0 Hz, 2H), 7.67 (d, J=9.0 Hz, 2H), 8.28 (broad s, 1H) ppm. ¹³C NMR (CD₃OD): δ 6.3, 23.2, 104.4, 105.1, 126.6, 128.2, 128.7, 131.6, 137.0, 140.1, 158.9, 163.3, 164.4 ppm. IR: v 3408, 3302, 1654, 1593 cm⁻¹. HRMS (ESI⁺): calculated for C₁₆H₁₃Cl₄O₂N₄NaS⁺ 383.0345 found 383.0333.

### 5-Amino-3-(4-chlorophenyl)-N,N-dimethyl-4-oxo-3,4-dihydrothieno[3,4-d]pyridazine-1-carboxamide (F5)

Prepared in the same manner as F3 starting with E1 using dimethylamine hydrochloride. HPLC-MS retention time: 6.43 min. Yield: 62%, mp: 242-244° C. (from ethanol). ¹H NMR (CDCl₃): δ 3.14 (s, 3H), 3.16 (s, 3H), 6.18 (broad s, 2H), 6.73 (s, 1H), 7.41 (d, J=9.0 Hz, 2H), 7.52 (d, J=9.0 Hz, 2H) ppm. ¹³C NMR (CD₃OD): δ 35.7, 39.0, 104.0, 106.9, 127.0, 128.1, 128.8, 132.9, 139.2, 139.3, 159.1, 161.7, 164.3 ppm. IR: v 3414, 3300, 1646, 1598 cm⁻¹. HRMS (ESI⁴): calculated for C₁₄H₁₃ClN₄NaO₂S⁺ 371.0345 found 371.0366.

### 5-Amino-3-(4-chlorophenyl)-N-ethyl-N-methyl-4-oxo-3,4-dihydrothieno[3,4-d]pyridazine-1-carboxamide (F6)

Prepared in the same manner as F3 starting with E1 using ethylmethylamine. HPLC-MS retention time 6.85 min. Yield: 47%, mp: 148-150° C. (from ethanol). ¹H NMR (CD₃OD): δ 1.19-1.26 (m, 3H), 3.48-3.60 (m, 2H), 6.16 (broad s, 2H), 6.70 (s, 1H), 7.40-7.42 (m, 2H), 7.51-7.54 (m, 2H) ppm. ¹³C NMR (CD₃OD): δ 12.2, 14.0, 32.9, 36.3, 42.9, 46.1, 103.5, 106.4, 111.3, 117.0, 126.7, 126.8, 127.0, 127.1, 127.8, 127.9, 128.8 (×2), 132.8, 132.9, 159.1 (×2), 162.2, 164.0, 164.5 ppm. IR: v 3409, 3290, 3175, 1635 cm⁻¹. HRMS (ESI⁺): calculated for C₁₆H₁₅ClN₄NaO₂S⁺ 385.0502 found 385.0491.

### 5-Amino-3-(4-bromophenyl)-N-isopropyl-4-oxo-3,4-dihydrothieno[3,4-d]pyridazine-1-carboxamide (F7)

Prepared in the same manner as F3 starting with E2. HPLC-MS retention time 8.20 min. Yield: 68%, mp: 222° C. dec (from ethanol). ¹H NMR (CD₃OD): δ 1.26 (d, J=5.0 Hz, 6H), 4.13-4.22 (m, 1H), 7.29 (s, 1H), 7.56 (d, J=8.5 Hz, 2H), 7.64 (d, J=8.5 Hz, 2H) ppm. ¹³C NMR (CD₃OD): δ 19.6, 58.6, 103.0, 103.3, 119.0, 124.9, 126.3, 129.9, 135.1, 138.6, 157.9, 161.4, 162.1 ppm. IR: v 3412, 3305, 1655, 1598 cm⁻¹. HRMS (ESI⁺): calculated for C₁₆H₁₅BrN₄NaO₂S⁺ 428.9997 found 428.9986.

### 5-Amino-3-(4-fluorophenyl)-N-isopropyl-4-oxo-3,4-dihydrothieno[3,4-d]pyridazine-1-carboxamide (F8)

Prepared in the same manner as F3 starting with E3. ¹H-NMR (500 MHz; CDCl₃): δ 1.24 (d, J=6.5 Hz, 6H), 4.21 (dt, J=13.5, 6.7 Hz, 1H), 6.90 (d, J=7.4 Hz, 1H), 7.16 (t, J=8.4 Hz, 2H), 7.51 (dd, J=8.3, 4.8 Hz, 2H), 7.58 (s, 1H) ppm.

### 5-Amino-N-cyclopropyl-3-(4-fluorophenyl)-4-oxo-3,4-dihydrothieno[3,4-d]pyridazine-1-carboxamide (F9)

Prepared in the same manner as F3 starting with E3 and cyclopropyl amine. ¹H-NMR (500 MHz; DMSO-d₆): δ 0.58 (m, 2H), 0.69-0.66 (m, 2H), 2.78 (m, 1H), 7.19 (s, 1H), 7.30-7.26 (m, 2H), 7.57 (s, 2H), 7.66-7.63 (m, 2H), 8.25 (d, J=4.2 Hz, 1H) ppm.

### 5-Amino-N,N-diethyl-3-(4-methoxyphenyl)-4-oxo-3,4-dihydrothieno[3,4-d]pyridazine-1-carboxamide (F10)

Prepared in the same manner as F3 starting with 6b and diethylamine. ¹H MNR (CDCl₃): δ 1.18 (t, J=7.1 Hz, 3H), 1.26 (t, J=7.1 Hz, 3H), 3.45 (q, J=7.1 Hz, 2H), 3.55 (q, J=7.1 Hz, 2H), 3.83 (s, 3H), 6.20 (broad s, 2H), 6.64 (s, 1H), 6.95 (d, J=8.8 Hz, 2H), 7.42 (d, J=8.8 Hz, 2H) ppm.

### 5-Amino-3-(3-chlorophenyl)-N-isopropyl-4-oxo-3,4-dihydrothieno[3,4-d]pyridazine-1-carboxamide (F11)

Prepared in the same manner as F3 starting with E6. ¹H-NMR (500 MHz; MeOD): δ 1.27 (d, J=6.6 Hz, 6H), 4.16-4.12 (m, 1H), 7.28 (s, 1H), 7.38 (dt, J=8.0, 0.9 Hz, 1H), 7.46 (t, J=8.0 Hz, 1H), 7.58 (dt, J=8.0, 0.9 Hz, 1H), 7.71 (d, J=1.9 Hz, 1H) ppm.

### 5-Amino-N-isopropyl-4-oxo-3-(4-(trifluoromethoxy)phenyl)-3,4-dihydrothieno[3,4-c]pyridazine-1-carboxamide (F12)

Prepared in the same manner as F3 starting with E5. ¹H-NMR (500 MHz; CDCl₃): δ 1.25 (d, J=6.6 Hz, 6H), 4.25-4.18 (m, 1H), 6.19 (s, 2H), 6.89 (d, J=7.9 Hz, 1H), 7.34 (d, J=8.2 Hz, 2H), 7.62-7.60 (m, 3H) ppm.

### 5-Amino-3-(4-hydroxyphenyl)-N-isopropyl-4-oxo-3,4-dihydrothieno[3,4-d]pyridazine-1-carboxamide (F13)

Prepared in the same manner as F3 starting with E4. ¹H-NMR (500 MHz; DMSO-d₆): δ 1.14 (d, J=6.6 Hz, 6H), 4.07-4.02 (m, 1H), 6.82 (d, J=8.8 Hz, 2H), 7.16 (s, 1H), 7.34 (d, J=8.8 Hz, 2H), 7.50 (broad s, 2H), 7.85 (d, J=8.1 Hz, 1H), 9.61 (s, 1H) ppm.

### 5-Amino-3-(4-chlorophenyl)-N-(2-hydroxyethyl)-4-oxo-3,4-dihydrothieno[3,4-d]pyridazine-1-carboxamide (F14)

Prepared in the same manner as F3 starting with E1 and ethanolamine. ¹H MNR (DMSO-d₆): δ 3.29-332 (m, 2H), 3.48 (t, J=6.1 Hz, 2H), 4.77 (broad s, 1H), 7.25 (s, 1H), 7.52 (d, J=7.4 Hz, 2H), 7.62 (broad s, 2H), 7.68 (d, J=7.7 Hz, 2H), 8.21 (t, J=5.7 Hz, 1H) ppm.

### 5-Amino-3-(4-chlorophenyl)-4-oxo-3,4-dihydrothieno[3,4-d]pyridazine-1-carbohydrazide (F15)

D1, dissolved in 1 ml EtOH was added with ˜30 eq. of hydrazine monohydrate (˜240 ult). The resulting mixture was heated at 160° C. for 1 h under microwave promoted conditions. After evaporation of volatiles the crude material was purified by preparative HPLC obtaining the title compound. ¹H-NMR (500 MHz; DMSO-d₆): δ 4.51 (broad s, 2H), 7.12 (s, 1H), 7.51-7.47 (m, 2H), 7.61 (broad s, 2H), 7.75-7.72 (m, 2H), 9.62 (s, 1H) ppm.

### Ethyl 3-(4-chlorophenyl)-5-(methylamino)-4-oxo-3,4-dihydrothieno[3,4-d]pyridazine-1-carboxylate (CNDR-51360)

A mixture of 17, potassium carbonate and iodometane in acetonitrile was put in a vial and stirred and heated at 70° C. for 10 h. After cooling the reaction mixture was diluted with water and then extracted with ethyl acetate. Organic layer was washed with brine, dried over MgSO4, filtered and evaporated. The residue was purified by silica gel column chromatography using ethyl acetate-n-Hexane 1:3 as eluent to give the title compound. MS (ESI⁺): calculated for C₁₆H₁₅ClN₃O₃S⁺ 364.05 found 364.07.

### Ethyl 5-amino-7-chloro-3-(4-chlorophenyl)-4-oxo-3,4-dihydrothieno[3,4-d]pyridazine-1-carboxylate (CNDR-51393)

A mixture 17 (30 mg, 0.086 mmol) and N-chlorosuccinimide (11 mg, 0.086 mmol) in anhydrous N,N-dimethylformamide (0.5 mL) was heated to 80° C. for 30 minutes. After cooling, water was added and the resulting mixture was extracted with ethyl acetate. The combined organic layers were washed with brine, dried over sodium sulfate, filtered and evaporated. The residue was purified by preparative HPLC using a gradient of acetonitrile in water from 10% to 90% in 10 minutes to give the desired compound. ¹H-NMR (500 MHz; CDCl₃): δ 1.42 (t, J=7.2 Hz, 3H), 4.45 (q, J=7.2 Hz, 2H), 6.27 (broad s, 2H), 7.41 (d, J=8.8 Hz, 2H), 7.53-7.51 (m, 2H) ppm.

### Ethyl 5-amino-7-bromo-3-(4-chlorophenyl)-4-oxo-3,4-dihydrothieno[3,4-d]pyridazine-1-carboxylate (CNDR-51411)

Prepared in the same manner as CNDR-51393 starting with 17 and N-bromosuccinimide. ¹H-NMR (500 MHz; CDCl₃): δ 1.43 (t, J=7.2 Hz, 3H), 4.46 (q, J=7.2 Hz, 2H), 6.34 (broad s, 2H), 7.40 (d, J=8.8 Hz, 2H), 7.52 (d, J=8.8 Hz, 2H) ppm.

### Ethyl (1-(4-chlorophenyl)-5-cyano-4-methyl-6-oxo-1,6-dihydropyridazin-3-yl)carbamate (CNDR-51385)

A mixture of 11 (30 mg, 0.095 mmol) in absolute ethanol (2 mL) was refluxed for 5 h. The reaction mixture was cooled at room temperature and diluted with water. The resulting mixture was extracted with ethyl acetate and the combined organic layers were washed with brine, dried over sodium sulfate, filtered and evaporated. The residue was purified by silica gel column chromatography using ethyl acetate-hexanes 2:3 as eluent to provide the desired compound. ¹H NMR (CDCl₃): δ 1.33 (t, J=7.0 Hz, 3H), 4.25 (q, J=7.2 Hz, 2H), 6.17 (broad s, 2H), 6.70 (s, 1H), 6.75 (broad s, 1H), 7.39 (d, J=8.5 Hz, 2H), 7.58 (d, J=8.5 Hz, 2H) ppm.

### tert-Butyl (1-(4-chlorophenyl)-5-cyano-4-methyl-6-oxo-1,6-dihydropyridazin-3-yl)carbamate (CNDR-51383).)

Prepared in the same manner as CNDR-51385 starting with 11 and tert-butanol. ¹H NMR (CDCl₃): δ 1.52 (s, 9H), 6.18 (broad s, 2H), 6.63 (broad s, 1H), 6.72 (s, 1H), 7.38 (d, J=8.5 Hz, 2H), 7.57 (d, J=8.5 Hz; 2H) ppm.

### Characterization of Novel ATPZ Inhibitors of Tau Assembly

Compounds 5a and 5b were identified as inhibitors of tau fibril assembly during the screening of a large compound library (Crowe et al., Biochemistry, Epub Jul. 6, 2009). The ATPZ scaffold chemotype has not been identified in previous tau fibrillization screens and as these compounds showed drug-like structural features meriting their further characterization, we assessed whether this class of molecules interfered with the ability of tau to bind and stabilize MTs.

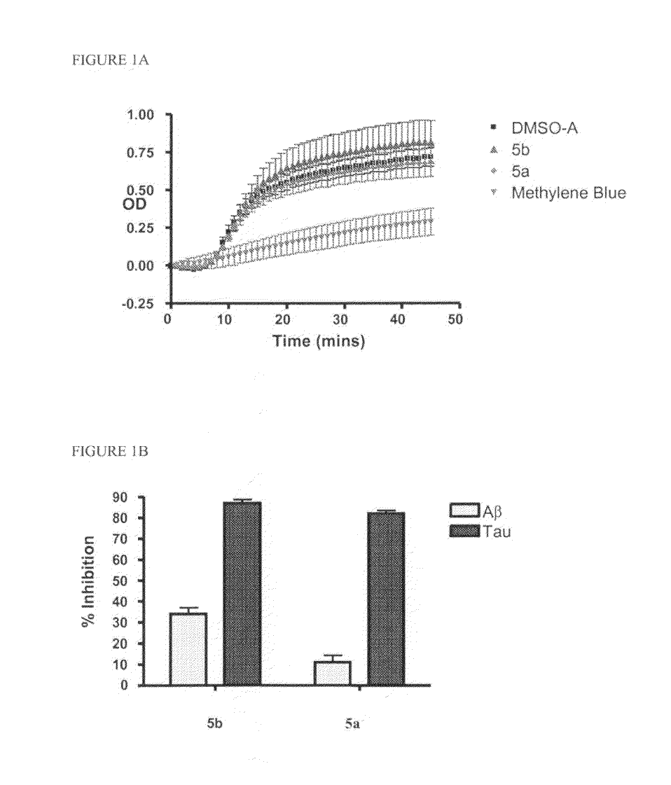

As depicted in FIG. 1A, neither of the ATPZ's had a meaningful effect on tau-mediated tubulin polymerization. In contrast, methylene blue, a promiscuous compound active in a high percentage of PubChem screens (data not shown), caused a significant diminution of tau-facilitated MT formation. The ATPZ's were also tested for their ability to block the fibrillization of Aβ(1-42), an amyloid peptide found within senile plaques in AD brain (Hardy, J. and Selkoe, D. J. (2002) Science 297, 353-356). The compounds were less effective in blocking Aβ(1-42) fibril formation, as judged by ThT fluorescence, than they were in inhibiting tau fibrillization (FIG. 1B). Thus, these data suggest that the ATPZ's preferentially block tau assembly, but the extent to which this selectivity extends to other amyloidogenic proteins awaits further testing.

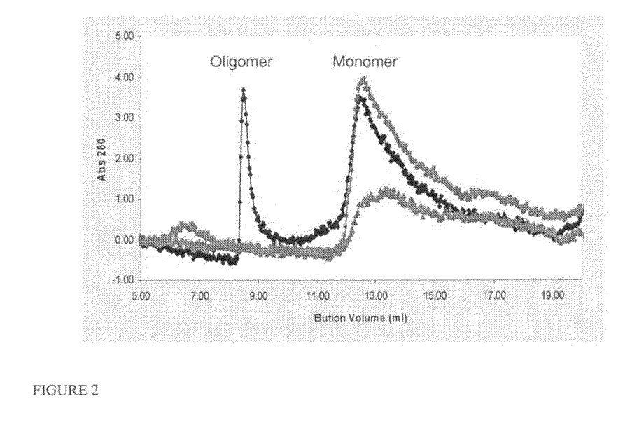

To further investigate the nature of the tau that remained in solution after incubation with an ATPZ inhibitor, aliquots of the post-centrifugation supernatants from full-length tau40 fibril assembly reactions conducted in the presence or absence of 50 μM 5b were analyzed by size-exclusion chromatography (SEC) to allow for the separation of monomeric tau40 from larger oligomeric species (Li, W. K. and Lee, V. M. Y. (2006) Biochemistry 45, 15692-15701). As shown in FIG. 2, inclusion of the ATPZ caused an appreciable increase in the amount of monomeric tau and also led to detectable oligomeric species within the post-centrifugation supernatant relative to the untreated fibrillization reaction. For comparison, the amount of monomeric tau in the supernatant fraction from a non-fibrillizing mixture of Tau40 and 5b incubated in the absence of heparin is also depicted in FIG. 2. These data suggest that 5b prevents the transition of tau oligomers to full fibrils, which could have beneficial consequences by preventing the formation of potentially neurotoxic fibrils and by increasing the pool of tau monomer that would be available to stabilize MTs.

The promising properties of the ATPZ class of tau assembly inhibitors led to the synthesis of additional analogs with the aim of evaluating possible SAR. As listed in Tables 3 and 4, a series of analogs were synthesized which exhibited a variety of substitutions of the ATPZ core structure. In addition, the previously tested compounds 5a and 5b were re-synthesized to confirm batch-to-batch reproducibility. All compound identities were confirmed by molecular mass determination, and were found to be least 85% pure. Identities of the synthesized analogs were further confirmed by NMR spectroscopic analysis The various ATPZ analogs were evaluated in the K18PL ThT and sedimentation assays to determine their IC₅₀ values and percent maximal inhibition (Tables 3-5). Interestingly, structural modifications in the fragment linked at C-4 appear to be generally well tolerated. Indeed, compounds bearing R3 modifications have been identified that appear to cause greater maximal inhibition in the ThT and centrifugation assays than the original hits identified during qHTS.

The ATPZ analogs were also examined in the Aβ(1-42) fibrillization assay to determine whether the relative lack of activity observed with the representatives from the screening library (FIG. 1B) was characteristic of the series as a whole. As summarized in Table 3, none of the ATPZ compounds caused >45% inhibition of Aβ(1-42) fibril formation when tested at 80 μM, and thus IC₅₀ values were not determined. Although there was some variation in the extent of maximal inhibition of Aβ(1-42) fibrillization within the series, it appears that in general the ATPZ's are significantly more effective at preventing the multimerization of tau than Aβ(1-42).

TABLE 3

| Comp. | 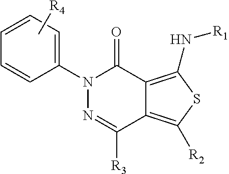 | | | | | Tau ThT Assay² Log % | | Tau Cent. Assay³ % | Aβ42 ThT Assay⁴ % |
|---|---|---|---|---|---|---|---|---|---|
| No. | R1 | R2 | R3 | R4 | PCP¹ | IC₅₀ | Inhib. | Soluble | Inhib. |
| 5b | H | H | —COOEt | 4-OMe | MW: 345.4 | −5.3 | 86 | 64 | 28 |
| | | | | | PSA: 94.2 | | | | |
| | | | | | ClogP: 2.6 | | | | |
| 9a | 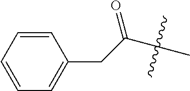 [SMILES: *C(=O)Cc1ccccc1] | H | —COOEt | 4-OMe | MW: 463.5 PSA: 97.3 ClogP: 4.3 | NC | 31 | 0 | ND |
| 9b | Ac— | H | —COOEt | H | MW: 357.4 | NC | 27 | 11 | ND |
| | | | | | PSA: 88 | | | | |
| | | | | | ClogP: 2.6 | | | | |
| 9c | 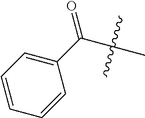 [SMILES: *C(=O)c1ccccc1] | H | —COOEt | H | MW: 419.5 PSA: 88.1 ClogP: 4.3 | NC | 8 | 2 | −7 |
| 16 | Ac | Me | H | H | MW: 299.3 | NC | 11 | 5 | −11 |
| | | | | | PSA: 61.8 | | | | |
| | | | | | ClogP: 2.9 | | | | |
| 5f | H | Me | —COOEt | H | MW: 329.4 | NC | 35 | 4 | 21 |
| | | | | | PSA: 85 | | | | |
| | | | | | ClogP: 2.8 | | | | |
| 8d | H | H | 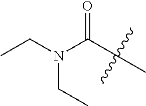 [SMILES: *C(=O)N(CC)CC] | 4-OMe | MW: 372.5 PSA: 88.2 ClogP: 2.5 | −5.0 | 95 | 99 | 35 |
| 15 | H | H | 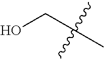 [SMILES: *CO] | H | MW: 273.3 PSA: 78.9 ClogP: 1.9 | −5.2 | 91 | 81 | 10 |
| 8c | H | H | 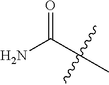 [SMILES: *C(N)=O] | H | MW: 286.3 PSA: 101.8 ClogP: 1.3 | −5.2 | 93 | 89 | 16 |
| 8a | H | H | 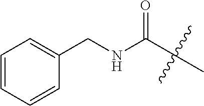 [SMILES: *C(=O)NCc1ccccc1] | H | MW: 376.4 PSA: 87.8 ClogP: 3.5 | −5.1 | 80 | 54 | 27 |
| 8b | H | H | 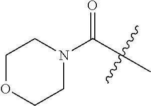 [SMILES: *C(=O)N1CCOCC1] | H | MW: 356.4 PSA: 88.2 ClogP: 1.4 | −4.8 | 81 | 89 | 0 |
| 14 | H | H | —NH₂ | H | MW: 258.3 | −5.2 | 92 | 86 | 21 |
| | | | | | PSA: 84.7 | | | | |
| | | | | | ClogP: 2.1 | | | | |
| 6a | H | H | —COOH | H | MW: 286.3 | −5.1 | 92 | 84 | 20 |
| | | | | | PSA: 98.8 | | | | |
| | | | | | ClogP: 2.2 | | | | |
| 6b | H | H | —COOH | 4-OMe | MW: 316.3 | −5.6 | 94 | 95 | 1 |
| | | | | | PSA: 108 | | | | |
| | | | | | ClogP: 2.1 | | | | |
| 5a | H | H | —COOEt | H | MW: 315.3 | −5.2 | 80 | 55 | −1 |
| | | | | | PSA: 85 | | | | |
| | | | | | ClogP: 2.7 | | | | |
| 17 | H | H | —COOEt | 4-Cl | MW: 349.8 | −5.4 | 84 | 62 | 41 |
| | | | | | PSA: 85 | | | | |
| | | | | | Clog: 3.3 | | | | |
| 5g | H | H | —COOEt | 3,4-Cl | MW: 384.2 | NC | 29 | 12 | 6 |
| | | | | | PSA: 85 | | | | |
| | | | | | ClogP: 3.9 | | | | |
| 5h | H | H | —COOEt | 4-Me | MW: 329.4 | −3.9 | 51 | 35 | 12 |
| | | | | | PSA: 85 | | | | |
| | | | | | ClogP: 3 | | | | |
| 5c | H | H | —COOEt | 3,4-Me | MW: 343.4 | NC | 48 | 28 | 7 |
| | | | | | PSA: 85 | | | | |
| | | | | | Clog: 3.3 | | | | |
| 18 | H | H | —COOMe | 4-F | MW: 319.3 | −5.3 | 86 | 64 | 29 |
| | | | | | PSA: 85 | | | | |
| | | | | | ClogP: 2.5 | | | | |
| 5d | H | H | —COOMe | 2-F | MW: 319.3 | NC | 28 | 20 | −5 |
| | | | | | PSA: 85 | | | | |
| | | | | | Clog: 2.5 | | | | |
| 19 | H | H | —COOMe | 3-CF₃ | MW: 369.3 | −5.0 | 66 | 41 | ND |
| | | | | | PSA: 85 | | | | |
| | | | | | ClogP: 3.3 | | | | |
| 5e | H | H | —COOEt | 4-COOEt | MW: 387.4 | NC | 29 | 23 | 4 |
| | | | | | PSA: 111.3 | | | | |
| | | | | | ClogP: 2.9 | | | | |

¹Calculated physical-chemical properties, including molecular weight (MW), polar surface area (PSA) and calculated log permeability (cLogP).
²IC₅₀ values for K18PL tau ThT fluorescence inhibition was as described in Experimental Procedures. The percent inhibition represents that obtained with the highest tested concentration (80 μM) of compound.
NC = not calculated due to insufficient activity.
³Secondary testing of compounds in the K18PL tau centrifugation assay as described in Experimental Procedures.
⁴Analysis of the compounds in the Aβ(1-42) fibrillization assay as described in Experimental Procedures. The percent inhibition represents that obtained with the highest tested concentration (80 μM) of compound.
ND = not determined

TABLE 4

| CNDR Comp. | 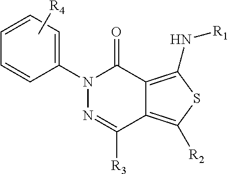 | | | | Tau ThT Assay² Log % | | Tau Cent. Assay³ % |
|---|---|---|---|---|---|---|---|
| No. | R1 | R2 | R3 | R4 | IC₅₀ | Inhib. | Soluble |
| 51395 | H | H | —COOH | 4-F | −5.7 | 84 | 83 |
| 51374 | H | H | —COOH | 4-OH | −5.4 | 84 | 70 |
| 51389 | H | H | —COOH | 4-Br | −5.5 | 95 | 78 |
| 51388 | H | H | —COOH | 3-Cl | −5.8 | 92 | 71 |
| 51370 | H | H | —COOH | 4-OCF₃ | −5.2 | 88 | 65 |
| 51405 | H | H | —Ph | 4-Cl | −6.0 | 85 | 44 |
| 51443 | H | H | —C(O)N(Et)₂ | 4-OCH₃ | −5.1 | 75 | 59 |
| 51397 | H | H | —C(O)NHc-Pr | 4-F | −5.6 | 88 | 78 |
| 51394 | H | H | —C(O)OEt | 4-F | −6.0 | 77 | 63 |
| 51355 | H | H | —C(O)OEt | 4-NH₂ | −5.9 | 74 | 67 |
| 51422 | H | H | —C(O)OEt | 3-Br | −6.0 | 87 | 67 |
| 51363 | H | H | —C(O)OEt | 4-OH | −5.3 | 81 | 69 |
| 51423 | H | H | —C(O)OEt | 3-CH₃ | −5.9 | 80 | 29 |
| 51404 | H | H | —C(O)OEt | 4-I | −5.6 | 89 | 63 |
| 51411 | H | —Br | —C(O)OEt | 4-Cl | −5.4 | 92 | 62 |
| 51439 | H | H | —C(O)OEt | 2-F | −6.1 | 48 | 41 |
| 51393 | H | —Cl | —C(O)OEt | 4-Cl | −5.7 | 88 | 65 |
| 51412 | H | H | —C(O)OEt | 4-N₃ | −5.2 | 80 | 51 |
| 51352 | H | H | —C(O)OEt | —NO₂ | −4.3 | 69 | 59 |
| 51373 | H | H | —C(O)OEt | 4-CF₃ | −5.0 | 48 | 27 |
| 51387 | H | H | —C(O)OEt | 3-Cl | −5.7 | 90 | 71 |
| 51358 | H | H | —C(O)OEt | 4-NHCH₃ | −5.4 | 76 | 61 |
| 51425 | H | H | —C(O)OEt | 2-Cl | −5.3 | 87 | 61 |
| 51369 | H | H | —C(O)OEt | 4-OCF₃ | −5.7 | 41 | 29 |
| 51359 | H | H | —C(O)OEt | 4-N(CH₃)₂ | −5.5 | 68 | 70 |
| 51421 | H | H | —C(O)OEt | 2-Br | −5.2 | 100 | 66 |
| 51424 | H | H | —C(O)OEt | 2-CH₃ | −5.9 | 82 | 58 |
| 51434 | H | H | —C(O)OEt | 2,4-dimethyl | −5.7 | 87 | 56 |
| 51420 | H | H | —C(O)OEt | 3-C(O)OEt | −5.7 | 74 | 83 |
| 51419 | H | H | —C(O)OEt | 2-C(O)OEt | −5.9 | 65 | 40 |
| 51347 | H | H | —C(O)OEt | 4-iPr | ND | 8 | 17 |
| 51435 | H | H | —C(O)OEt | 2-Ph | −6.1 | 78 | 65 |
| 51385 | H | H | —NHC(O)OEt | 4-Cl | −5.5 | 94 | 78 |
| 51391 | H | H | —C(O)NH—iPr | 3-Cl | −5.9 | 83 | 62 |
| 51396 | H | H | —C(O)NH—iPr | 4-F | −5.8 | 89 | 77 |
| 51376 | H | H | —C(O)NH—iPr | 4-OH | −4.8 | 86 | 84 |
| 51375 | H | H | —C(O)NH—iPr | 4-OCF₃ | −4.8 | 67 | 42 |
| 51400 | H | H | —CH₃ | 4-Cl | −5.9 | 87 | 90 |
| 51378 | H | H | —C(O)NHNH₂ | 4-Cl | −5.5 | 93 | 77 |
| 51433 | H | H | —C(O)NHCH₂CH₂OH | 4-Cl | −5.4 | 82 | 77 |
| 51383 | H | H | —NHC(O)OtBu | 4-Cl | −5.2 | 87 | 64 |
| 51360 | —CH₃ | H | —C(O)OEt | 4-Cl | −5.3 | 39 | 51 |

²IC₅₀ values for K18PL tau ThT fluorescence inhibition was as described in Experimental Procedures. The percent inhibition represents that obtained with the highest tested concentration (80 μM) of compound.
NC = not calculated due to insufficient activity.
³Secondary testing of compounds in the K18PL tau centrifugation assay as described in Experimental Procedures.
ND = not determined

### Compound Evaluation In Vitro.

The solubility of test compounds in the sodium acetate buffer used for subsequent fibrillization reactions was determined by turbidimetric measurements. Compounds were then evaluated in a heparin-induced tau assembly assay, in which the fibrillization of the truncated K18 tau fragment (comprised of four MT-binding repeats) bearing the P301L mutation (K18PL) found in FTDP-17, was monitored by thioflavine-T (ThT) binding and fluorescence (example in FIG. 3A). (Crowe, A.; et al., Identification of aminothienopyridazine inhibitors of tau assembly by quantitative high-throughput screening. Biochemistry 2009, 48, 7732-45.) The inhibitory activity of test compounds was then confirmed with an orthogonal sedimentation assay in which compound-treated fibrillizing mixtures were centrifuged and quantitation of K18PL tau in the soluble and insoluble fraction was performed by densitometric analyses of SDS-PAGE (FIG. 3B). As summarized in Tables 3-5, all active ATPZs exhibited IC₅₀ values in the 1-7 μM range in the primary ThT assay, with maximal percent inhibition of 70-90%. The activity against tau fibrillization was confirmed by sedimentation assay, where the majority of compounds exhibited 50% reduction in pelletable material compared to the untreated control. Since many of the ATPZ compounds were found to reach a solubility limit within the concentration range used in the biochemical assay, and considering that compound aggregation is known to be a potential mechanism of non specific inhibition (McGovern, S. L.; Helfand, B. T.; Feng, B.; Shoichet, B. K. A specific mechanism of nonspecific inhibition. J. Med. Chem. 2003, 46, 4265-72) additional experiments were conducted to ensure that the observed inhibitory activity of test compounds was not artifactual. Thus, the tau fibrillization assay was repeated in which both DMSO- and compound-treated mixtures were initially centrifuged for 30 min. prior to incubation (i.e., at time zero of the fibrillization reaction) to remove any pelletable material. As shown in FIG. 4A, no significant differences in compound-mediated inhibition of tau assembly were observed with and without the pre-centrifugation step. Furthermore, the IC₅₀ values of the centrifuged and control samples did not appear to differ appreciably (FIG. 4C). Moreover, SDS-PAGE analysis of the soluble and pelletable material after the initial centrifugation step revealed no significant difference between compound- and vehicle-treated mixtures; in all cases K18PL remained largely in the soluble fraction (FIG. 4B) and thus was not artifactually precipitated by the test compound. The activity and SDS-PAGE data suggest that the inhibition of K18PL fibrillization produced by the ATPZ analogs is not caused by insoluble or aggregated material. Furthermore, the absence of any correlation between compound solubility in buffer alone and activity in the tau fibrillization assay (Table 5) suggests that the solubilities of test compounds are somewhat increased in the presence of heparin and K18PL.

Notably, compounds E1, 17, 2, and F1-F7 also inhibited the aggregation of full-length tau (tau40) as determined by sedimentation assay, which revealed efficacy values in the 50-60% range (cf., Table 5). Compounds E1, 17, 2, and F1-F7 did not appear to interfere with the normal MT-stabilizing function of tau in a MT-polymerization assay (example shown in FIG. 5). Finally, to evaluate possible major toxicities associated with the ATPZs, all test compounds were evaluated in a cytotoxicity assay that employs rapidly dividing HEK-293 cells. All tested compounds were found to be non-toxic at 100 μM concentration.

### Pharmacokinetic Studies.

Test compounds underwent preliminary evaluations of brain penetration, in which 5 mg/Kg of each compound was administered intraperitoneally (i.p.) to a group of three normal mice; drug levels in brain and plasma were determined at a single time-point (1 h) by LC-MS/MS using pre-validated calibration curves. The results of these experiments, summarized in Table 5, revealed that with the exception of CNDR-51348 (E1), all other test compounds displayed significant brain uptake as demonstrated by the brain-to-plasma exposure ratios (B/P) above 0.3. The lack of brain penetration of CNDR-51348 was not unexpected given that the carboxylic acid moiety of this compound would be mostly negatively charged at physiological pH, and thus likely result in limited passive diffusion of the compound across the BBB. Conversely, the more lipophilic ester derivatives (CNDR-51346 and CNDR-51371) exhibited comparatively higher B/P ratio. In particular, CNDR-51371 (2) was found to reach significantly higher brain concentrations compared to the corresponding acid CNDR-51348. However, both esters appeared to have relatively short half-lives in plasma, as indicated by the limited amount of parent drug detected after 1 h from administration of the compounds. Moreover, monitoring for the hydrolyzed metabolite (i.e., CNDR-51348) in both brain and plasma revealed a considerable amount of acid CNDR-51348 in plasma, but not in the brain 1 h after administration of either ester. CNDR-51362 was selected for full PK analysis in which brain and plasma drug levels were determined at six time points (i.e., 30 min, 1 h, 2 h, 4 h, 8 h, and 16 h). The results from this experiment, illustrated in FIG. 6A, confirmed that the total B/P exposure ratio over 16 h is 1.6. Furthermore, these data reveal that CNDR-51362 exhibits good metabolic stability as indicated by an elimination half-life of ˜9.5 h in the brain and ˜7.4 h in plasma. Finally, in order to investigate the potential for ATPZ congeners to be orally administered, we evaluated brain and plasma level of CNDR-51362 and CNDR-51349 after oral administration of 5 mg/Kg. Notably, in both cases, significant concentrations of each test compound was achieved in the brain after oral administration (cf., FIGS. 6B and 6C). Comparison of the integrated area under the curve (AUC) in plasma after oral and i.v. administration of CNDR-51362 revealed an oral bioavailability of ˜64% (cf., FIGS. 6A and 6B).

Assuming that similar stoichiometric requirements also exist in vivo, any ATPZ candidate for in vivo efficacy study will have to reach free brain concentrations that are comparable to that of the unbound fraction of tau. The total intraneuronal tau concentration (i.e., MT-bound and MT-unbound tau) is estimated to be in the low μM range and, as previously noted, >99% of the protein is bound to MTs under physiological conditions. Although, the concentration of the MT-unbound fraction of tau is likely to increase in diseased neurons, this may be in the sub μM range. Thus, only compounds that readily cross the BBB would be viable candidates for in vivo evaluations of efficacy. Interestingly, preliminary evaluation of brain exposures of ATPZ test compounds revealed that with the exception of the acid derivative CNDR-51348, all other ATPZ congeners exhibited B/P ratios above 0.3. Considering that most CNS-active drugs typically exhibit B/P>0.3-0.5, these results indicate that ATPZs have the potential to achieve appreciable brain concentrations.

Furthermore, selected amide derivatives, such as CNDR-51349, CNDR-51362 and CNDR-51365, were found to reach brain concentrations above 800 ng/g (i.e., >2 μM) 1 h after i.p. administration of 5 mg/Kg. These results were further confirmed by complete PK studies on CNDR-51362, which displayed an average brain-to-plasma AUC exposure ratio of ˜1.6. Equally important, significant brain concentrations were also observed after oral administration of CNDR-51349 or CNDR-51362, with the oral bioavailability of the latter compound being ˜64%. Collectively, the results from the PK studies combined with the promising in vitro activity and safety data suggest that the ATPZ class of tau aggregation inhibitors hold considerable promise as candidate compounds for efficacy testing in transgenic mouse models of tauopathies.

TABLE 5

| 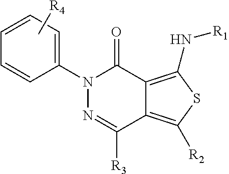 | | | | | Activity Against Tau Fibril Formation K18PL Tau40 | | | Brain and Plasma Levels at 1 h Time Point After I.P. Administration of 5 mg/kg of Test Compound | | |
|---|---|---|---|---|---|---|---|---|---|---|
| Cpd#/ | | | | Water | K18PL | Sed. | Sed. | Brain | Plasma | |
| CNDR# | R4 | R3 | PCP^{a} | Solubility | (ThT)^{b} | Assay^{c} | Assay^{d} | (ng/g) | (ng/ml) | B/P^{e} |
| CNDR-51348 | Cl | —COOH | MW: 321.7 | >200 μM | 1.6 μM^{ø} | 79% | 62% | 121 | 5226 | 0.023 |
| (E1) | | | PSA: 95.9 | | (87%)* | | | | | |
| | | | ClogP: 1.8* | | | | | | | |
| 17 | Cl | —COOEt | MW: 349.8 | 13 μM | 3.1 μM^{ø} | 70% | 48% | 67 | 20 | 3.3 |
| | | | PSA: 84.9 | | (71%)* | | | (8)^{†} | (3558) | |
| | | | CLogP: 2.6 | | | | | | | |
| CNDR-51371 | Cl | —COOiPr | MW: 363.8 | 32 μM | 1.5 μM^{ø} | 44% | 61% | 436 | 84 | 5.2 |
| (2) | | | PSA: 84.9 | | (81%)* | | | (0)^{†} | (1718)^{†} | |
| | | | ClogP: 2.9 | | | | | | | |
| CNDR-51367 | Cl | —CONHMe | MW: 334.8 | 13 μM | 4.3 μM^{ø} | 68% | 51% | 34 | 70 | 0.49 |
| (F1) | | | PSA: 87.8 | | (91%)* | | | | | |
| | | | ClogP: 0.9 | | | | | | | |
| CNDR-51365 | Cl | —CONHEt | MW: 348.8 | 32 μM | 5.6 μM^{ø} | 94% | 57% | 886 | 500 | 1.8 |
| (F2) | | | PSA: 87.8 | | (91%)* | | | | | |
| | | | ClogP: 1.4 | | | | | | | |
| CNDK-51349 | Cl | —CONHiPr | MW: 362.8 | 32 μM | 0.7 μM^{ø} | 65% | 45% | 1100 | 499 | 2.2 |
| (F3) | | | PSA: 87.8 | | (88%)* | | | | | |
| | | | ClogP: 1.8 | | | | | | | |
| CNDR-51362 | Cl | —CONH-cy-Pr | MW: 360.8 | 32 μM | 4.6 μM^{ø} | 69% | 58% | 1299 | 823 | 1.6 |
| (F4) | | | PSA: 87.8 | | (87%)* | | | | | |
| | | | ClogP: 1.5 | | | | | | | |
| CNDR-51361 | Cl | —CON(Me)₂ | MW: 348.8 | 32 μM | 5.3 μM^{ø} | 69% | 69% | 14 | 30 | 0.44 |
| (F5) | | | PSA: 79 | | (88%)* | | | | | |
| | | | ClogP: 0.6 | | | | | | | |
| CNDR-51366 | Cl | —CON(Me(Et) | MW: 362.8 | 80 μM | 6.8 μM^{ø} | 62% | 58% | 122 | 138 | 0.89 |
| (F6) | | | PSA: 79 | | (91%)* | | | | | |
| | | | ClogP: 1.1 | | | | | | | |
| CNDR-51390 | Br | —CONHiPr | MW: 407.3 | 13 μM | 2.2 μM^{ø} | 58% | 55% | 641 | 316 | 2.0 |
| (F7) | | | PSA: 87.8 | | (89%)* | | | | | |
| | | | ClogP: 1.9 | | | | | | | |

^{a}Physical-chemical properties (MW: molecular weight; PSA: polar surface area; ClogP: calculated partition coefficient between n-octanol and water);
^{b}Tau (K18PL) fibrillization assay monitored by thioflavine (ThT) binding and fluorescence;
^{c}Maximal percent inhibition of the K18PL fibrillization as determined by SDS-PAGE analyses of the soluble and insoluble fractions obtained after centrifugation of the fibrillizing K18PL mixtures;
^{d}Maximal percent inhibition of the tau40 fibrillization as determined by SDS-PAGE analyses of the soluble and insoluble fractions obtained after centrifugation of the fibrillizing tau40 mixtures;
^{e}Ratio between brain/plasma drug level, as determined by LC-MS/MS, 1 h after i.p. administration of 5 mg/Kg of test compound;
^{ø}IC₅₀ value;
*Maximal percent inhibition achieved in the assay;
^{†}Detected amount of hydrolysis product (i.e., CNDR-51348)

1. A method of treating an amyloid disease or disorder in a patient comprising administering to the patient a therapeutically effective amount of a compound of formula I: 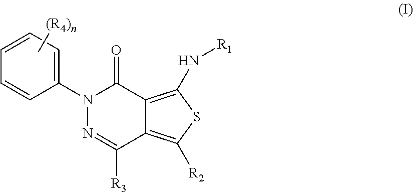
wherein:
R¹ is H, —C(O)—C₁₋₆alkyl; —C(O)—C₃₋₆cycloalkyl; —C(O)-aryl, —C(O)-heteroaryl; —C(O)alkaryl; —C(O)alkheteroaryl;
R² is H, halogen, C₁₋₆alkyl, or C₃₋₆cycloalkyl;
R³ is H, C₁₋₆alkyl; C₃₋₆cycloalkyl; C₁₋₆alkyl-OH; aryl; heteroaryl; cycloalkyl; heterocycloalkyl; —C(O)—C₁₋₆alkyl; —C(O)—C₃₋₆cycloalkyl; —C(O)—O—C₁₋₆alkyl; —C(O)—O—C₃₋₆cycloalkyl; —NHC(O)OC₁₋₆alkyl; —NHC(O)OC₃₋₆cycloalkyl; —COOH; —C(O)NR^{3a}R^{3b} wherein R^{3a} and R^{3b} are each independently H; —NH₂; C₁₋₆alkylene-OH; C₁₋₆alkyl; C₃₋₆cycloalkyl; aryl; heteroaryl; alkaryl; alkheteroaryl; or R^{3a} and R^{3b}, together with the nitrogen to which they are attached, form a 5-, 6-, or 7-membered heterocycloalkyl;
n is 0, 1, 2, 3, 4, or 5;
each R⁴ is independently C₁₋₆alkyl optionally substituted with 1-3 halogen; C₃₋₆cycloalkyl optionally substituted with 1-3 halogen; halogen; C₁₋₆alkoxy optionally substituted with 1-3 halogen; —OH, —N₃; —NO₂; —NR^{4a}R^{4b} wherein R^{4a} and R^{4b} are each independently H, C₁₋₆alkyl; C₃₋₆cycloalkyl; aryl; heteroaryl; alkaryl; alkheteroaryl; or R^{3a} and R^{3b}, together with the nitrogen to which they are attached, form a 5-, 6-, or 7-membered heterocycloalkyl; aryl; heterocycloalkyl; heteroaryl, —C(O)—C₁₋₆alkyl, —C(O)—C₃₋₆cycloalkyl —C(O)-aryl; —C(O)O—C₁₋₆alkyl; —C(O)O—C₃₋₆cycloalkyl; or —C(O)—Oaryl;
or a pharmaceutically acceptable salt form thereof.

2. The method of claim 1, wherein the amyloid disease or disorder is frontotemporal dementia, Alzheimer's disease, progressive supranuclear palsy, corticobasal degeneration, Parkinson's disease, or frontotemporal lobar degeneration.

3. The method of claim 1, wherein the tauopathy is Alzheimer's disease.

4. The method of claim 1, wherein:
R¹ is H, —C(O)—C₁₋₆alkyl; —C(O)—C₃₋₆cycloalkyl; —C(O)-aryl, —C(O)-heteroaryl; —C(O)alkaryl; —C(O)alkheteroaryl;
R² is H, C₁₋₆alkyl, or C₃₋₆cycloalkyl;
R³ is H, C₁₋₆alkyl; C₃₋₆cycloalkyl; C₁₋₆alkyl-OH; aryl; heteroaryl; cycloalkyl; heterocycloalkyl; —C(O)—C₁₋₆alkyl; —C(O)—C₃₋₆cycloalkyl; —C(O)—O—C₁₋₆alkyl; —C(O)—O—C₃₋₆cycloalkyl; —COOH; —C(O)NR^{3a}R^{3b} wherein R^{3a} and R^{3b} are each independently H; C₁₋₆alkyl; C₃₋₆cycloalkyl; aryl; heteroaryl; alkaryl; alkheteroaryl; or R^{3a} and R^{3b}, together with the nitrogen to which they are attached, form a 5-, 6-, or 7-membered heterocycloalkyl;
n is 0, 1, 2, 3, 4, or 5; and
each R⁴ is independently C₁₋₆alkyl optionally substituted with 1-3 halogen; C₃₋₆cycloalkyl optionally substituted with 1-3 halogen; halogen; C₁₋₆alkoxy; —OH; heterocycloalkyl; heteroaryl, —C(O)—C₁₋₆alkyl, —C(O)—C₃₋₆cycloalkyl —C(O)-aryl; —C(O)O—C₁₋₆alkyl; —C(O)O—C₃₋₆cycloalkyl; or —C(O)—Oaryl.

5. The method of claim 1, wherein
R¹ is H, —C(O)—C₁₋₆alkyl; —C(O)-aryl, —C(O)-heteroaryl; —C(O)alkaryl; —C(O)alkheteroaryl;
R² is H or C₁₋₆alkyl;
R³ is H, C₁₋₆alkyl; C₁₋₆alkyl-OH; aryl; heteroaryl; cycloalkyl; heterocycloalkyl; —C(O)—C₁₋₆alkyl; —COOH; —C(O)NR^{3a}R^{3b} wherein R^{3a} and R^{3b} are each independently H; C₁₋₆alkyl; aryl; heteroaryl; alkaryl; alkheteroaryl; or R^{3a} and R^{3b}, together with the nitrogen to which they are attached, form a 5-, 6-, or 7-membered heterocycloalkyl; and
each R⁴ is independently C₁₋₆alkyl optionally substituted with 1-3 halogen; halogen; C₁₋₆alkoxy; —OH, heterocycloalkyl; heteroaryl, —C(O)—C₁₋₆alkyl, —C(O)-aryl; —C(O)O—C₁₋₆alkyl; or —C(O)—Oaryl.

6. The method of claim 1, wherein R¹ is H.

7. The method of claim 1, wherein R² is H.

8. The method of claim 1, wherein R² is halogen.

9. The method of claim 1, wherein R³ is —C(O)—O—C₁₋₆alkyl.

10. The method of claim 1, wherein R³ is —C(O)—O—CH₃ or —C(O)—O—CH₂CH₃.

11. The method of claim 1, wherein R³ is —COOH.

12. The method of claim 1, wherein R³ is —C(O)NR^{3a}R^{3b}.

13. The method of claim 1, wherein R³ is C₁₋₆alkylene-OH.

14. The method of claim 1, wherein R³ is —NHC(O)OC₁₋₆alkyl or —NHC(O)OC₃₋₆cycloalkyl.

15. The method of claim 1, wherein R³ is —NH₂.

16. The method of claim 1, wherein R^{3a} and R^{3b} are each H.

17. The method of claim 1, wherein R^{3a} and R^{3b} are each C₁₋₆alkyl.

18. The method of claim 1, wherein R^{3a} is C₁₋₆alkyl and R^{3b} is H.

19. The method of claim 1, wherein R^{3a} is C₁₋₆cycloalkyl and R^{3b} is H.

20. The method of claim 1, wherein R^{3a} is alkaryl and R^{3b} is H.

21. The method of claim 1, wherein R^{3a} and R^{3b}, together with the nitrogen to which they are attached, form a 5-, 6-, or 7-membered heterocycloalkyl.

22. The method of claim 21, wherein the heterocycloalkyl is piperidyl.

23. The method of claim 21, wherein the heterocycloalkyl is morpholinyl.

24. The method of claim 1, wherein the compound of formula I is selected from the following table:

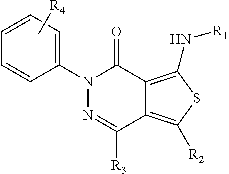

| Comp No. | R1 | R2 | R3 | R4 |
|---|---|---|---|---|
| 5b | H | H | —COOEt | 4-OMe |
| 9a | 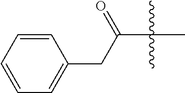 [SMILES: *C(=O)Cc1ccccc1] | H | —COOEt | 4-OMe |
| 9b | Ac— | H | —COOEt | H |
| 9c | 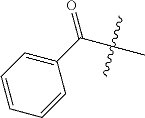 [SMILES: *C(=O)c1ccccc1] | H | —COOEt | H |
| 16 | Ac | Me | H | H |
| 5f | H | Me | —COOEt | H |
| 8d | H | H | 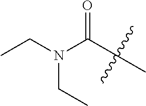 [SMILES: *C(=O)N(CC)CC] | H |
| 15 | H | H | 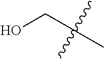 [SMILES: *CO] | H |
| 8c | H | H | 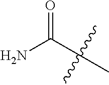 [SMILES: *C(N)=O] | H |
| 8a | H | H | 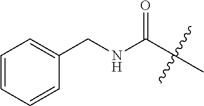 [SMILES: *C(=O)NCc1ccccc1] | H |
| 8b | H | H | 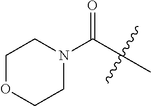 [SMILES: *C(=O)N1CCOCC1] | H |
| 14 | H | H | —NH₂ | H |
| 6a | H | H | —COOH | H |
| 6b | H | H | —COOH | 4-OMe |
| 5a | H | H | —COOEt | H |
| 17 | H | H | —COOEt | 4-Cl |
| 5g | H | H | —COOEt | 3,4-dichloro |
| 5h | H | H | —COOEt | 4-Me |
| 5c | H | H | —COOEt | 3,4-dimethyl |
| 18 | H | H | —COOMe | 4-F |
| 5d | H | H | —COOEt | 2-F |
| 19 | H | H | —COOMe | 3-CF₃ |
| 5e | H | H | —COOEt | 4-COOEt |
| CNDR-51371 | H | H | —COOiPr | 4-Cl |
| (2) | | | | |
| CNDR-51367 | H | H | —CONHMe | 4-Cl |
| (F1) | | | | |
| CNDR-51365 | H | H | —CONHEt | 4-Cl |
| (F2) | | | | |
| CNDR-51349 | H | H | —CONHiPr | 4-Cl |
| (F3) | | | | |
| CNDR-51362 | H | H | —CONH—c-Pr | 4-Cl |
| (F4) | | | | |
| CNDR-51361 | H | H | —CON(Me)₂ | 4-Cl |
| (F5) | | | | |
| CNDR-51366 | H | H | —CON(Me)Et | 4-Cl |
| (F6) | | | | |
| CNDR-51390 | H | H | —CONHiPr | 4-Br |
| (F7) | | | | |
| CNDR-51386 | H | H | —COOEt | 4-Br |
| (D2) | | | | |
| CNDR-51348 | H | H | —COOH | 4-Cl |
| (El) | | | | |
| CNDR-51389 | H | H | —COOH | 4-Br |
| (E2) | | | | |
| CNDR-51443 | H | H | —C(O)N(Et)₂ | 4-OCH₃ |
| (F10) | | | | |
| CNDR-51394 | H | H | —COOEt | 4-F |
| (D3) | | | | |
| CNDR-51363 | H | H | —COOEt | 4-OH |
| (D11) | | | | |
| CNDR-51423 | H | H | —COOEt | 3-CH₃ |
| (D15) | | | | |
| CNDR-51404 | H | H | —COOEt | 4-I |
| (D4) | | | | |
| CNDR-51439 | H | H | —COOEt | 2-F |
| CNDR-51352 | H | H | —COOEt | 4-NO₂ |
| (D5) | | | | |
| CNDR-51373 | H | H | —COOEt | 4-CF₃ |
| (D12) | | | | |
| CNDR-51387 | H | H | —COOEt | 3-Cl |
| CNDR-51425 | H | H | —COOEt | 2-Cl |
| (D19) | | | | |
| CNDR-51369 | H | H | —COOEt | 4-OCF₃ |
| (D10) | | | | |
| CNDR-51424 | H | H | —COOEt | 2-CH₃ |
| (D23) | | | | |
| CNDR-51347 | H | H | —COOEt | 4-iPr |
| (D13) | | | | |

25. The method of claim 1, wherein the compound of formula I is selected from the following table

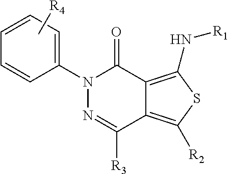

| Comp No. | R1 | R2 | R3 | R4 |
|---|---|---|---|---|
| 5f | H | Me | —COOEt | H |
| 8d | H | H | 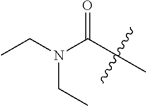 [SMILES: *C(=O)N(CC)CC] | 4-OCH₃ |
| 8c | H | H | 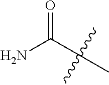 [SMILES: *C(N)=O] | H |
| 8a | H | H | 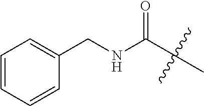 [SMILES: *C(=O)NCc1ccccc1] | H |
| 8b | H | H | 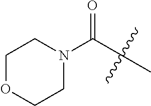 [SMILES: *C(=O)N1CCOCC1] | H |
| 14 | H | H | —NH₂ | H |
| 5g | H | H | —COOEt | 3,4-dichloro |
| 18 | H | H | —COOMe | 4-F |
| 5d | H | H | —COOEt | 2-F |
| 19 | H | H | —COOMe | 3-CF₃ |
| 5e | H | H | —COOEt | 4-COOEt |
| CNDR-51367 | H | H | —CONHMe | 4-Cl |
| (F1) | | | | |
| CNDR-51365 | H | H | —CONHEt | 4-Cl |
| (F2) | | | | |
| CNDR-51349 | H | H | —CONHiPr | 4-Cl |
| (F3) | | | | |
| CNDR-51362 | H | H | —CONH—c-Pr | 4-Cl |
| (F4) | | | | |
| CNDR-51361 | H | H | —CON(Me)₂ | 4-Cl |
| (F5) | | | | |
| CNDR-51366 | H | H | —CON(Me)Et | 4-Cl |
| (F6) | | | | |
| CNDR-51390 | H | H | —CONHiPr | 4-Br |
| (F7) | | | | |
| CNDR-51386 | H | H | —COOEt | 4-Br |
| (D2) | | | | |
| CNDR-51348 | H | H | —COOH | 4-Cl |
| (E1) | | | | |
| CNDR-51389 | H | H | —COOH | 4-Br |
| (E2) | | | | |
| CNDR-51395 | H | H | —COOH | 4-F |
| (E3) | | | | |
| CNDR-51374 | H | H | —COOH | 4-OH |
| (E4) | | | | |
| CNDR-51388 | H | H | —COOH | 3-Cl |
| (E6) | | | | |
| CNDR-51370 | H | H | —COOH | 4-OCF₃ |
| (E5) | | | | |
| CNDR-51405 | H | H | —Ph | 4-Cl |
| CNDR-51397 | H | H | —C(O)NH—cPr | 4-F |
| (F9) | | | | |
| CNDR-51355 | H | H | —C(O)OEt | 4-NH₂ |
| (D7) | | | | |
| CNDR-51422 | H | H | —C(O)OEt | 3-Br |
| (D14) | | | | |
| CNDR-51411 | H | Br | —C(O)OEt | 4-Cl |
| CNDR-51393 | H | Cl | —C(O)OEt | 4-Cl |
| CNDR-51412 | H | H | —C(O)OEt | 4-N₃ |
| CNDR-51358 | H | H | —C(O)OEt | 4-NHCH₃ |
| (D8) | | | | |
| CNDR-51359 | H | H | —C(O)OEt | 4-N(CH₃)₂ |
| (D9) | | | | |
| CNDR-51421 | H | H | —C(O)OEt | 2-Br |
| (D20) | | | | |
| CNDR-51434 | H | H | —C(O)OEt | 2,4, |
| (D24) | | | | dimethyl |
| CNDR-51420 | H | H | —C(O)OEt | 3-C(O)OEt |
| (D17) | | | | |
| CNDR-51419 | H | H | —C(O)OEt | 2-C(O)OEt |
| (D21) | | | | |
| CNDR-51435 | H | H | —C(O)OEt | 2-Ph |
| (D22) | | | | |
| CNDR-51385 | H | H | —NHC(O)OEt | 4-Cl |
| CNDR-51391 | H | H | —C(O)NH—iPr | 3-Cl |
| CNDR-51396 | H | H | —C(O)NH—iPr | 4-F |
| (F8) | | | | |
| CNDR-51376 | H | H | —C(O)NH—iPr | 4-OH |
| (F13) | | | | |
| CNDR-51375 | H | H | —C(O)NH—iPr | 4-OCF₃ |
| (F12) | | | | |
| CNDR-51400 | H | H | —CH₃ | 4-Cl |
| CNDR-51378 | H | H | —C(O)NHNH₂ | 4-Cl |
| CNDR-51433 | H | H | —C(O)NHCH₂CH₂OH | 4-Cl |
| (F14) | | | | |
| CNDR-51383 | H | H | —NHC(O)OtBu | 4-Cl |
| CNDR-51360 | CH₃ | H | —C(O)OEt | 4-Cl |

26. A compound selected from the following table:

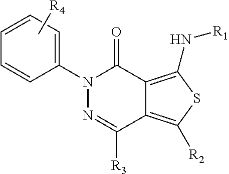

| Comp No. | R1 | R2 | R3 | R4 |
|---|---|---|---|---|
| 5f | H | Me | —COOEt | H |
| 8d | H | H | 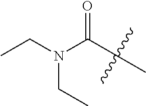 [SMILES: *C(=O)N(CC)CC] | 4-OCH₃ |
| 8c | H | H | 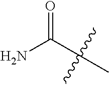 [SMILES: *C(N)=O] | H |
| 8a | H | H | 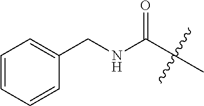 [SMILES: *C(=O)NCc1ccccc1] | H |
| 8b | H | H | 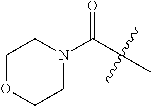 [SMILES: *C(=O)N1CCOCC1] | H |
| 14 | H | H | —NH₂ | H |
| 5g | H | H | —COOEt | 3,4-dichloro |
| 18 | H | H | —COOMe | 4-F |
| 5d | H | H | —COOEt | 2-F |
| 19 | H | H | —COOMe | 3-CF₃ |
| 5e | H | H | —COOEt | 4-COOEt |
| CNDR-51367 | H | H | —CONHMe | 4-Cl |
| (F1) | | | | |
| CNDR-51365 | H | H | —CONHEt | 4-Cl |
| (F2) | | | | |
| CNDR-51349 | H | H | —CONHiPr | 4-Cl |
| (F3) | | | | |
| CNDR-51362 | H | H | —CONH—c-Pr | 4-Cl |
| (F4) | | | | |
| CNDR-51361 | H | H | —CON(Me)₂ | 4-Cl |
| (F5) | | | | |
| CNDR-51366 | H | H | —CON(Me)Et | 4-Cl |
| (F6) | | | | |
| CNDR-51390 | H | H | —CONHiPr | 4-Br |
| (F7) | | | | |
| CNDR-51386 | H | H | —COOEt | 4-Br |
| (D2) | | | | |
| CNDR-51348 | H | H | —COOH | 4-Cl |
| (E1) | | | | |
| CNDR-51389 | H | H | —COOH | 4-Br |
| (E2) | | | | |
| CNDR-51395 | H | H | —COOH | 4-F |
| (E3) | | | | |
| CNDR-51374 | H | H | —COOH | 4-OH |
| (E4) | | | | |
| CNDR-51388 | H | H | —COOH | 3-Cl |
| (E6) | | | | |
| CNDR-51370 | H | H | —COOH | 4-OCF₃ |
| (E5) | | | | |
| CNDR-51405 | H | H | —Ph | 4-Cl |
| CNDR-51397 | H | H | —C(O)NH—cPr | 4-F |
| (F9) | | | | |
| CNDR-51355 | H | H | —C(O)OEt | 4-NH₂ |
| (D7) | | | | |
| CNDR-51422 | H | H | —C(O)OEt | 3-Br |
| (D14) | | | | |
| CNDR-51411 | H | Br | —C(O)OEt | 4-Cl |
| CNDR-51393 | H | Cl | —C(O)OEt | 4-Cl |
| CNDR-51412 | H | H | —C(O)OEt | 4-N₃ |
| CNDR-51358 | H | H | —C(O)OEt | 4-NHCH₃ |
| (D8) | | | | |
| CNDR-51359 | H | H | —C(O)OEt | 4-N(CH₃)₂ |
| (D9) | | | | |
| CNDR-51421 | H | H | —C(O)OEt | 2-Br |
| (D20) | | | | |
| CNDR-51434 | H | H | —C(O)OEt | 2,4, |
| (D24) | | | | dimethyl |
| CNDR-51420 | H | H | —C(O)OEt | 3-C(O)OEt |
| (D17) | | | | |
| CNDR-51419 | H | H | —C(O)OEt | 2-C(O)OEt |
| (D21) | | | | |
| CNDR-51435 | H | H | —C(O)OEt | 2-Ph |
| (D22) | | | | |
| CNDR-51385 | H | H | —NHC(O)OEt | 4-Cl |
| CNDR-51391 | H | H | —C(O)NH—iPr | 3-Cl |
| CNDR-51396 | H | H | —C(O)NH—iPr | 4-F |
| (F8) | | | | |
| CNDR-51376 | H | H | —C(O)NH—iPr | 4-OH |
| (F13) | | | | |
| CNDR-51375 | H | H | —C(O)NH—iPr | 4-OCF₃ |
| (F12) | | | | |
| CNDR-51400 | H | H | —CH₃ | 4-Cl |
| CNDR-51378 | H | H | —C(O)NHNH₂ | 4-Cl |
| CNDR-51433 | H | H | —C(O)NHCH₂CH₂OH | 4-Cl |
| (F14) | | | | |
| CNDR-51383 | H | H | —NHC(O)OtBu | 4-Cl |
| CNDR-51360 | CH₃ | H | —C(O)OEt | 4-Cl |
